(19) United States
(12) Patent Application Publication
(10) Pub. No.: US 2022/0249597 A1
(43) Pub. Date: Aug. 11, 2022

(54) MIF INHIBITORS AND METHODS OF USE THEREOF

(71) Applicant: The Johns Hopkins University, Baltimore, MD (US)

(72) Inventors: Ted M. Dawson, Baltimore, MD (US); Valina L. Dawson, Baltimore, MD (US); Yingfei Wang, Baltimore, MD (US); Hyejin Park, Baltimore, MD (US); Jun Liu, Baltimore, MD (US); Hanjing Peng, Baltimore, MD (US); Tae-In Kam, Baltimore, MD (US)

(21) Appl. No.: 17/675,875

(22) Filed: Feb. 18, 2022

Related U.S. Application Data
(63) Continuation of application No. 16/330,061, filed on Mar. 1, 2019, now abandoned.
(60) Provisional application No. 62/383,209, filed on Sep. 2, 2016.

(51) Int. Cl. A61K 38/12; C12Q 1/6834; A61P 25/28; G01N 33/50; A61P 25/00

(52) U.S. Cl. CPC A61K 38/12; C12Q 1/6834; A61P 25/00; G01N 33/5008; A61P 25/28

(57) ABSTRACT

Provided herein are methods of treating a disease, such as Parkinson's disease, that is due to increased poly [ADP-ribose] polymerase 1 (PARP-1) activation, by inhibiting macrophage migration inhibitory factor (MIF) nuclease activity.

### RELATED APPLICATIONS

This application is a Continuation of U.S. patent application Ser. No. 16/330,061 filed Mar. 1, 2019, pending, which is a 371 National Stage Application of International Application No. PCT/US17/49778, filed Aug. 31, 2017, which claims the benefit under 35 U.S.C. § 119(e) to U.S. Provisional Application No. 62/383,209, filed on Sep. 2, 2016, the entire contents of which is incorporated herein by reference in its entirety.

### GRANT INFORMATION

This invention was made with government support under NS078049, DA000266, NS067525, and NS038377 awarded by the National Institutes of Health. The government has certain rights in the invention.

### INCORPORATION OF SEQUENCE LISTING

The material in the accompanying sequence listing is hereby incorporated by reference into this application. The accompanying sequence listing text file, name JHU4070_2_ST25.txt, was created on Feb. 16, 2022, and is 40.8 kb. The file can be accessed using Microsoft Word on a computer that uses Windows OS.

### BACKGROUND OF THE INVENTION

### Field of the Invention

The invention relates generally to macrophage migration inhibitory factor (MIF) and more specifically to the use of MIF inhibitors in the treatment of diseases.

### Background Information

Poly(ADP-ribose) (PAR) polymerase-1 (PARP-1) is an important nuclear enzyme that is activated by DNA damage where it facilitates DNA repair (1). Excessive activation of PARP-1 causes an intrinsic caspase-independent cell death program designated parthanatos (2, 3), which plays a prominent role following a number of toxic insults in many organ systems (4, 5), including ischemia-reperfusion injury after stroke and myocardial infarction, inflammatory injury, reactive oxygen species-induced injury, glutamate excitotoxicity and neurodegenerative diseases such as Parkinson disease and Alzheimer disease (2, 4, 6). Consistent with the idea that PARP-1 is a key cell death mediator, PARP inhibitors or genetic deletion of PARP-1 are profoundly protective against these and other cellular injury paradigms and models of human disease (2, 4, 5, 7).

Molecular mechanisms underlying parthanatos involve PAR-dependent apoptosis-inducing factor (AIF) release from the mitochondria and translocation to the nucleus resulting in fragmentation of DNA into 20-50 kb fragments (2, 8-11). AIF itself has no obvious nuclease activity (2). Although it has been suggested that CED-3 Protease Suppressor (CPS)-6, an endonuclease G (EndoG) homolog in Caenorhabditis elegans (C. elegans), cooperates with the worm AIF Homolog (WAH-1) to promote DNA degradation (12), EndoG does not seem to play an essential role in PARP-dependent chromatinolysis and cell death after transient focal cerebral ischemia in mammals (13). The nuclease responsible for the chromatinolysis during parthanatos is not known.

### SUMMARY OF THE INVENTION

The present invention is based on the identification of macrophage migration inhibitory factor (MIF) as a PARP-1 dependent AIF-associated nuclease (PAAN).

In one embodiment, the invention provides a method of treating a disease associated with increased poly [ADP-ribose] polymerase 1 (PARP-1) activation in a subject. The method includes administering to the subject a therapeutically effective amount of an inhibitor of macrophage migration inhibitory factor (MIF) nuclease activity, thereby treating or alleviating the symptoms of the disease.

In one aspect, the disease is an inflammatory disease. In another aspect, the inflammatory disease is Alzheimer's, ankylosing spondylitis, arthritis, osteoarthritis, rheumatoid arthritis, psoriatic arthritis, asthma atherosclerosis, Crohn's disease, colitis, dermatitis diverticulitis, fibromyalgia, hepatitis, irritable bowel syndrome, systemic lupus erythematous, nephritis, ulcerative colitis or Parkinson's disease.

In one embodiment, the inhibitor is a macrocyclic rapafucin compound, e.g., from a hybrid macrocyclic rapafucin library.

The invention also provides a method of screening for macrophage migration inhibitory factor (MIF) inhibitors, including steps such as immobilizing a single-stranded amine modified MIF target DNA, followed by incubating MIF with and without a compound from a macrocyclic rapafucin library; the single-stranded amine modified MIF target DNA is hybridized with biotinylated DNA that is complementary to the single-stranded amine modified MIF target DNA, followed by incubating with streptavidin enzyme conjugate followed by a substrate, wherein the substrate is acted upon by the streptavidin enzyme conjugate. The absorbance of MIF with a library compound is compared to the absorbance of MIF without a library compound in order to determine whether a compound is an inhibitor or not based upon changes in absorbance.

### BRIEF DESCRIPTION OF THE DRAWINGS

FIGS. 1A-1E. FIG. 1. Identification of MIF as the key cell death effector mediating PARP-1 dependent cell death. (A) Strategy for identifying AIF-associated proteins involved in PARP-1 dependent cell death. (B) siRNA-based PARP-1-dependent cell viability high-throughput screening in HeLa cells 24 h after MNNG treatment (50 μM, 15 min). n=8. The experiments were repeated in 4 independent tests. (C) Schematic representation of MIF's PD-D/E(X)K domains. (D) Alignment of the nuclease domain of human MIF and other nucleases. Arrows above the sequence indicate β-strands and rectangles represent α-helices. Amino acid residues that were mutated are indicated with an arrow and number (see Results). Nuclease and CxxCxxHx₍ₙ₎C domains are highlighted in green and pink respectively. (E) Crystal structure of MIF trimer (pdb:1GD0) (left) and MIF PD-D/E(x)K motif in trimer (right).

FIGS. 2A-2D. FIG. 2. MIF is a novel nuclease that cleaves genomic DNA. (A) In vitro MIF nuclease assay using pcDNA as substrate. (B) In vitro pulse-field gel electrophoresis-MIF nuclease assay using human genomic DNA as a substrate in buffer containing Mg²⁺ (10 mM) with or without EDTA (50 mM) or Ca²⁺ (2 mM) with or without EDTA (25 mM). (C) Pulse-field gel electrophoresis assay of MNNG-induced DNA damage in MIF deficient HeLa cells and wild type HeLa cells treated with or without DPQ (30 μM) or ISO-1 (100 μM). (D) Nuclease assay of MIF WT and mutants using human genomic DNA as the substrate.

FIGS. 3A-3E. FIG. 3. MIF binds and cleaves single stranded DNA. (A) MIF DNA binding motif determined by ChIP-seq. (B) MIF binds to ssDNA, but not double strand DNA, with the structure specificity. 5′ biotin-labeled small DNA substrates with different structures or different sequences were used in the EMSA assay (see FIG. 19) for illustrations of substrates and Table 1 for sequences). (C) MIF cleaves unpaired bases at the 3′ end of stem loop ssDNA with the structure specificity. 5′ or 3′ biotin-labeled small DNA substrates with different structures or different sequences were used in the nuclease assay (see FIG. 19) for illustrations of substrates and Table 1 for sequences). Experiments were replicated for 4 times using MIF protein purified from 3 independent preparations. (D) MIF cleaves 3′ unpaired bases from non-labeled PS³⁰ and 3F1 substrates. Ladder 1 and 2 were customized using PS³⁰ and its cleavage products by removing its 3′ nucleotide one by one. Ladder 1 was prepared using PS³⁰, PS²⁸, PS²⁶, PS²⁴, PS²² and PS²⁰. Ladder 2 was prepared using PS²⁹, PS²⁷, PS²⁵, PS²³ and PS²¹. (E) MIF cleavage sites on non-labeled PS³⁰ and 3F1 substrates.

FIGS. 4A-4L. FIG. 4. AIF is required for the recruitment of MIF to the nucleus after NMDA treatment. (A) GST-pulldown assay of immobilized GST-MIF WT and GST-MIF variant binding to AIF. (B) Nuclease activity and AIF-binding activity of MIF WT and MIF variants. (C-D) Co-immunoprecipitation of MIF and AIF in cortical neurons under physiological and NMDA treated conditions. Star indicates IgG. (E-G) Nuclear translocation of AIF and MIF after NMDA treatment in wild type, AIF knockdown and MIF knockdown cortical neurons. Intensity of AIF and MIF signal in postnuclear fraction (PN) and nuclear fraction (N) is shown in G. (H) Expression of MIF in WT and KO neurons. (I) Co-immunoprecipitation of Flag-tagged MIF variants and AIF in cortical neurons after NMDA treatment. (J-L), Nuclear translocation of AIF and exogenous MIF WT and MIF variants after NMDA treatment in MIF KO cortical neurons. Scale bar, 20 μm. Intensity of AIF and MIF signal in postnuclear fraction (PN) and nuclear fraction (N) is shown in L. Means±SEM are shown. Experiments were replicated at least 3 times. *P<0.05, **P<0.01, ***P<0.001, Student's t test (D) and one-way ANOVA (G, L).

FIGS. 5A-5G. FIG. 5. MIF nuclease activity is critical for DNA damage and ischemic neuronal cell death in vitro and in vivo. (A) NMDA (500 μM, 5 min)-induced cytotoxicity in MIF WT, KO and KO cortical neurons expressing MIF WT, E22Q or E22A. (B) Representative images of NMDA-caused DNA damage 6 h after the treatment determined by comet assay in MIF WT, KO and KO neurons expressing MIF WT, E22Q or E22A. Dashed lines indicate the center of the head and tail. Scale bar, 20 μm. (C) Pulse-field gel electrophoresis assay of NMDA-induced DNA damage 6 h after treatment in MIF WT and KO neurons and KO neurons expressing MIF WT, E22Q and E22A. (D) Representative images of TTC staining of MIF WT, KO and KO mice which were injected with AAV2-MIF WT, E22Q or E22A 24 h after 45 min MCAO. (E) Quantification of infarction volume in cortex, striatum and hemisphere 1 day or 7 days after 45 min MCAO. (F-G) Neurological deficit was evaluated by open field on a scale of 0-5 at 1 day, 3 days or 7 days after MCAO surgery. WT MCAO (n=29), KO MCAO (n=20), KO-WT MCAO (n=23). KO-E22Q MCAO (n=22), KO-E22A MCAO (n=19). Means±SEM are shown in A, E, F, G. *P<0.05 (E,F), ***P<0.001 (A, E), one-way ANOVA. ***P<0.001 (G), WT versus KO, KO-WT versus KO-E22Q/KO-E22A at different time points, two-way ANOVA.

FIG. 6. Establishment of MIF inhibitor screening using macrocyclic compound library. The schematic representation of macrocyclic screening for MIF inhibitors based on cleavage assay. Single-strand amine-modified oligonucleotides (MIF target DNA) were immobilized on DNA-BIND plates and incubated in MIF protein with or without inhibitors. After MIF cleavage, the fragments were hybridized with biotin-labeled complementary oligonucleotides and detected by monitoring absorbance at 450 nm.

FIG. 7. Schematic representation of macrocyclic rapafucin libraries.

FIG. 8. The result of screening for MIF inhibitors. Scatter plot of percentage inhibition of MIF cleavage from 38 plates of the macrocyclic library. The blue line is the positive control incubated without MIF and green line is the negative control incubated with MIF. Right graph represents the histogram of the compounds tested.

FIG. 9. The results of individual compounds screening for MIF inhibitors. Scatter plot of the percentage inhibition of MIF cleavage (X axis) and the inhibition of MNNG-induced cell death (Y axis).

FIGS. 10A-10B. FIG. 10. Dose-dependent confirmation of 4 hits. (A) 4 candidates were assessed for cytoprotection in HeLa cells treated with MNNG. The candidates provide dose-dependent cytoprotection. (B) 4 candidates were subjected to cleavage assay in TBE gel. The candidates can prevent the cleavage of substrates by MIF.

FIG. 11. Primary cortical neurons were treated with PFF with or without 2 hits for 14 days. Images show the PFF-induced cell death by 2 hits (left). Scale bar, 50 μm. Quantification of PFF-induced cell death by 2 hits. Bars reflect the means±s.d. from three experiments. **P<0.005, ***P<0.001 (two-tailed unpaired t-test).

FIGS. 12A-12C. FIG. 12. EndoG is not required for PARP-1 dependent cell death. (A) Knockout endoG using CRISPR-Cas9 system in SH-SY5Y cells. EV, empty vector. (B) Knockout endoG has no effect on MNNG-induced cell death. (C) Knockout endoG has no effect on MNNG caused DNA damage.

FIGS. 13A-13H. FIG. 13. MIF knock down protects cells from MNNG and NMDA-induced cell death. (A) Representative images of HeLa cells transduced with human MIF shRNA1-3 IRES-GFP lentivirus or non-targeting (NT) shRNA IRES-GFP lentivirus. (B) MIF protein levels in HeLa cells after shRNA transduction. hMIF shRNA 1, 2 and 3 caused 83.3±7.1%, 71.6±3.2%, and 82.7±6.3% MIF protein reduction in HeLa cells. (C) Quantification of MNNG (50 μM, 15 min)-induced HeLa cell death. Means±SEM are shown. ***P<0.001, versus DMSO control. ###P<0.001, versus WT with MNNG treatment. (D) Representative images of cortical neurons transduced with mouse MIF shRNA1-3 IRES-GFP or non-targeting (NT) shRNA IRES-GFP lentivirus. (E) MIF protein levels in cortical neurons after shRNA transduction. (F) Quantification of NMDA (500 μM, 5 min)-induced neuronal cell death in MIF knockdown neurons. mMIF shRNA 1, 2 and 3 caused 84.5±8.2%, 90.1±7.1%, and 92.2±3.3% MIF protein reduction in cortical neuron. Means±SEM are shown. ***P<0.001, versus CSS control. ###P<0.001, versus WT with NMDA treatment. (G) Representative immunoblots of MIF knockdown and overexpression of MIF mutants which are resistant to shRNA1 and 3 in cortical neurons. (H) Quantification of NMDA-induced neuronal cell death in MIF knockdown cortical neurons and cells overexpressing MIF mutants, which are resistant to shRNA1 and 3. Means±SEM are shown. ***P<0.001, versus CSS control. ###P<0.001, versus WT with NMDA treatment, one-way ANOVA. Scale bar, 100 μm. Intensity of MIF signal is shown in C, F & H. The experiments were repeated in three independent trials.

FIGS. 14A-14O. FIG. 14. MIF contains PD-D/E(x)K nuclease motif. (A) Alignments of the nuclease domains of MIF from human, mouse, rat, monkey, pig, bovine, sheep, rabbit and Sorex. (B) Alignments of the CxxCxxHx(n)C domain of MIF from human, mouse, rat, monkey, pig, bovine, sheep, rabbit, and Sorex. (C) Conserved topology of the active site in PD-D/E(x)K nucleases. Image modified from Kosinski et al., (18). The alpha helices are shown as circles and beta strands are shown as triangles. The orientations of the beta-strands indicate parallel or antiparallel. (D) Crystal structure of MIF trimer (pdb:1GD0). Each monomer is indicated by a different color. (E) Topology of MIF trimer illustrating the orientations of the various domains similar to PD-D/E(x)K motif. (F) Crystal structure of the MIF monomer containing the PD-D/E(x)K domain derived from the trimer (broken red line in D) by hiding two of the monomers. (G) Topology of a MIF monomer in the MIF trimer. (H) Illustrating each monomer has a PD-D/E(x)K domain. The PD-D/E(x)K motif is made of two parallel β-strands (β4 and β5) from one monomer and two anti-parallel strands (β6 and β7) from the adjacent monomer. (I) A schematic diagram of the similarity in topology of the MIF monomer in the MIF trimer and EcoRV illustrating similar orientations of the various domains in their nuclease domains. The alpha helices are shown as circles and beta strands are shown as triangles. (J) Topology of EcoRV monomer. (K) Alignment of MIF monomer in the MIF trimer and EcoRV monomer (red). (L-O) Alignments of PD-D/E(x)K motif in MIF and other well-known nucleases including EcoRI (magenta, pdb: 1QC9), EcoRV (light blue, pdb: 1SX8), ExoIII (red, pdb: 1AKO), and PvuII (orange, pdb 1PVU). All five motifs show similar orientations of the four beta strands in the beta-sheet against the alpha helices as observed in a typical PD-D/E(x)K motif active site.

FIGS. 15A-15H. FIG. 15. MIF is a novel nuclease. (A) Concentration-dependence of MIF incubation with human genomic DNA (hgDNA, 200 ng) in Tris-HCl buffer pH 7.0 containing 10 mM MgCl₂ at 37° C. for 4 hrs. (B) Time course of MIF incubation (4 μM) with hgDNA in the Tris-HCl buffer pH 7.0 containing 10 mM MgCl₂ at 37° C. (C) MIF (8 μM) incubation with hgDNA in the Tris-HCl pH 7.0 buffer with different ions as indicated at 37° C. for 4 hrs. (D) In vitro pulse-field gel electrophoresis-nuclease assay with purified proteins (4 μM) using human genomic DNA as the substrate. (E) Different purified MIF mutants (see FIG. 1D for illustration of MIF's amino acid sequence) were incubated with hgDNA in the Tris-HCl buffer pH7.0 containing 10 mM MgCl₂ at 37° C. for 4 hrs. Coomassie blue staining of purified MIF WT protein and MIF mutants are shown (lower panel). (F) The glutamate residue was mutated into Glutamine, Aspartate and Alanine. (G) Coomassie blue staining of purified MIF WT protein and MIF mutants. (H) Different purified MIF mutants (see FIG. 1D for illustration of mutations) were incubated with hgDNA in the Tris-HCl buffer pH 7.0 containing 10 mM MgCl₂ at 37° C. for 4 hrs. Coomassie blue staining of purified MIF WT protein and MIF mutants are shown (lower panel). The experiments were repeated using MIF protein purified in three independent preparations.

FIGS. 16A-16M. FIG. 16. Effects of MIF mutation on protein folding and enzyme activities. (A) Oxidoreductase activity of MIF proteins. (B) Tautomerase activity of MIF proteins. Means±SEM are shown in B and C. **P<0.01, one-way ANOVA. (C) The FPLC profile of MIF proteins (wild type, E22Q and E22A) (solid line) and protein standard (broken line). (D) Coomassie blue staining of MIF fractions from the FPLC. (E-M) UV-CD analyses of purified MIF recombinant proteins in presence and absence of magnesium chloride (Mg) and/or zinc chloride (Zn). The experiments were replicated three times using MIF purified from three independent preparations.

FIGS. 17A-17G. FIG. 17. Characterization of MIF-DNA binding by ChIP-seq. (A) Sonicated fragments of chromatin are in the range of 100-200 bp for ChIP-seq in the DMSO and MNNG treated cells. (B) Representative immunoblot images of MIF ChIP. (C) Number and coverage of the reads from four different libraries including DNA inputs and MIF ChIP samples prepared from DMSO or MNNG (50 μM) treated cells. (D) MIF ChIP-peak distribution across different genomic regions in MNNG treated cells. The pie chart shows that MIF tends to bind to promoters of genes (about 36% of ChTP regions are in promoters). (E-F) Representative IGV visualization of MIF enrichment on the genome shown in two different chromosome window sizes. The top two lines show the tdf file of ChIP-seq data from DMSO and MNNG treated cells. The third and fourth lines show the bed files for DMSO and MNNG treated samples. The peaks were only observed in MNNG treated samples, but not in DMSO treated samples. The last line indicates the hgl9 reference genes. (G) MIF chromatin enrichment in DMSO and MNNG treated cells confirmed by qPCR with Non-P (non peak regions), P55101, P66005, P65892, P36229, P46426 and P62750 (peak regions).

FIGS. 18A-18C. FIG. 18. MIF binds to single stranded DNA. (A) Alignment of MIF DNA binding motif. (B) Images of MIF trimer (PDB accession 1FIM) surface showing a groove/binding pocket (arrows) (Top panel). Models of MIF trimer with dsDNA in the groove (Middle panel). Right image in the middle panel shows the side view of the overlay of MIF-dsDNA (PDB accession 1BNA) with MIF-ssDNA (PDB accession 2RPD) models. i-iii, Cartoon images showing residues P16 and D17 close to dsDNA and ssDNA whereas E22 is close to the ssDNA but not the dsDNA. (C) EMSA demonstrating that MIF binds to its single strand 5′ biotin-labeled DNA binding motif (PS³⁰) in the presence or absence of Mg²⁺ or unlabeled PS³⁰. MIF binding to its DNA substrate is disrupted by a MIF antibody, whereas MIF mutants E22A, E22Q, P16A, D17A, D17Q still bind to its DNA substrate. Experiments were replicated for four times using MIF protein purified from three independent preparations.

FIG. 19. Secondary structures of different biotin-labeled DNA substrates used in binding and cleavage assays.

FIGS. 20A-20H. FIG. 20. MIF cleaves stem loop ssDNA with structure-specific nuclease activity. (A) MIF nuclease assay using dsPS¹⁰⁰ as substrate. (B) MIF nuclease assay using ssPS¹⁰⁰ and its complementary strand ssPS^{100R} as substrates. (C) MIF (1-4 μM) has no obvious nuclease activity on double strand DNA using dsPS³⁰, its sequence related substrate-dsRF and non-related substrate-dsL3. (D) MIF (0.5-4 μM) fails to cleave dsPS³⁰, dsRF, dsL3 in a concentration-dependent manner. (E) Mg²⁺ is required for MIF nuclease activity using ssPS³⁰ as substrate. (F-H) MIF (2 μM) cleaves ssPS³⁰ in a concentration- and time-dependent manner.

FIGS. 21A-21J. FIG. 21. MIF interacts with AIF and cotranslocates to the nucleus. (A) Schematic representation of the GST-AIF truncated proteins used in the binding assays. (B) GST pull-down assays visualized by western blot using an anti-MIF antibody (upper panel). Coomassie blue staining of GST fusion AIF truncated proteins used in the pull-down experiments (lower panel). (C) Pull-down assay of AIF mutants visualized by western blot using an anti-MIF antibody. (D) GST-MIF and its variants on glutathione beads pulled down AIF protein. The experiments were replicated in three independent trials. (E-G) Nuclear translocation of AIF and MIF after MNNG treatment in the presence or absence the PARP inhibitor, DPQ (30 μM) in HeLa cells, which was determined by (E) immunostaining and (F-G) subcellular fractionation. Scale bar, 20 μm. The experiments were replicated in three independent trials. ***P<0.001, one-way ANOVA. (H-J) Nuclear translocation of AIF and MIF after NMDA treatment in the presence or absence PARP inhibitor DPQ or nNOS inhibitor nitro-arginine (N-Arg, 100 μM) in cortical neurons, which was determined by subcellular fractionation. Intensity of MIF and AIF signal is shown in I & J. The experiments were replicated in three independent trials. The experiments were replicated in three independent trials. ***P<0.001, one-way ANOVA.

FIGS. 22A-22D. FIG. 22. MIF nuclease activity is critical for NMDA-induced DNA damage and PARP-1 dependent cell death in cortical neurons. (A) Representative images of NMDA-induced cytotoxicity in MIF WT, KO and lentivirus-transduced MIF KO cortical neurons expressing MIF WT, E22Q or E22A. Scale bar, 200 μm. (B-D) Quantification of NMDA-caused DNA damage 6 h after the treatment determined by comet assay. % of (B) tail positive neurons, (C) tail length and (D) % of DNA in tail.

FIGS. 23A-23D. FIG. 23. MIF is critical for MNNG-induced DNA damage in HeLa Cells. (A) Representative images of MNNG-caused DNA damage determined by the comet assay in WT HeLa cells, NT shRNA or MIF shRNA lentivirus-transduced HeLa cells. Dashed lines indicate the center of the head and tail. Scale bar, 20 μm. (B-D) Quantification of (B) % of tail positive cells, (C) tail length and (D) % of DNA in tail. Means±SEM are shown in b-d. ***P<0.001, ###P<0.001, one-way ANOVA. The experiments were replicated in three independent trials.

FIGS. 24A-24H. FIG. 24. MIF nuclease activity is required for parthanatos in stroke in vivo. (A) Intracerebroventricular (ICV) injection with trypan blue dye. (B) Representative immunostaining images of expression of AAV2-MIF WT in (i) cortex, (ii) striatum and (iii & iv) hippocampus 79 days after injection. Scale bar, 50 μm. (C) Laser-Doppler flux measured over the lateral parietal cortex in the core of the ischemic region in WT (n=16), MIF KO (n=12), MIF KO-WT (n=11), MIF KO-E22Q (n=11) and MIF KO-E22A (n=11) mice. (D-E) Quantification of infarction volume in cortex, striatum and hemisphere 1 day or 7 days after 45 min MCAO. (F) Neurological deficit was evaluated by % of right turns in the corner test 1, 3 and 7 days after 45 min MCAO surgery. WT MCAO (n=16), KO MCAO (n=12), KO-WT MCAO (n=16). KO-E22Q MCAO (n=16), KO-E22A MCAO (n=16). Means±SEM are shown. *P<0.05, versus pre stroke control, one-way ANOVA. (G) Nuclear translocation of AIF (red) and MIF (green) and (H) DNA fragmentation as determined by pulse field gel electrophoresis in the penumbra after MCAO in MIF WT, KO and KO mice, which were injected with AAV2-MIF WT, E22Q or E22A 1 day, 3 days or 7 days after MCAO surgery. WT MCAO (n=29), KO MCAO (n=20), KO-WT MCAO (n=23). KO-E22Q MCAO (n=22), KO-E22A MCAO (n=19). Means±SEM are shown in D-F. *P<0.05 (E), ***P<0.001 (D, F), versus control or baseline, one-way ANOVA.

### DETAILED DESCRIPTION OF THE INVENTION

The present invention is based on the identification of macrophage migration inhibitory factor (MIF) as a PARP-1 dependent AIF-associated nuclease (PAAN).

As used herein, a “therapeutically effective amount” of a compound, is intended to qualify the amount of active ingredients used in the treatment of a disease or disorder. This amount will achieve the goal of reducing or eliminating the said disease or disorder. The exact dosage and frequency of administration depends on the particular compound of the invention used, the particular condition being treated, the severity of the condition being treated, the age, weight and general physical condition of the particular subject as well as the other medication, the patient may be taking, as is well known to those skilled in the art. Furthermore, said “therapeutically effective amount” may be lowered or increased depending on the response of the treated subject and/or depending on the evaluation of the physician prescribing the compounds of the instant invention.

As used herein, reference to “treating” or “treatment” of a subject is intended to include prophylaxis. The term “subject” means all mammals including humans. Examples of subjects include humans, cows, dogs, cats, goats, sheep, pigs, and rabbits. Preferably, the subject is a human.

In addition to invention compounds, one of skill in the art would recognize that other therapeutic compounds including chemotherapeutic agents, anti-inflammatory agents, and therapeutic antibodies can be used prior to, simultaneously with or following treatment with invention compounds. While not wanting to be limiting, chemotherapeutic agents include antimetabolites, such as methotrexate, DNA cross-linking agents, such as cisplatin/carboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, or mechlorethamine. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like. Classes of anticancer agents suitable for use in compositions and methods of the present invention include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-alpha, etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis.

Examples of other therapeutic agents include the following: cyclosporins (e.g., cyclosporin A), CTLA4-Ig, antibodies such as ICAM-3, anti-TL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39 (i.e., CD154), fusion proteins constructed from CD40 and gp39 (CD40Ig and CD8 gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-α inhibitors such as tenidap, anti-TNF antibodies or soluble TNF receptor, and rapamycin (sirolimus or Rapamune) or derivatives thereof.

Other agents that may be administered in combination with invention compositions and methods including protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay.

The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

Inhibition or genetic deletion of poly(ADP-ribose) polymerase-1 (PARP-1) is profoundly protective against a number of toxic insults in many organ systems. The molecular mechanisms underlying PARP-1-dependent cell death involve mitochondrial apoptosis-inducing factor (AIF) release and translocation to the nucleus resulting in chromatinolysis. How AIF induces chromatinolysis and cell death is not known. The present invention identifies Macrophage Migration Inhibitory Factor (MIF) as a PARP-1 dependent AIF-associated nuclease (PAAN) that possesses Mg²⁺/Ca²⁺-dependent nuclease activity. AIF is required for recruitment of MIF to the nucleus where MIF cleaves genomic DNA into 20-50 kb fragments. Depletion of MIF, disruption of the AIF-MIF interaction or mutation of E22 to Q22 in the catalytic nuclease domain blocks MIF nuclease activity, inhibits chromatinolysis and cell death following glutamate excitotoxicity in neuronal cultures and focal stroke in mice. Inhibition of MIF's nuclease activity is a potential critical therapeutic target for diseases that are due to excessive PARP-1 activation.

MIF is thought to be required for PARP-1 dependent cell death induced by MNNG or NMDA excitotoxicity.

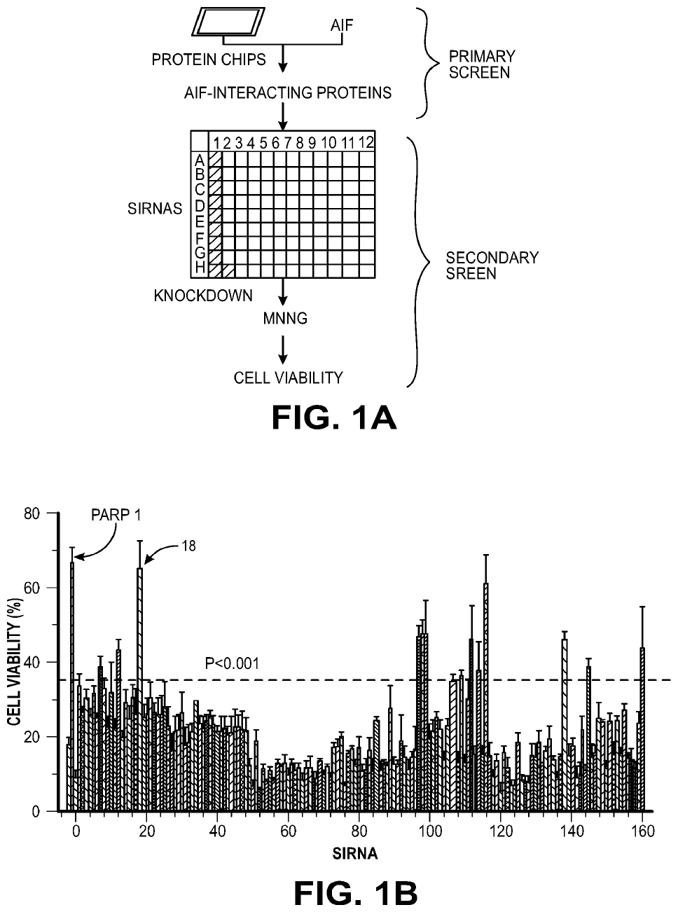

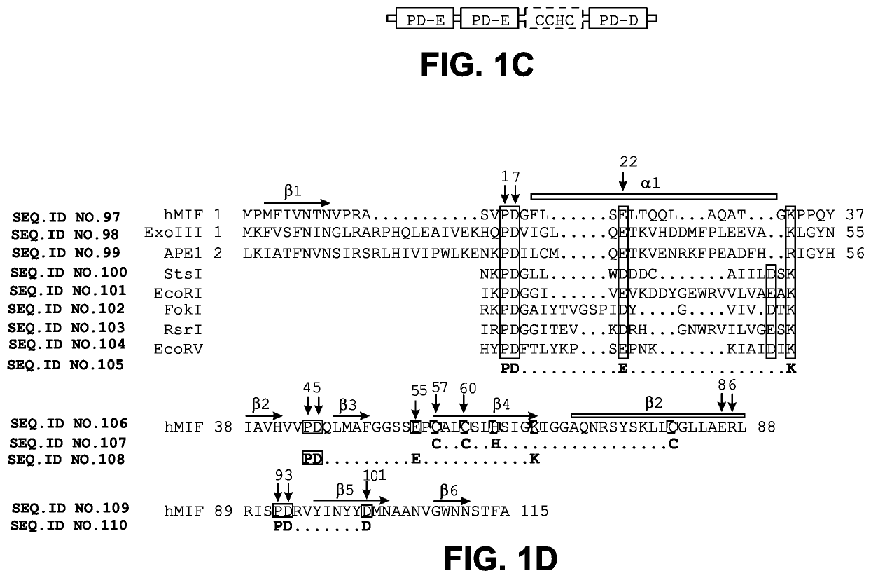

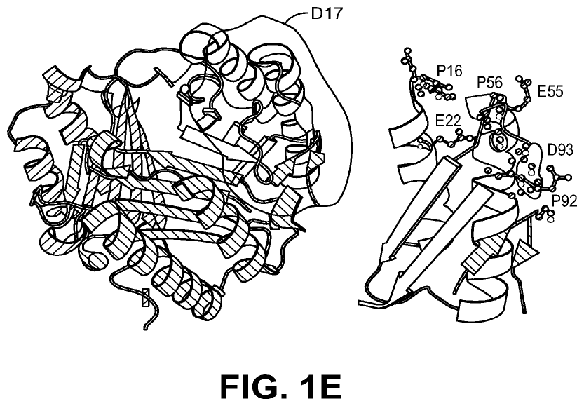

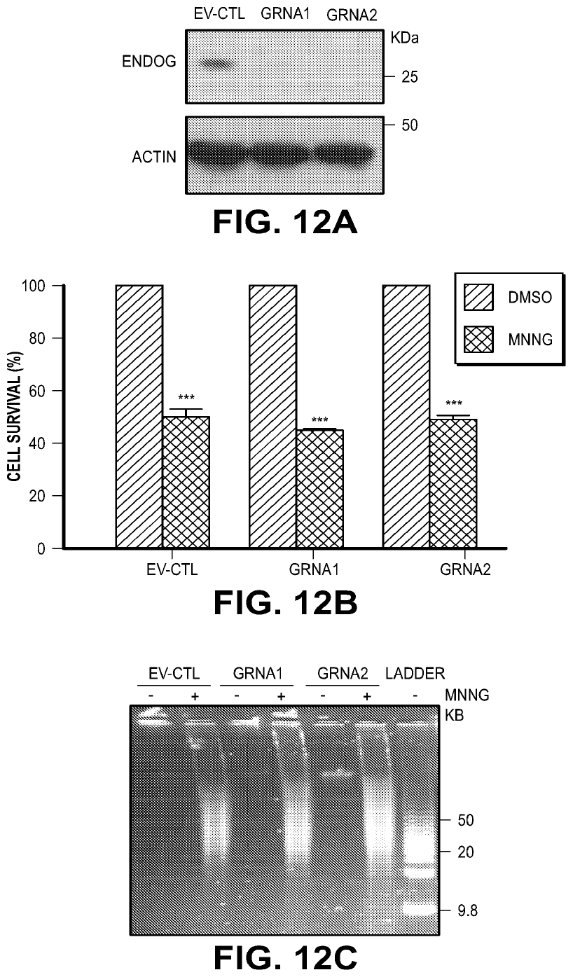

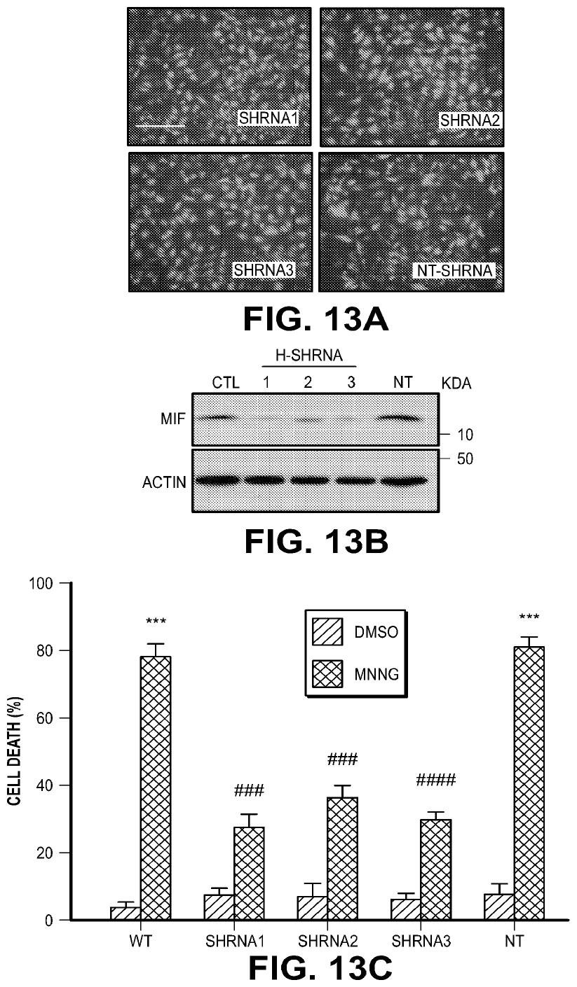

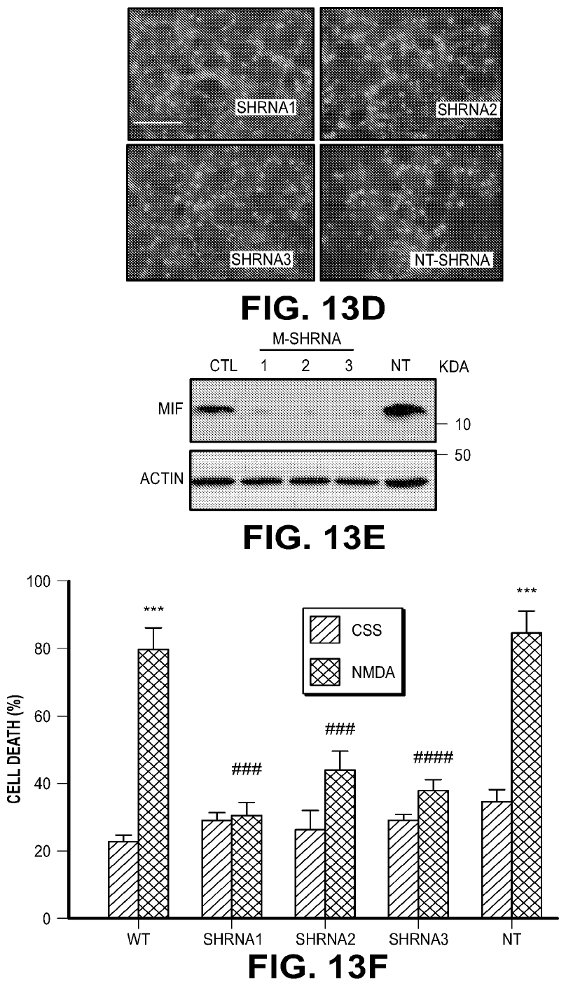

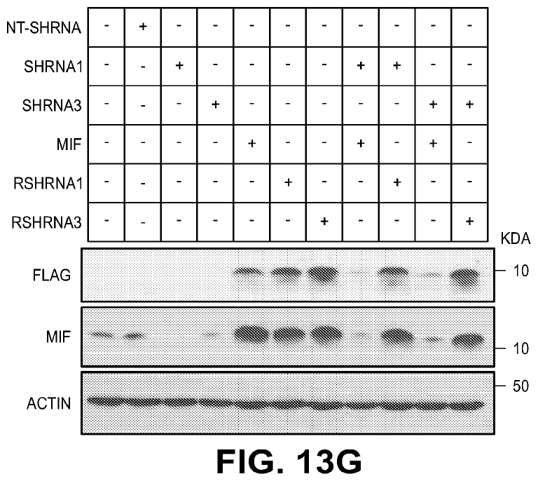

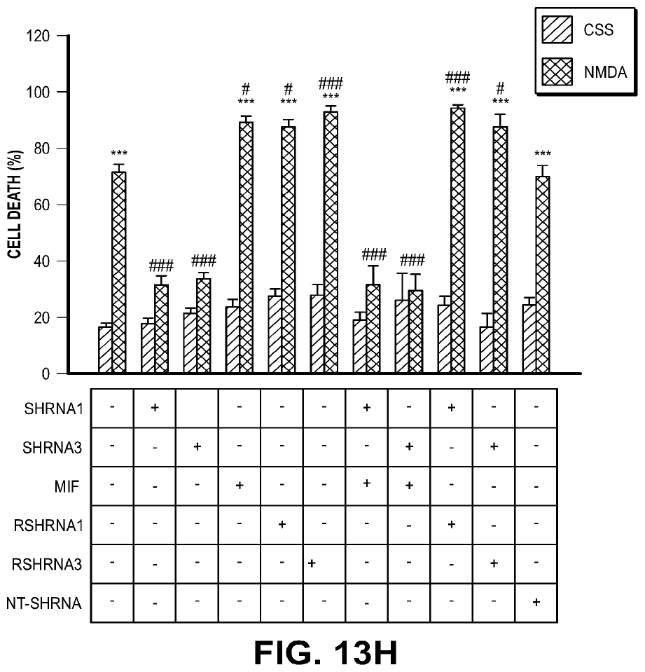

Consistent with the previous findings (13, 14), EndoG is dispensable for PARP-1 dependent large DNA fragmentation and MNNG induced cell death (FIG. 12). To identify a PARP-1 dependent AIF Associated Nuclease (PAAN), 16K and 5K protein chips (15) were probed with recombinant AIF. The strongest 160 AIF interactors were advanced to a siRNA based screen to identify modifiers of parthanatos induced by MNNG in HeLa cell culture, a well characterized method to study parthanatos (2, 11, 12) (FIGS. 1, A and B). These AIF interactors were further segregated based on the ability of their knockdown to provide protection equivalent to knockdown of PARP-1 and whether they exhibited sequence and structure homology consistent with possible nuclease activity. It was found that knockdown of AIF interactor 18 is as protective as PARP-1 knockdown (FIG. 1i). AIF interactor 18, is previously known under a variety of synonyms and it is collectively known as macrophage migration inhibitory factor (MIF or MMIF) (16, 17). Three different shRNA constructs against human and mouse MIF were utilized to confirm that knockdown of MIF protects against parthanatos induced by MNNG toxicity in HeLa cells or NMDA excitotoxicity in mouse primary cortical neurons (FIG. 13, A to F). To rule out off-target effects from the shRNA, MIF constructs that are resistant to shRNA 1 (RshRNA1) and 3 (RshRNA3) were made and shown to be impervious to knockdown (FIG. 13G). These resistant MIF constructs restore NMDA excitotoxicity in the setting of endogenous MIF knockdown (FIG. 13H) confirming that MIF is required for parthanatos induced by MNNG or NMDA.

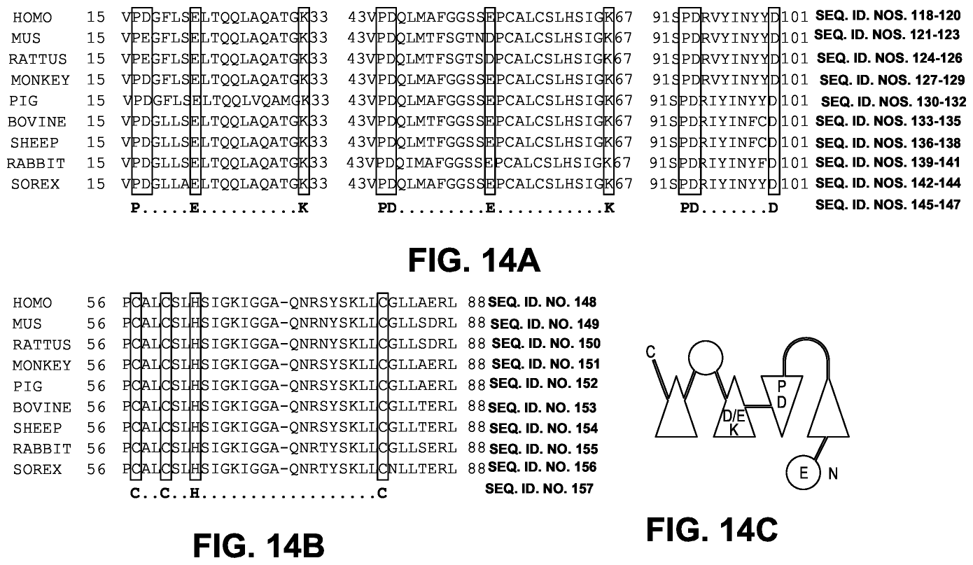

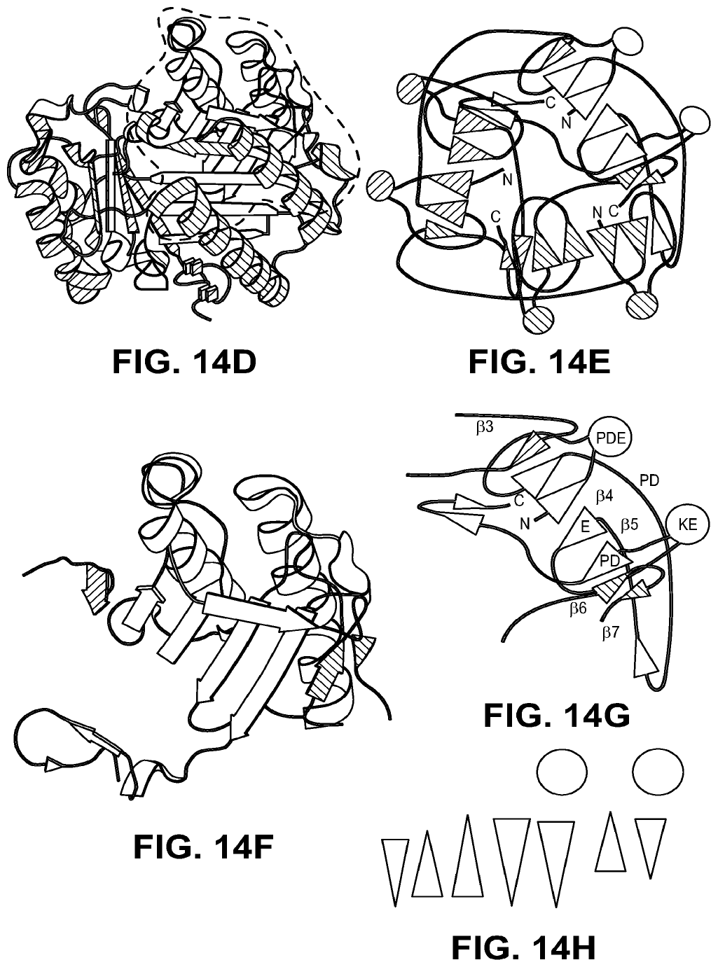

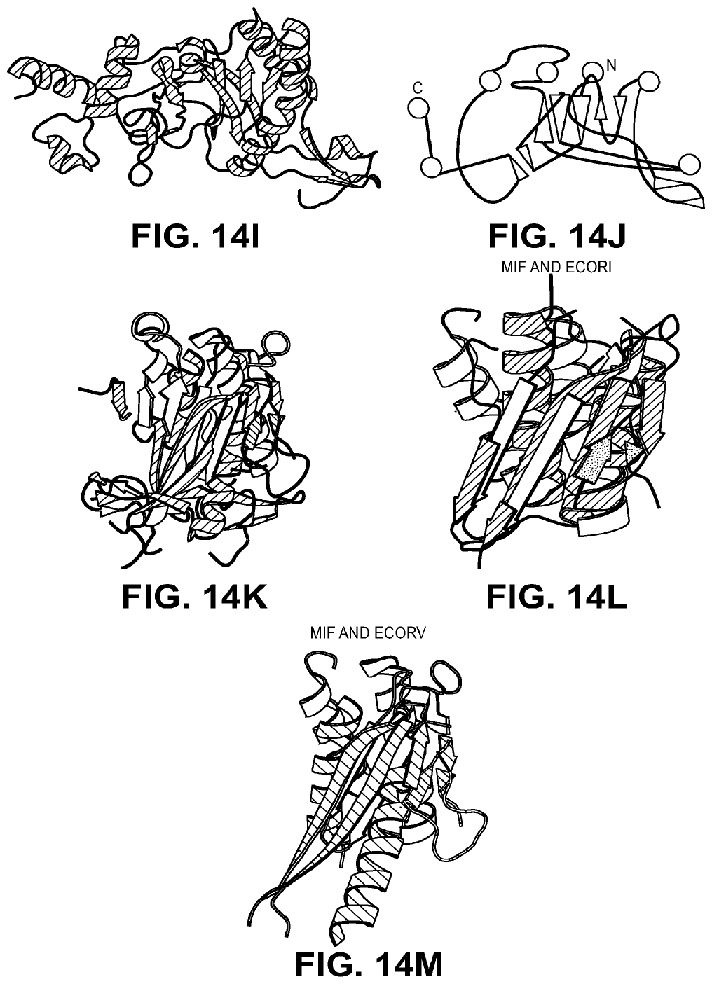

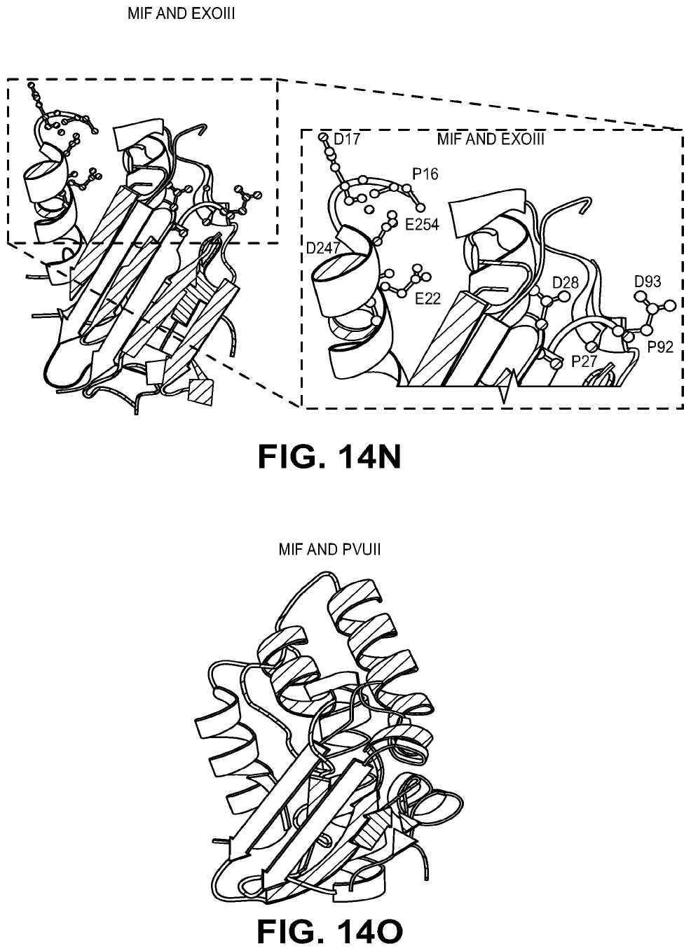

MIF contains three PD-D/E(X)K motifs that are found in many nucleases (18-20) (FIGS. 1, C and D) and are highly conserved across mammalian species (FIG. 14A). In addition, it contains a CxxCxxHx₍ₙ₎C zinc finger domain (FIG. 1C and FIG. 14B), which is commonly found in DNA damage response proteins (20). MIF is known to exist as a trimer (21-23). The core PD-D/E(X)K topology structure in the MIF trimer consists of 40-strands next to 2α-strands (FIG. 1E and FIG. 14, C to G), which is similar to those of well characterized nucleases including EcoRI, EcoRV, ExoIII and PvuII (FIG. 14, H to O). These sequence analysis and 3-D modeling results indicate that MIF belongs to the PD-D/E(X)K nuclease-like superfamily (24, 25).

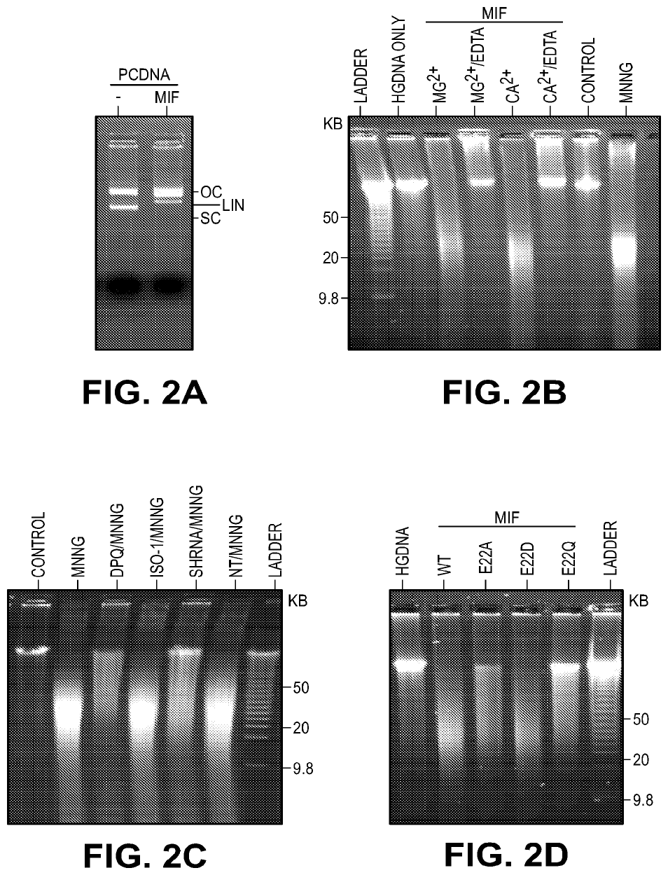

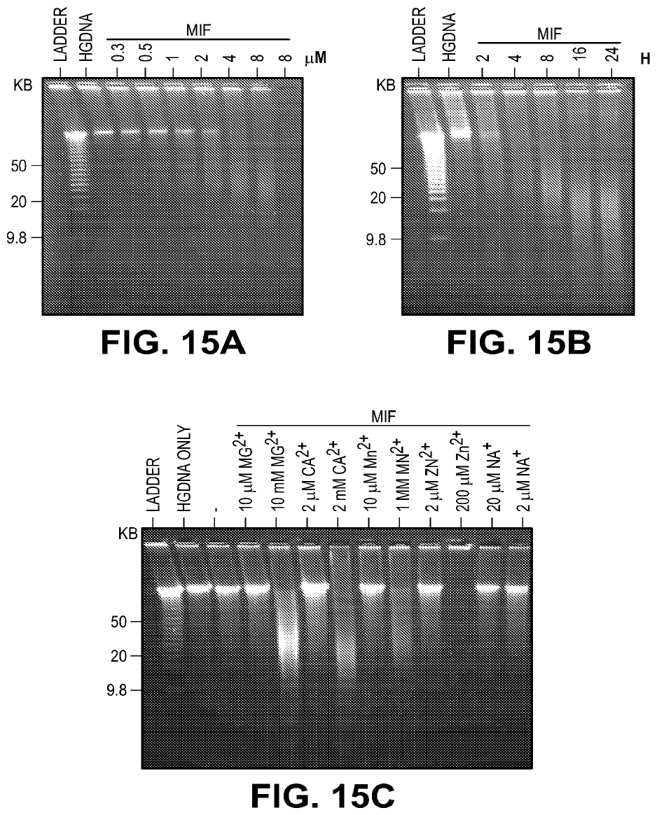

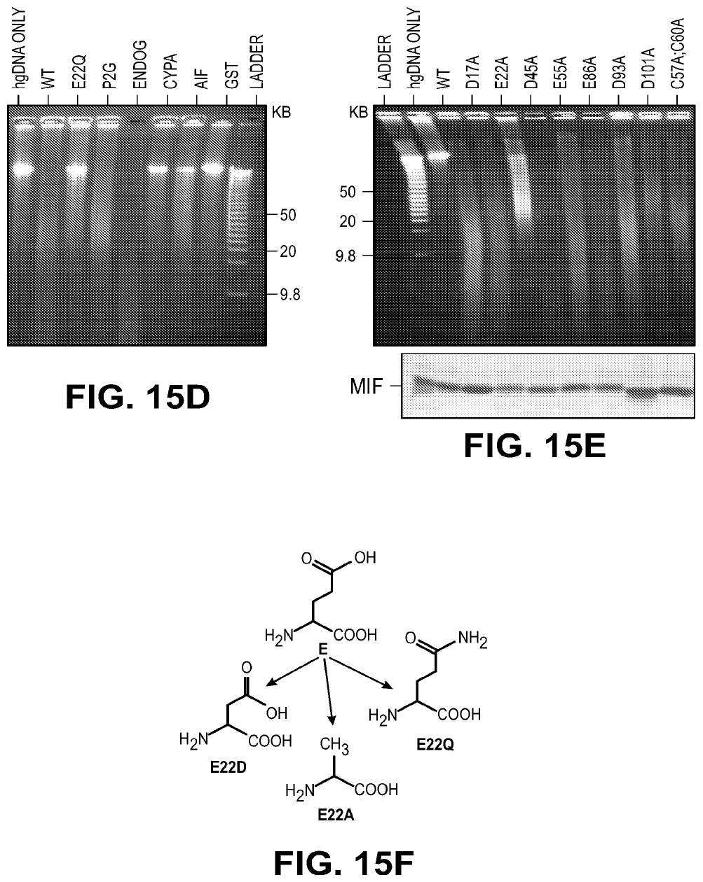

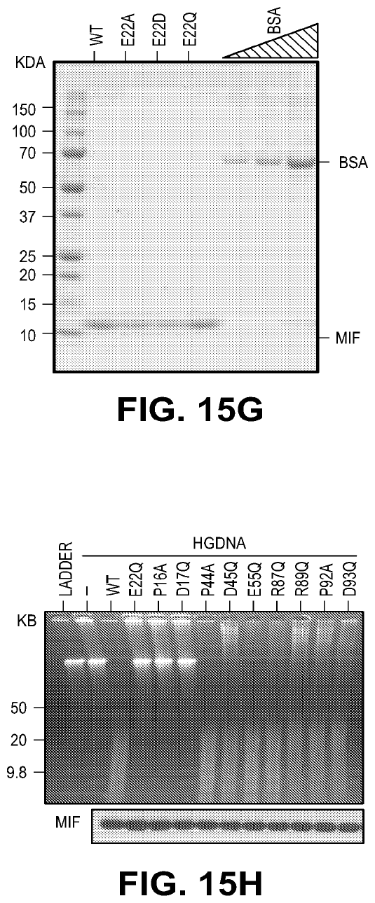

To determine if MIF has nuclease activity, pcDNA plasmid was incubated together with recombinant MIF. Supercoiled pcDNA is cleaved by MIF into an open circular form and further cleaves it into a linear form (FIG. 2A). Moreover, MIF cleaves human genomic DNA in a concentration and time dependent manner (FIGS. 15, A and B). Addition of 10 mM Mg²⁺, 2 mM Ca²⁺, or 1 mM Mn²⁺ is required for MIF nuclease activity (FIG. 15C) consistent with the divalent cation concentrations required for in vitro activity of other similar nucleases (26). EDTA blocks MIF's nuclease activity against human genomic DNA (FIG. 2B). In the absence of the divalent cation or with the cation at 2-10 μM, MIF has no nuclease activity (FIG. 15C). Addition of 200 μM Zn²⁺ precipitates genomic DNA in the presence of MIF while 2 μM Zn²⁺ has no effect. In addition, Na⁺ has no effect on MIF's nuclease activity (FIG. 15C). Importantly, pulse-field gel electrophoresis indicates that MIF cleaves human genomic DNA into large fragments comparable to the DNA purified from HeLa cells treated with MNNG (FIG. 2B, lane 8). shRNA knockdown of MIF prevents MNNG induced DNA cleavage, which is similar to the effect of PARP inhibition by 3,4-dihydro-5[4-(1-piperindinyl)butoxy]-1(2H)-isoquinoline (DPQ) (FIG. 2C). Since MIF has been touted to possess tautomerase activity, the MIF tautomerase inhibitor, ISO-1 was examined (27). ISO-1 fails to prevent MNNG induced DNA damage (FIG. 2C). Moreover, the MIF P2G tautomerase mutant, which lacks tautomerase activity (28), has no effect on MIF's nuclease activity (FIG. 15D). These data taken together indicate that MIF is a nuclease and it plays an important role in PARP-1 dependent DNA fragmentation.

To identify amino acid residues critical for MIF's nuclease activity, key aspartate, glutamate and proline residues within the PD-D/E(X)K domains of MIF were mutated. Substitution of glutamate 22 by alanine (E22A) or glutamine (E22Q), but not aspartate (E22D), clearly inhibits MIF's nuclease activity (FIG. 2D, FIG. 15, E to H). These data suggest that this glutamic acid residue (E22) in the first α-helix of MIF is critical for its nuclease activity, which is consistent with prior reports that this glutamic acid in the first α-helix of many Exonuclease-Endonuclease-Phosphatase (EEP) domain superfamily nucleases is highly conserved and it is the active site for nuclease activity (24, 25).

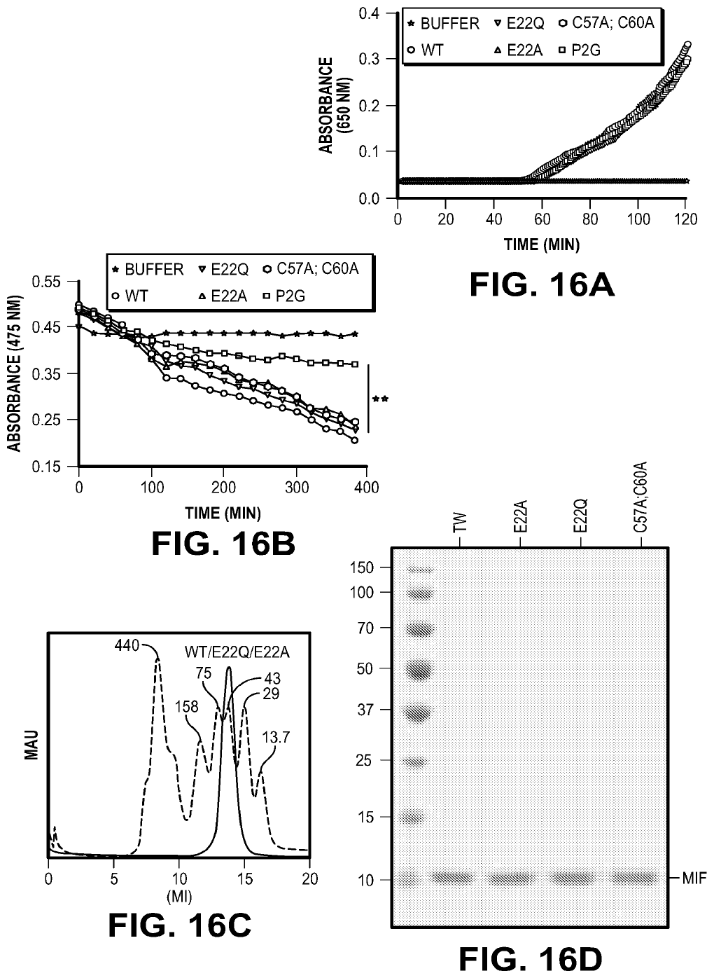

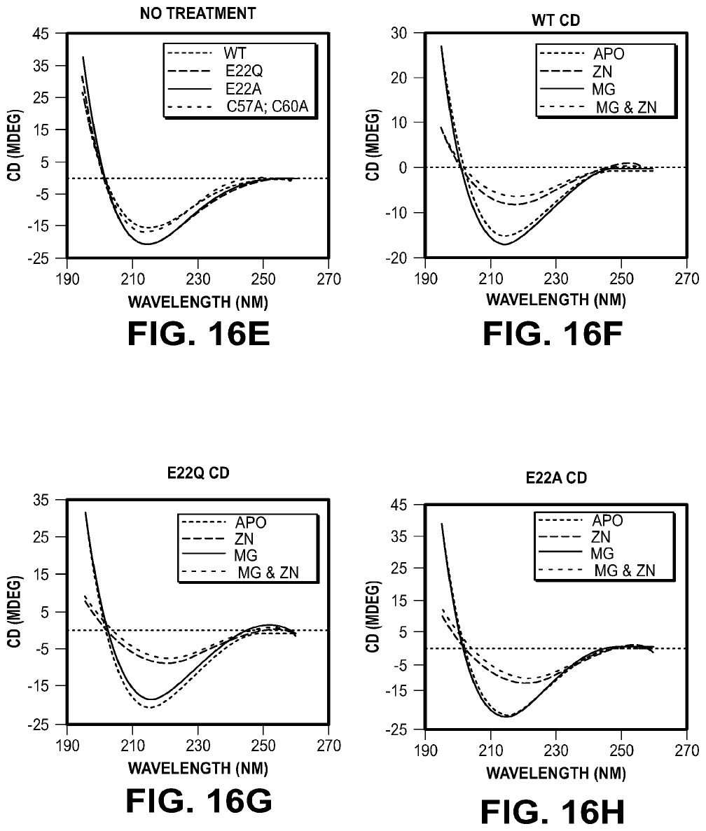

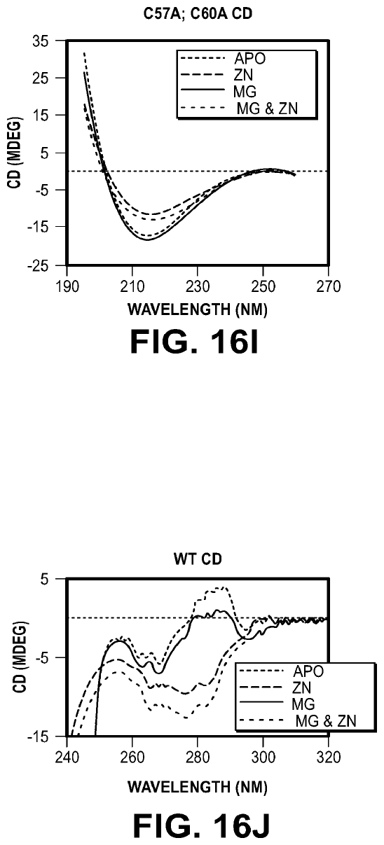

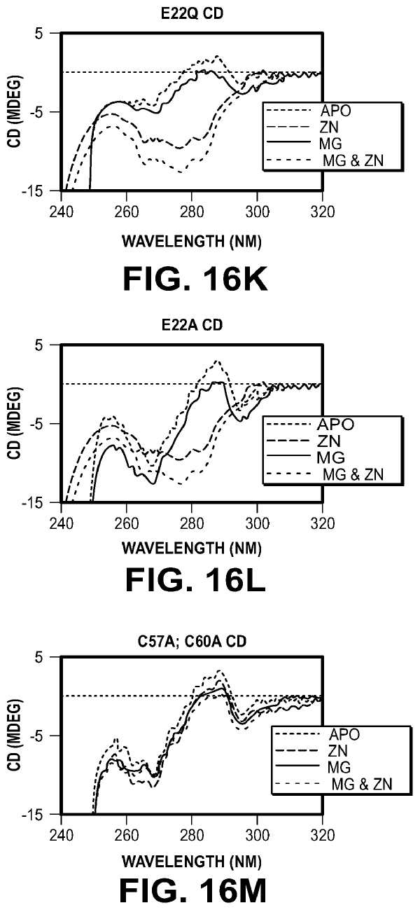

Previous studies indicate that MIF has both oxidoreductase and tautomerase activities (27, 29, 30). MIF active site mutants E22Q and E22A have no appreciable effect on MIF's oxidoreductase or tautomerase activities (FIGS. 16, A and B), suggesting that MIF nuclease activity is independent of its oxidoreductase and tautomerase activities. Moreover, it was found that MIF's protein confirmation is unaffected by the E22Q and E22A mutations as determined by far-ultraviolet (UV) circular dichroism (CD) and near UV CD spectroscopy, common methods to study protein secondary and tertiary structure, respectively (FIG. 16, C to M). The purity of MIF proteins was confirmed by Coomassie blue staining, FPLC and mass spectrometry (MS) assays (FIG. 15G, 16C, 16D, Material and Methods). No adventitious nuclease contamination was observed.

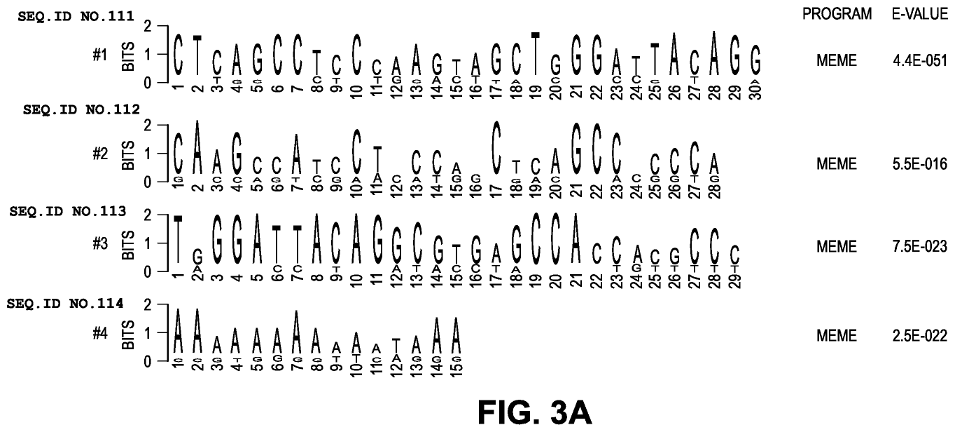

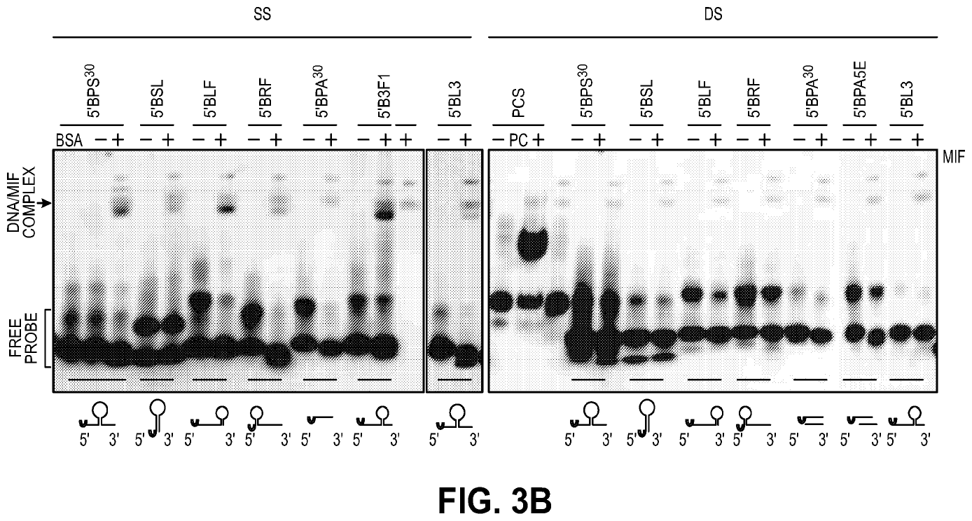

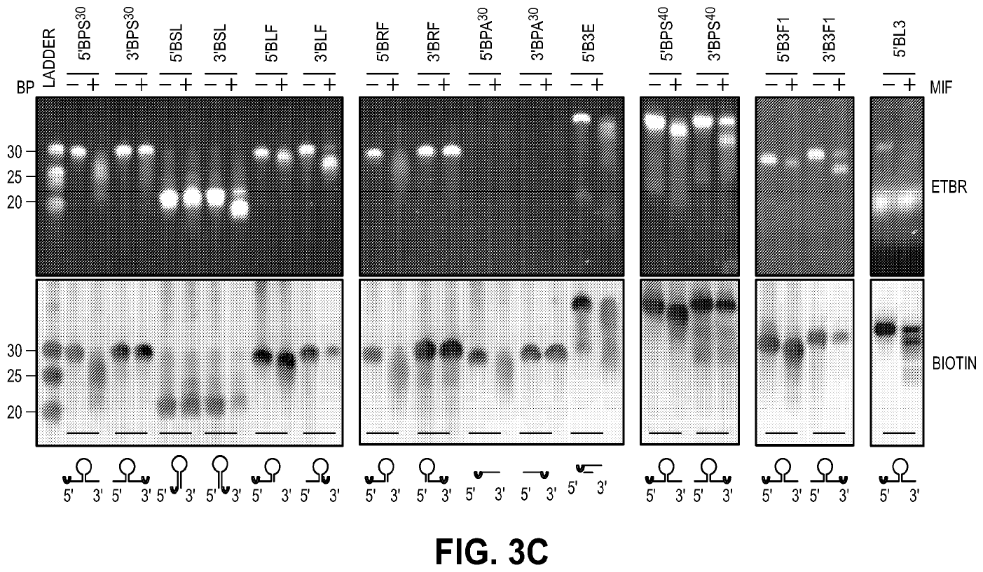

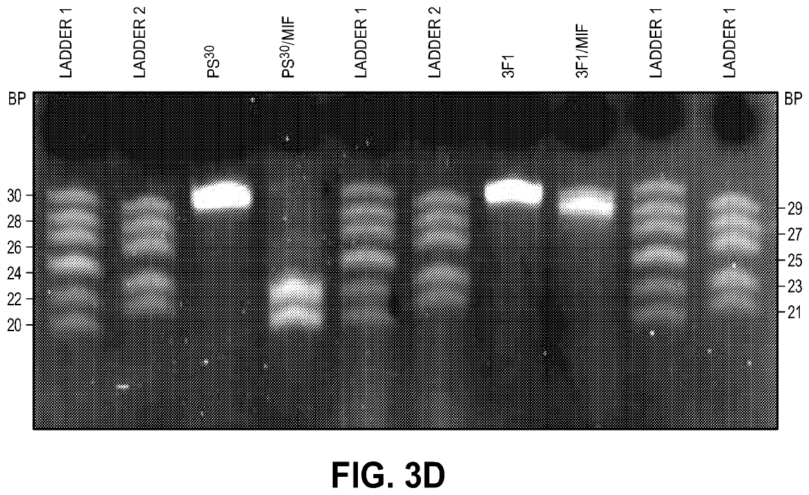

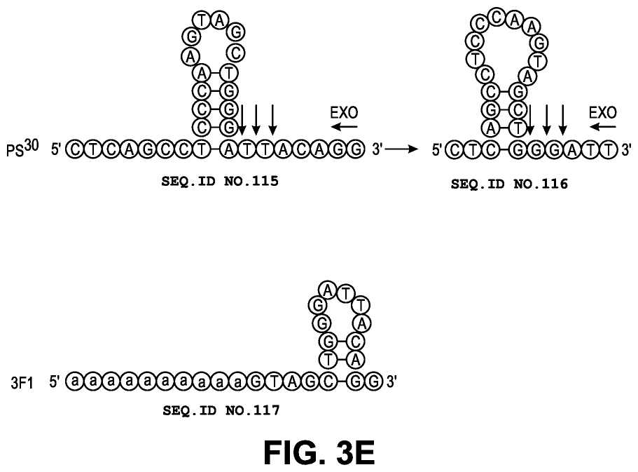

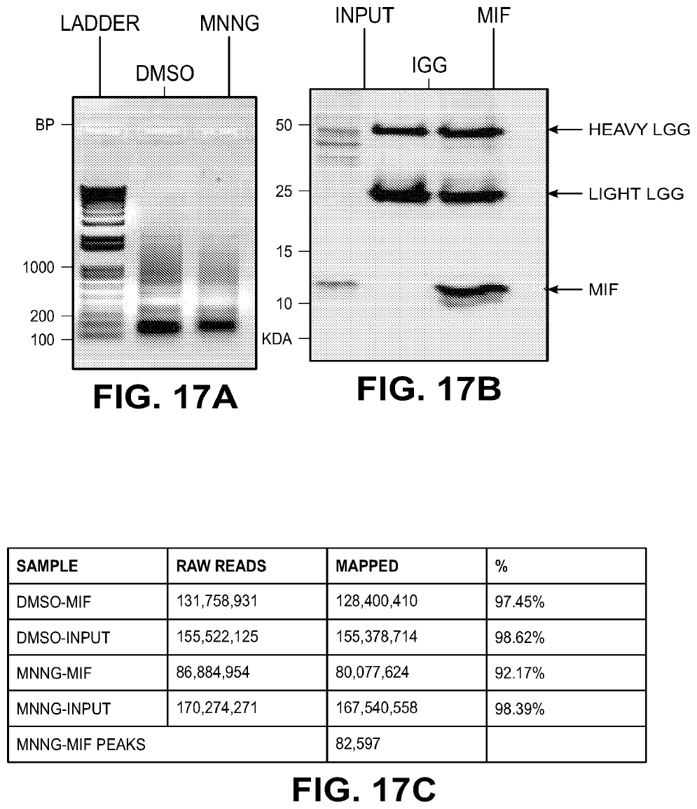

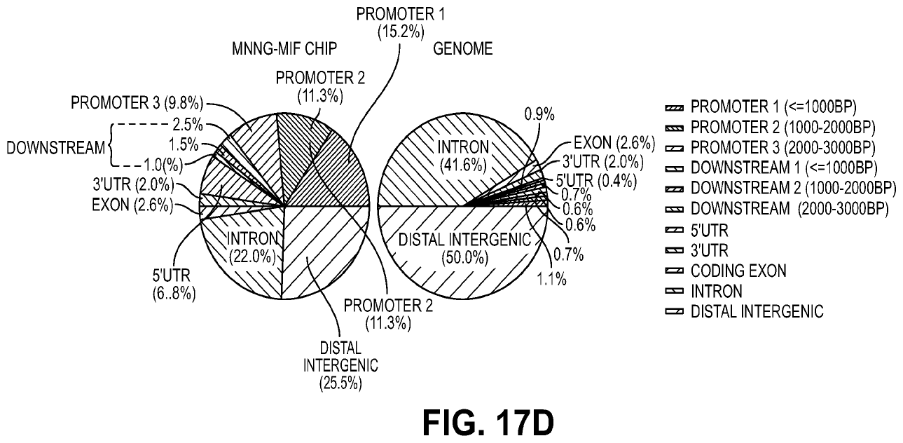

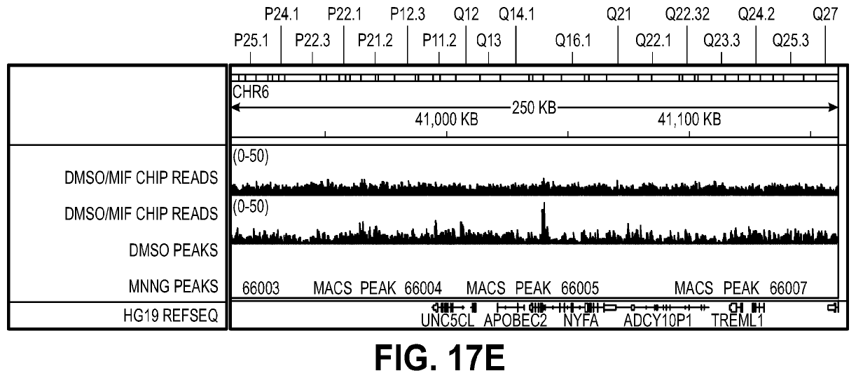

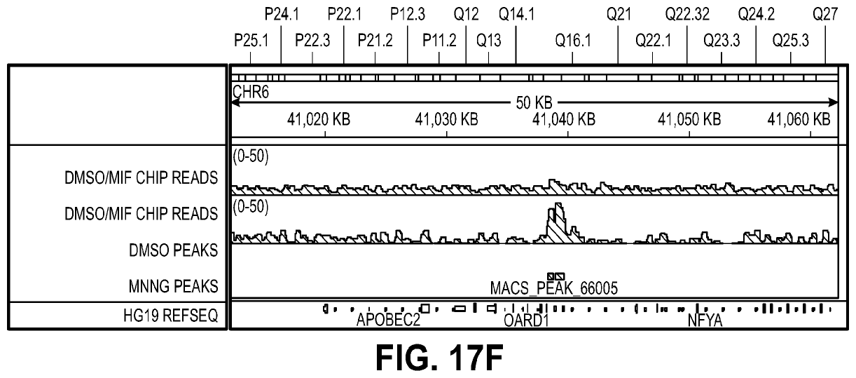

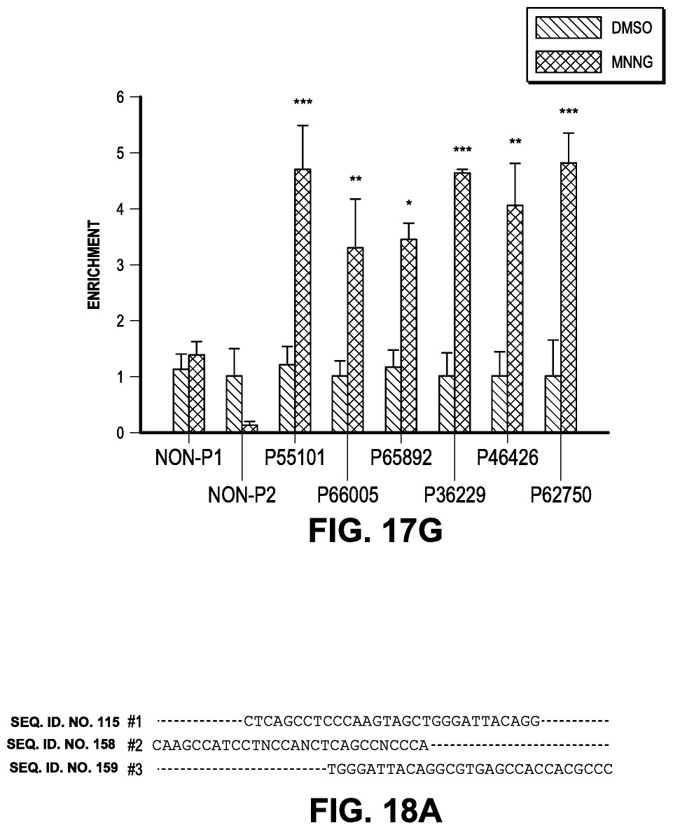

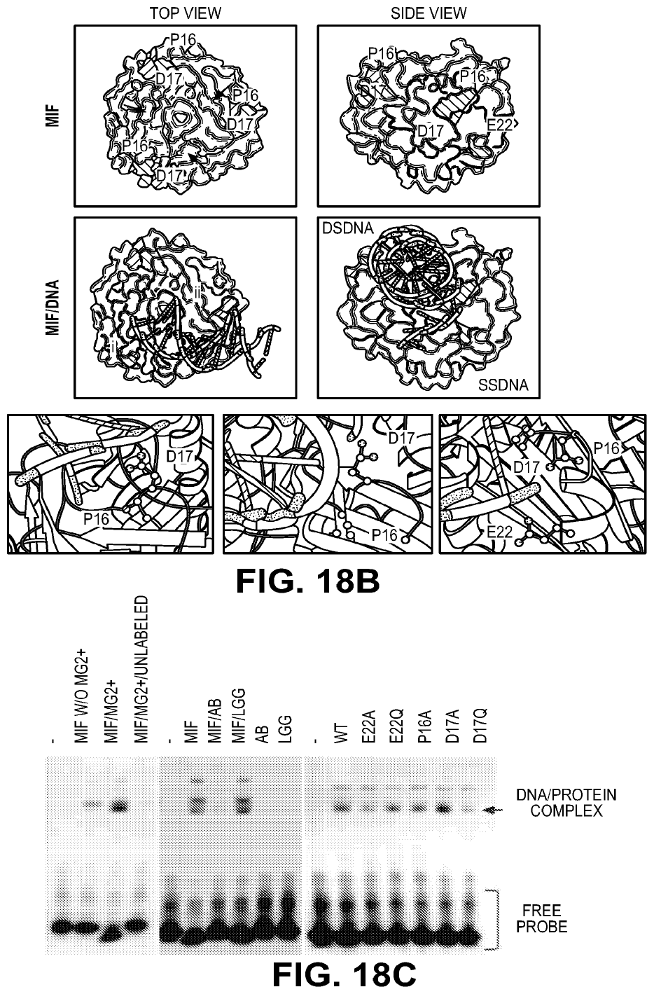

To further study whether MIF binds to DNA in HeLa cells treated with DMSO or MNNG (50 μM, 15 min), chromatin immunoprecipitation (ChIP) assays followed by deep sequencing were performed (FIG. 17). Using MEME-chip, two classes of MIF binding motifs (FIG. 3A) were identified. The first class (sequences 1-3) represents a highly related family of overlapping sequences (FIG. 3A and FIG. 18A). The sequence features of this family are best captured in sequence 1, the most statistically significant motif identified with 30 nucleotides and designated PS³⁰. The second class identified is a poly(A) stretch.

P16, D17 and E22 are within the same PD-D/E(X)K motif Three-dimensional computational modeling shows that P16 and D17 on MIF are close to double stranded DNA (dsDNA) whereas E22 is close to the ssDNA, indicating MIF might bind ssDNA or dsDNA or both (FIG. 18B). Both single stranded and double stranded forms of two classes of MIF DNA binding motifs were examined for MIF binding and cleavage specificity. The ssPS³⁰ sequence was synthesized with a 5′ biotin label and subjected to an electrophoretic mobility shift assay (EMSA) (FIG. 18C). It was found that MIF binds to the biotin labeled ssPS³⁰ forming one major complex in the presence of 10 mM Mg²⁺ (FIG. 18C), which is completely disrupted by the addition of excess unlabeled DNA substrate (PS³⁰) or a polyclonal antibody to MIF (FIG. 18C). MIF E22Q, E22A, P16A, P17A and P17Q mutants still form a MIF/ssPS³⁰ complex (FIG. 18C).

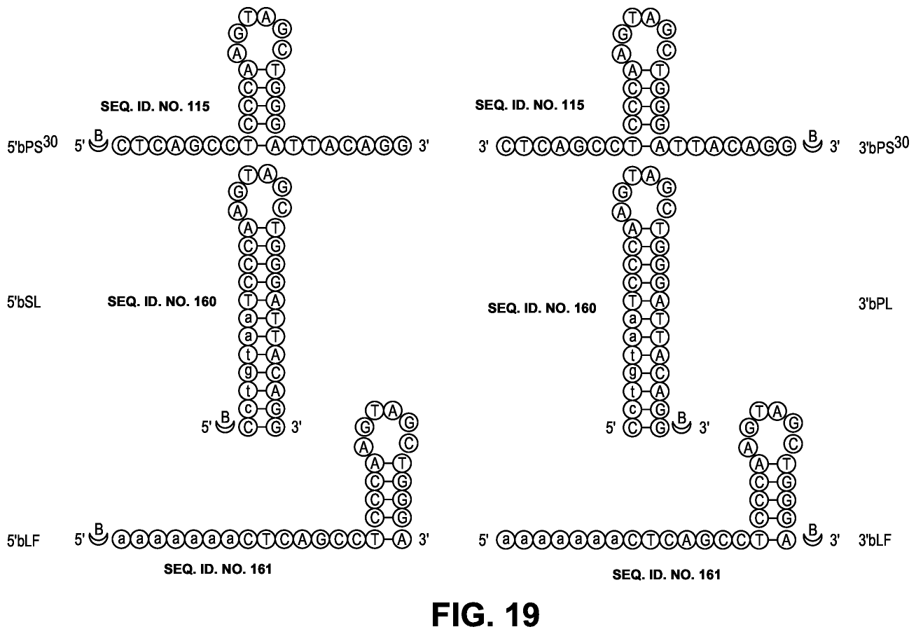

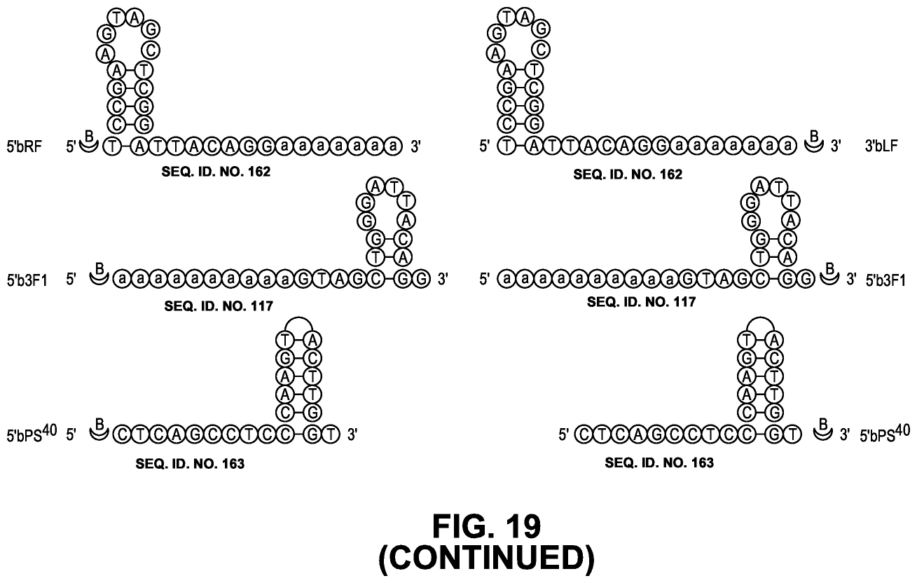

Since ssPS³⁰ has the potential to form a stem-loop structure with unpaired bases at the 5′ and 3′ ends, it was decided to determine if MIF binds to ssDNA with sequence or structure specificity. 5′ biotin labeled ssPS³⁰ and its sequence-related substrates with different structures by removing unpaired bases at the 5′ end, 3′ end, both 5′ and 3′ ends or eliminating the stem loop were used in the EMSA (FIG. 3B, and FIG. 19). It was found that completely removing the 3′ unpaired bases (5′bLF) has no obvious effect on the DNA/MIF complex formation (FIG. 2E and FIG. 19). In contrast, removing the 5′ unpaired bases (5′bRF) reduces the DNA-MIF binding, although MIF still binds to DNA with low efficiency. Similar results are observed when removing both 5′ and 3′ unpaired bases (5′bSL). These data suggest that MIF mainly binds to 5′ unpaired bases in ssDNA with stem loop structures. A poly A sequence that has no stem loop (5′bPA³⁰) and a short poly A sequence at the 5′ end of a stem loop structure (5′b3F1) were also used as the substrates and it was found that MIF fails to bind to 5′bPA³⁰, but clearly binds to 5′b3F1, suggesting that stem loop is required for MIF-ssDNA binding (FIG. 3B and FIG. 19). In addition to PS³⁰ sequence-related substrates, a non-sequence related substrate that has a stem loop like structure (5′bL3) was also tested and it was found that MIF weakly binds to 5′bL3. But its binding efficiency is much lower than that of 5′bPS³⁰. These data suggest that MIF preferentially binds to ssDNA with a stem loop and that it relies less on sequence specificity.

In parallel with the ssDNA studies, MIF was tested to see if it binds to dsDNA using PS³⁰, poly A, PS³⁰ sequence-related substrates (5′bPS³⁰, 5′bSL, 5′bLF, 5′bRF, 5′bPA³⁰, and 5′bPA5E) as well as non-related sequences (PCS and 5′bL3) (FIG. 3B and FIG. 19). It was found that MIF fails to bind to any of these double stranded substrates (FIG. 3B).

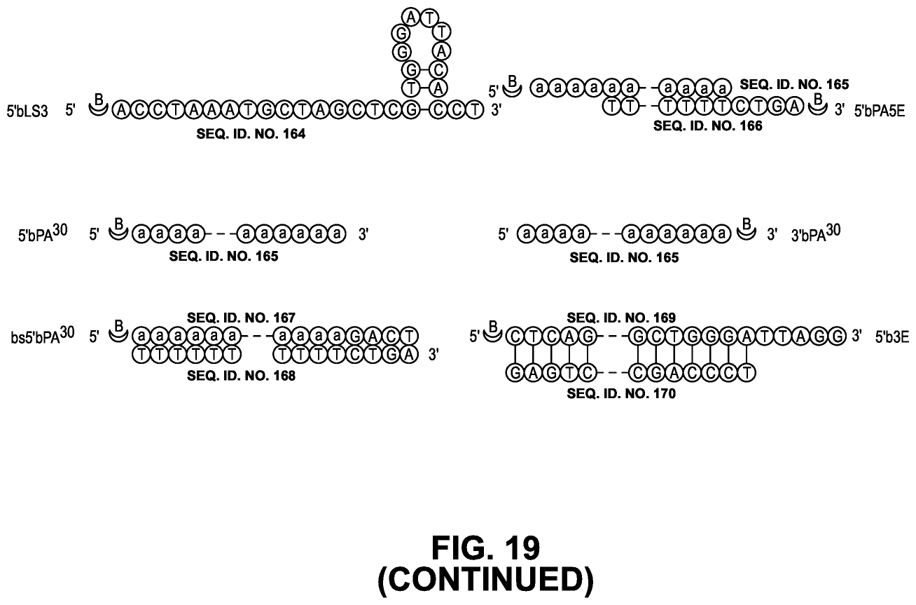

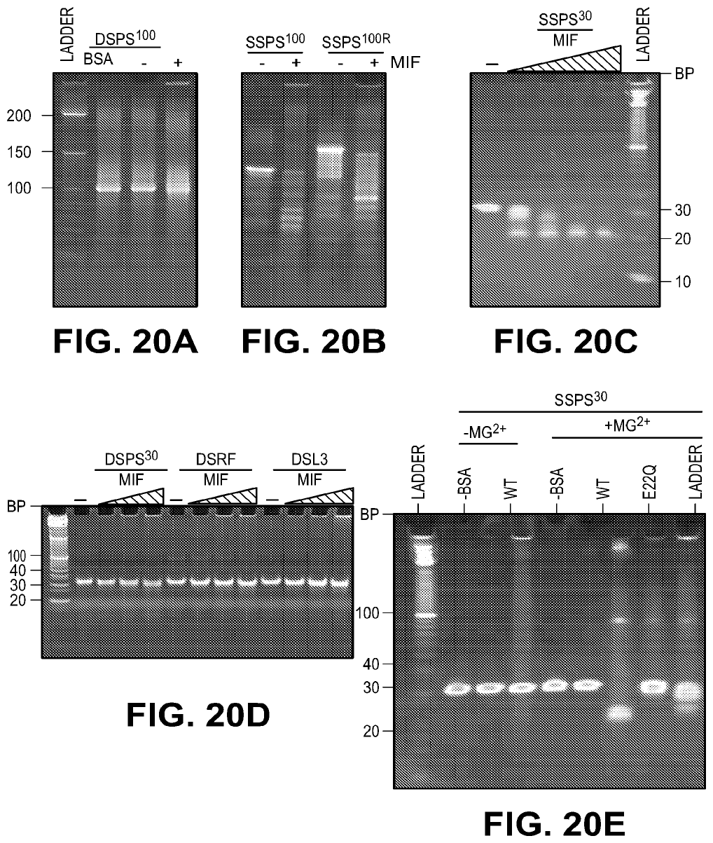

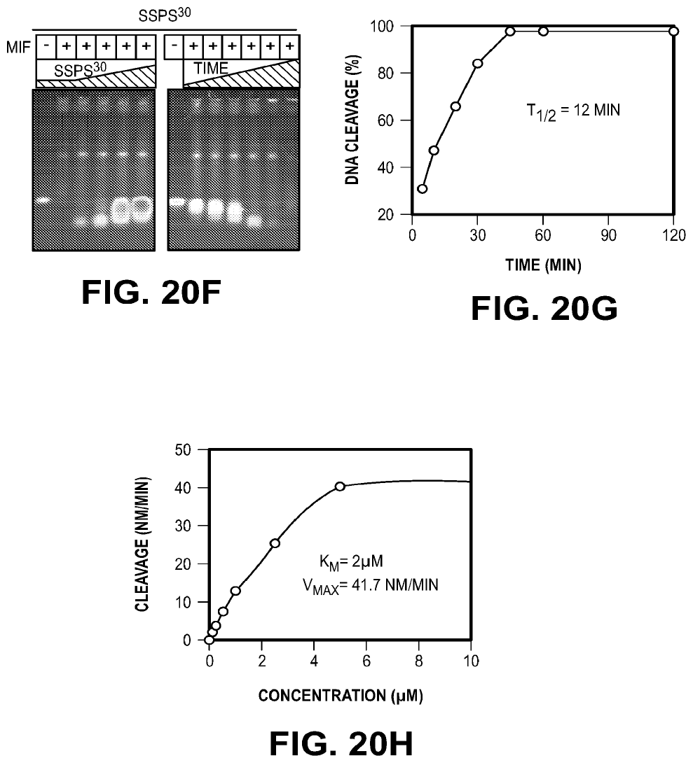

To determine whether MIF cleaves single or double stranded DNA, 35 random nucleotides were added to both the 5′ and 3′ ends of the PS³⁰ DNA binding motif and was designated PS¹⁰⁰ and cleavage of ssDNA (ssPS⁰⁰) or dsDNA (dsPS¹⁰⁰) was monitored. MIF substantially cleaves ssPS¹⁰⁰ and its complementary strand ssPS¹⁰⁰R, but not the dsPS¹⁰⁰ (FIGS. 20, A and B). The MIF DNA binding motif identified from the ChTP Seq (PS³⁰) is sufficient for MIF cleavage since increasing concentrations of MIF cleave ssPS³⁰ (FIG. 20C). However, increasing concentrations (1-4 μM) of MIF fail to cleave dsPS³⁰, its related sequence dsRF as well as its non-related sequence dsL3 (FIG. 20C). MIF cleavage of ssPS³⁰ requires Mg²⁺ (FIG. 20E). MIF E22Q and E22A mutations block the cleavage of ssPS³⁰ (FIG. 20220E). MIF cleaves ssPS³⁰ in a time dependent manner with a t_{1/2} of 12 minutes, and it cleaves ssPS³⁰ in a concentration dependent manner with a Kₘ of 2 μM and a Vₘₐₓ of 41.7 nM/min (FIG. 20, F to H). These kinetic properties are similar to other PD-D/E(X)K nucleases such as EcoRI (26, 31). MIF's preference for single stranded DNA is consistent with the 3-dimensional model of single stranded DNA binding to MIF's active site (FIG. 18B) and the MIF-DNA binding assays (FIG. 3B).

To determine whether MIF has sequence or structure specific endonuclease or exonuclease activity, a series of 5′ and 3′ biotin labeled variants based on the secondary structure of the DNA substrate ssPS³⁰ were synthesized, and MIF cleavage was examined (FIG. 3C and FIG. 19). It was found that MIF has 3′ exonuclease activity and it prefers to recognize and degrade unpaired bases at the 3′ end of ssPS³⁰, which is blocked by the biotin modification at the 3′ end (FIG. 3C lane 2-5 and FIG. 19, Table 1). MIF's 3′ exonuclease activity is also supported by the cleavage assays using the 5′bRF substrate, as well as 5′b3E substrate (FIG. 3C and FIG. 19, Table 1). Moreover, MIF's 3′ exonuclease activity allows it to cleave 5′ biotin-poly A (5′bPA³⁰), but not 3′ biotin-poly A (3′bPA³⁰), suggesting MIF's 3′ exonuclease activity is independent of the secondary structure (FIG. 3C and FIG. 19). MIF also possesses structurally specific endonuclease activity. It cleaves short unpaired bases of ssDNA at the 3′ end adjacent to the stem loop (5′bPS⁴⁰, 3′bPS⁴⁰, 5′b3F1, 3′b3F1 and 5′bL3) as well as 3′-OH/3′-biotin at the 3′ end adjacent to the stem loop (3′bSL and 3′bLF) (FIG. 3C and FIG. 19). In contrast to its exonuclease activity, MIF's endonuclease activity cannot be blocked by the biotin modification at the 3′ end (3′bSL, 3′bLF, 3′bPS⁴⁰ and 3′b3F1). 5′bL3 is a non-related PS³⁰ sequence, but with a similar stem loop structure that is cleaved by MIF, but with less efficiency (FIG. 3C and FIG. 19). Taken together these results indicate that MIF has both 3′ exonuclease and endonuclease activities and cleaves unpaired bases of stem loop ssDNA at the 3′ end.

To further study where MIF cleaves DNA and avoid the potential interference of biotin labeling, non-labeled PS³⁰ and 3F1 that only has 1 unpaired base at the 3′ end of the stem loop structure were used as substrates and two different DNA ladders based on PS³⁰ were customized. By incubating MIF (2 μM) with PS³⁰ for 2 h, two major products of 20 and 22 nucleotides are detected (FIG. 3D). In addition, faint higher molecular weight bands are also observed. These higher molecular weight bands are more obvious in the biotin labeled PS³⁰ MIF cleavage experiment where the incubation time was 1 h (FIG. 3D). MIF cleavage of the 3F1 substrate, only yields a 29 nt-band consistent with cleavage of 1 unpaired base at the 3′ end of the stem loop structure (FIGS. 3, D and E). These data suggest that PS³⁰ is initially cleaved by MIF after “A₂₃↓T₂₄↓T₂₅↓” using both 3′ exonuclease and endonuclease activity (FIG. 3E left panel). Then the resulting product forms a more stable structure (FIG. 3E right panel) and MIF cleaves at the new unpaired bases at the 3′ end of the stem loop structure after “G₂₀↓G₂₁↓G₂₂ ↓”. Taken together, MIF cleaves unpaired bases at the 3′ end adjacent to the stem loop at +1˜+3 positions using both 3′ exonuclease and endonuclease activities.

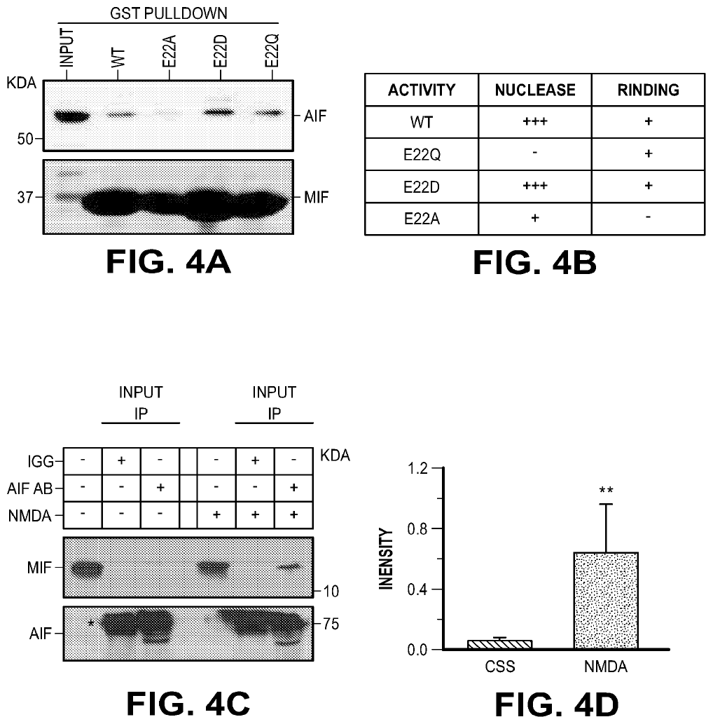

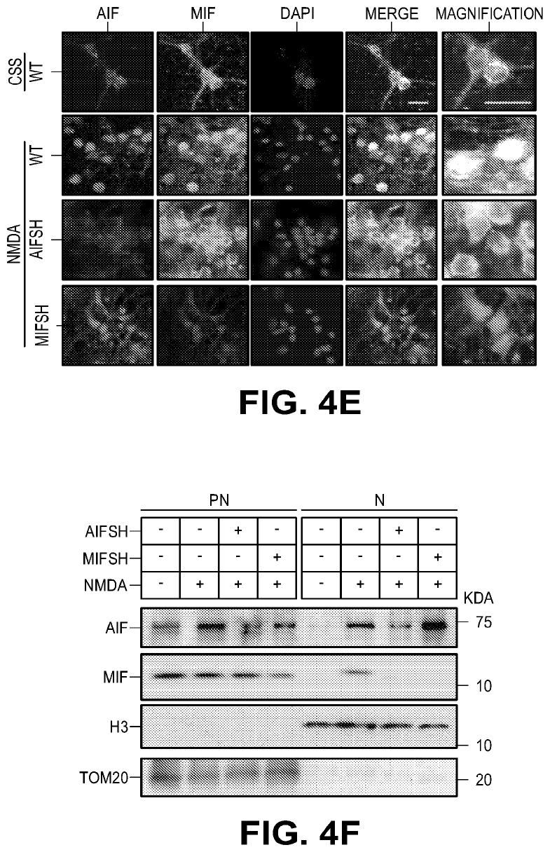

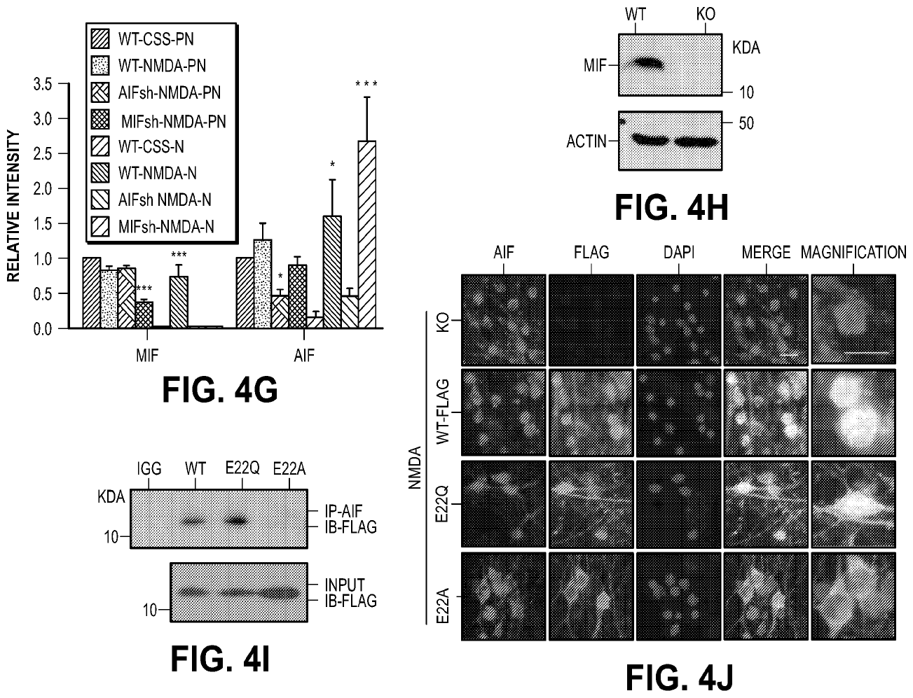

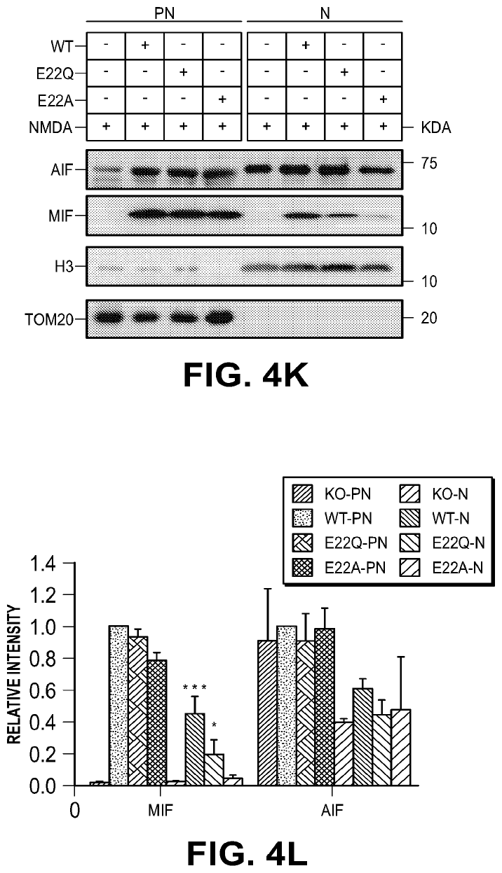

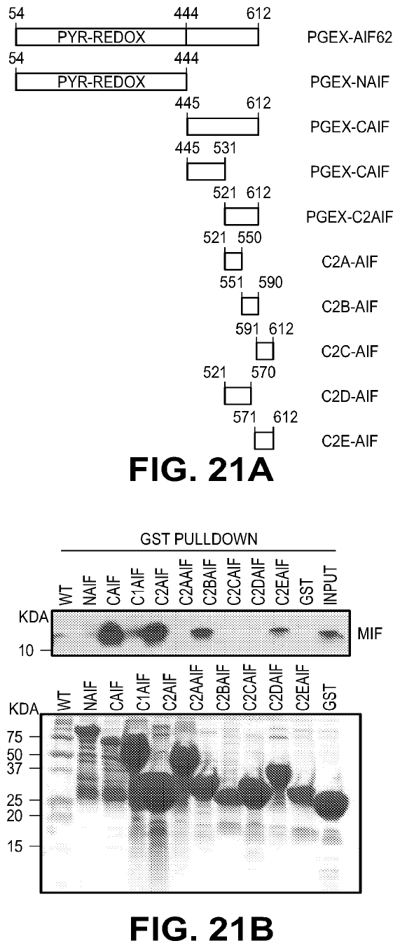

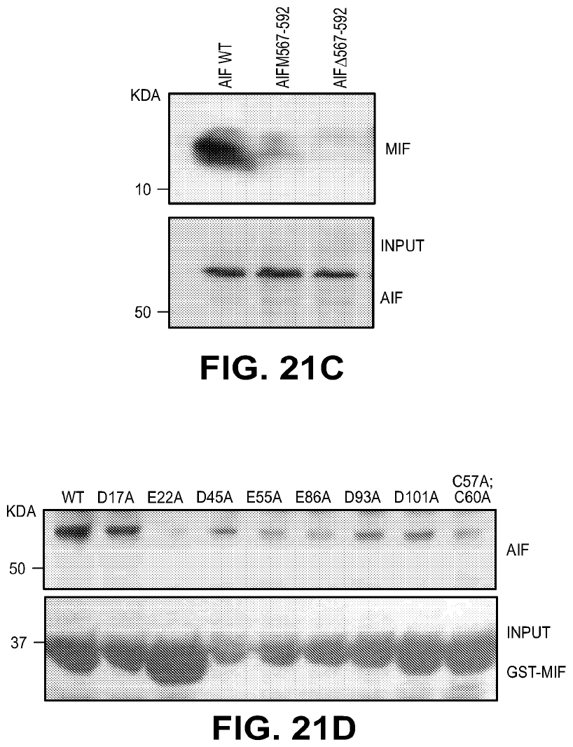

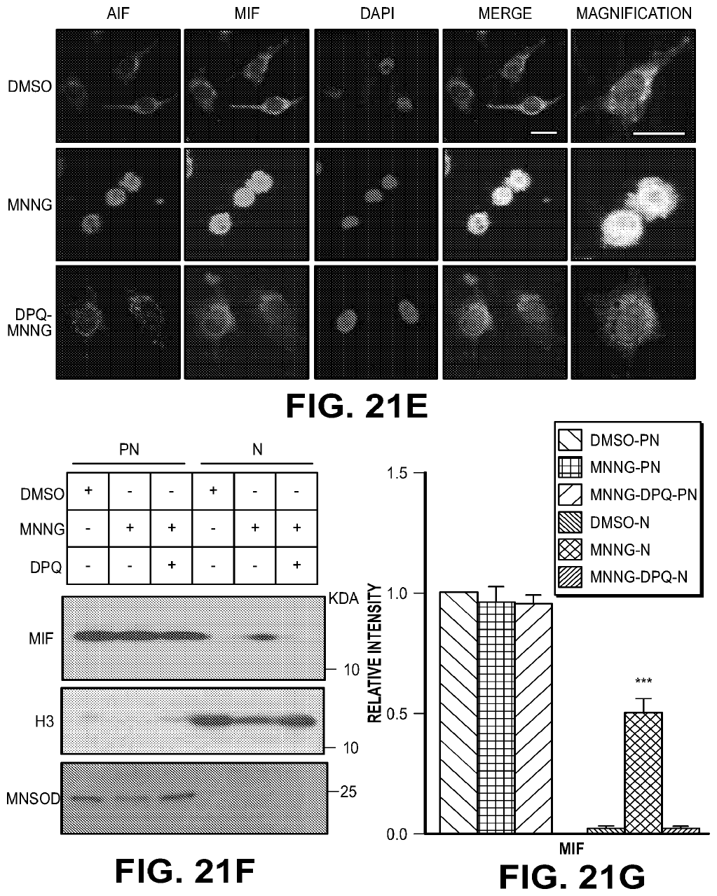

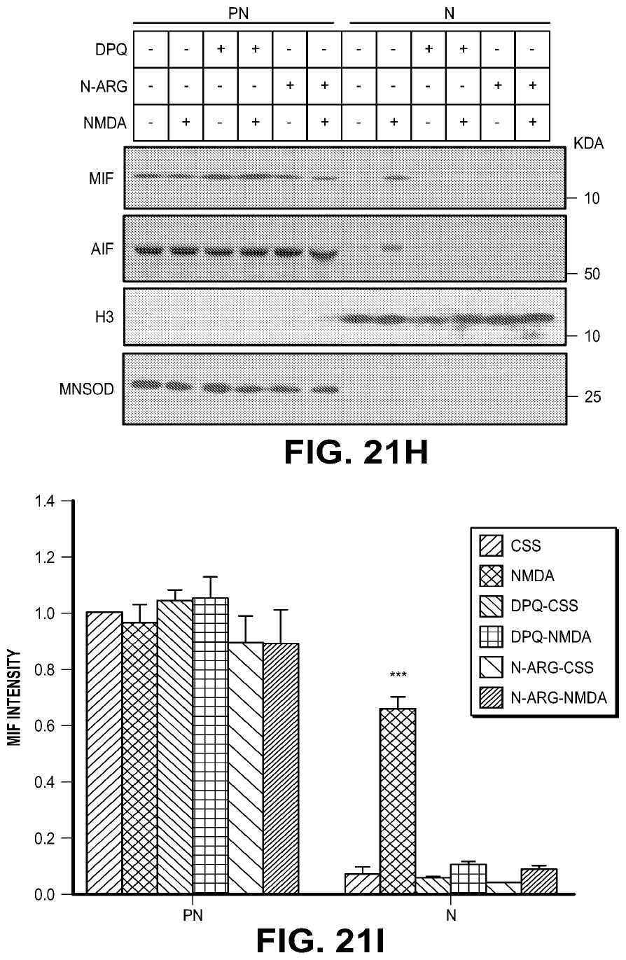

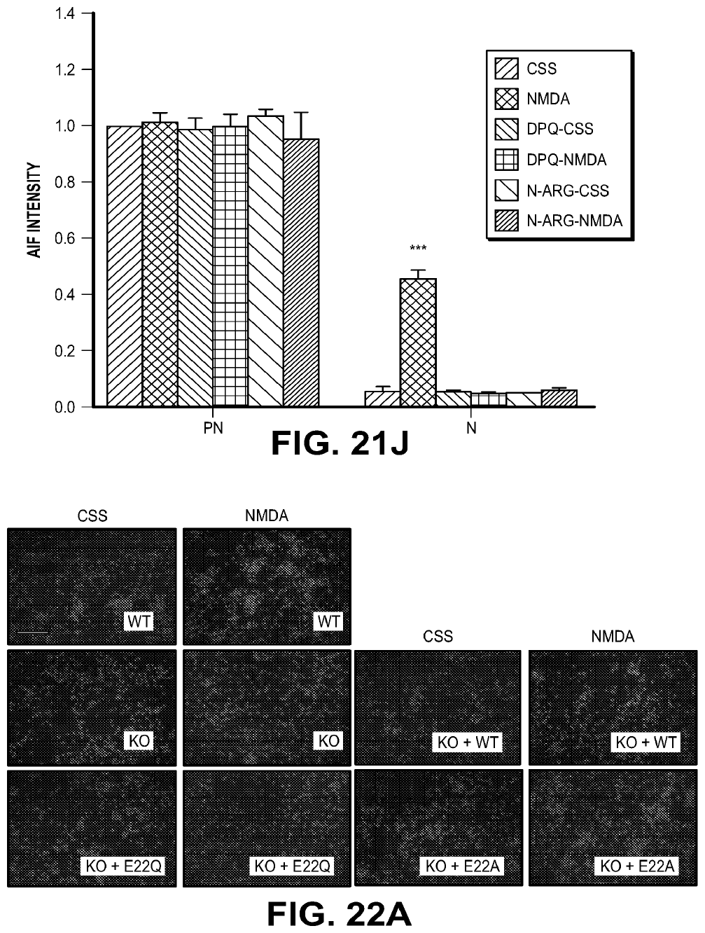

To confirm that MIF is an AIF interacting protein, GST pull down experiments were performed. Wild type GST-AIF pulls down endogenous MIF and wild type GST-MIF pulls down endogenous AIF (FIG. 4A and FIG. 21, A to D). Then the MIF-AIF binding domain was mapped. It was found that MIF binds to AIF at aa 567-592 (FIG. 21, A to C). Conversely, MIF E22A mutant has substantially reduced binding to AIF in the GST pull down, whereas the E22D and E22Q still bind to AIF (FIGS. 4, A and B, and FIG. 21D). In addition, the other PD-D/E(X)K and C57A; C60A mutations still bind to AIF (FIG. 21D). These data suggest that MIF E22 is critical for AIF binding. In line with GST pull down data, AIF co-immunoprecipitates MIF in cortical neurons treated with 500 μM NMDA, but is barely detectable in untreated cultures (FIGS. 4, C and D).

MIF is localized predominantly to the cytosol of both HeLa cells (FIG. 21E) and cortical neurons (FIG. 4E). Both MIF and AIF translocate to the nucleus and are co-localized within the nucleus upon stimulation by MNNG in HeLa cells and NMDA in cortical neurons. Knockdown of AIF leads to a loss of MIF translocation to the nucleus, but knockdown of MIF does not prevent translocation of AIF to the nucleus following NMDA exposure (FIG. 4E). Subcellular fractionation into nuclear and post-nuclear fractions confirms the translocation of MIF and AIF to the nucleus following NMDA exposure of cortical neuronal cultures and that AIF is required for MIF translocation (FIGS. 4, F and G). DPQ prevents accumulation of both MIF and AIF in the nucleus following NMDA administration in cortical neurons and MNNG treatment in HeLa cells (FIG. 21, E to J). Consistent with the notion that NMDA excitotoxicity involves nitric oxide production the nitric oxide synthase inhibitor, nitro-arginine (N-Arg), prevents accumulation of both MIF and AIF in the nucleus (FIG. 21H-J).

MIF is widely distributed throughout the brain and MIF knockout mice have previously been described (FIG. 4H) (32). Primary cortical cultures from MIF knockout mice were transduced with lentivirus carrying MIF-WT-FLAG, MIF-E22Q-FLAG, and MIF-E22A-FLAG to confirm the requirement of AIF/MIF binding for MIF nuclear accumulation following NMDA administration. Consistent with the GST pull down experiments (FIG. 4A), wild type MIF and E22Q interact with AIF but that MIF E22A does not bind to AIF (FIG. 4I). In non-transduced MIF knockout cultures and in MIF knockout cultures transduced with MIF-WT-FLAG, MIF-E22Q-FLAG, and MIF-E22A-FLAG, AIF translocates to the nucleus following NMDA administration (FIG. 4J). Both MIF wild type and MIF E22Q also translocate to the nucleus; however, AIF binding deficient mutant MIF E22A fails to do so (FIG. 4J). Subcellular fractionation into nuclear and post-nuclear fractions confirms the observations made by immunofluorescence (FIGS. 4, K and L). Taken together these results indicate that MIF's interaction with AIF is required for the nuclear translocation of MIF.

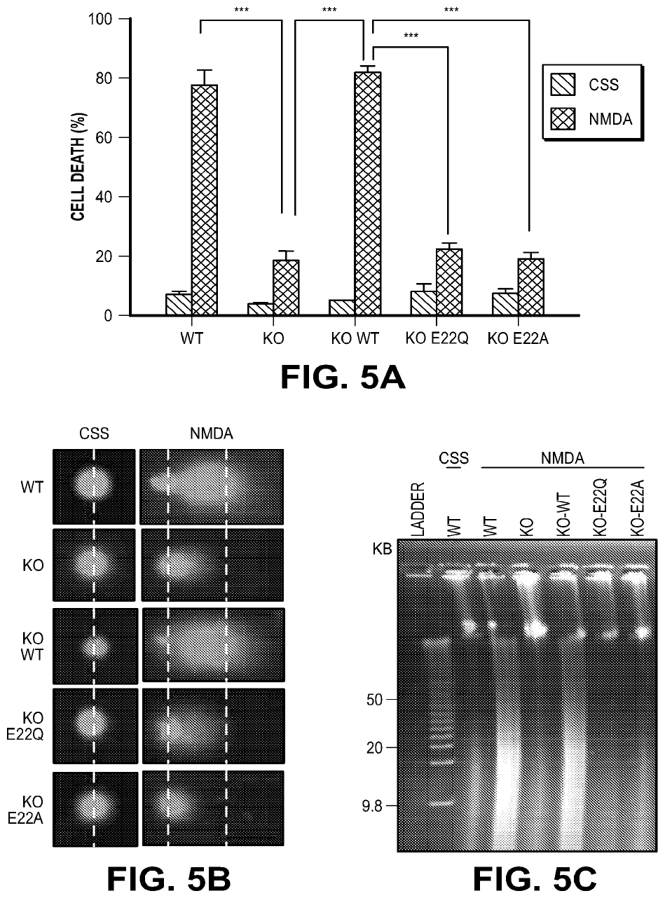

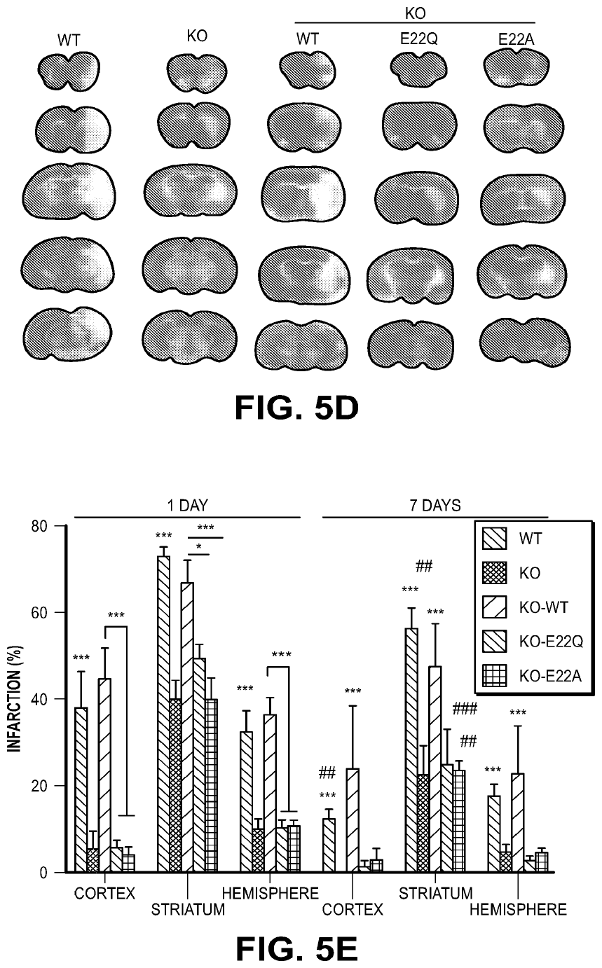

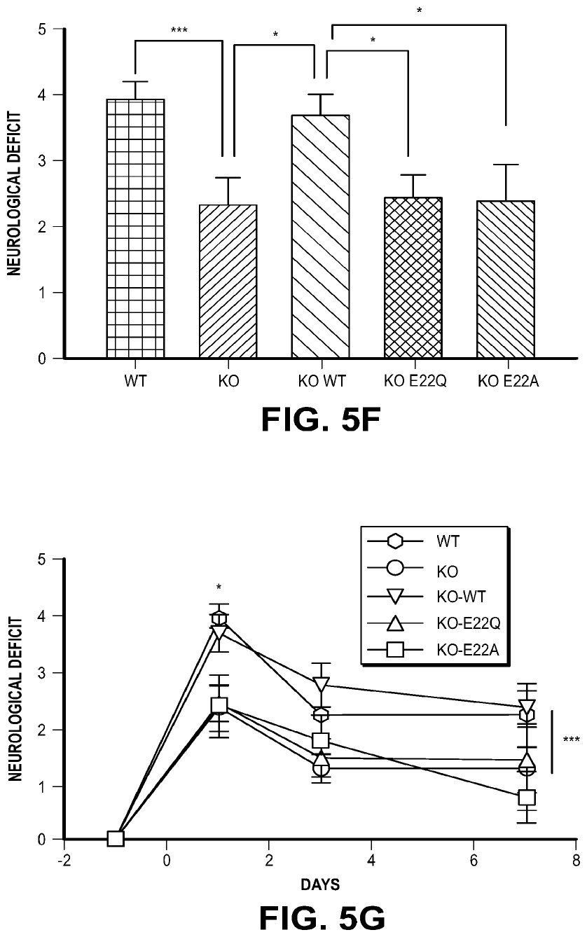

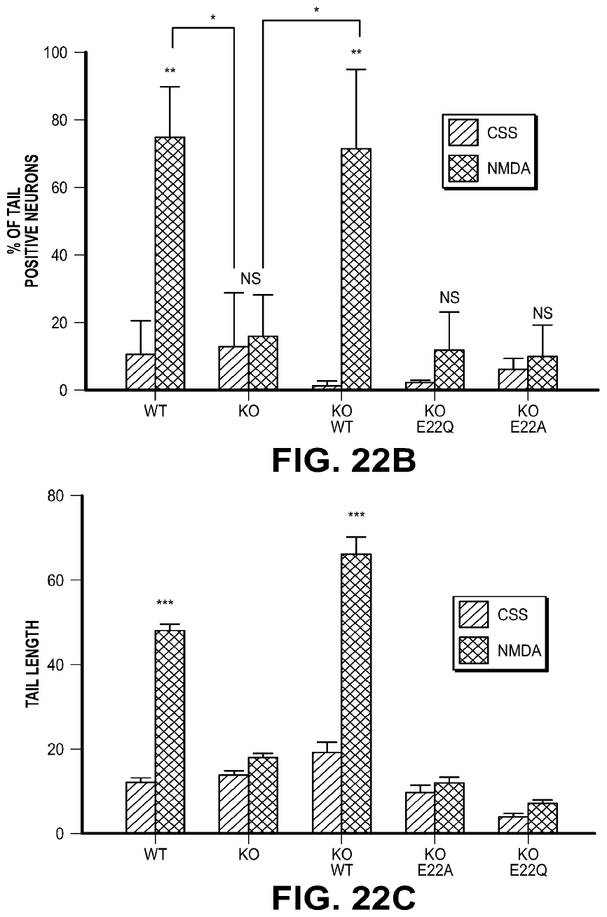

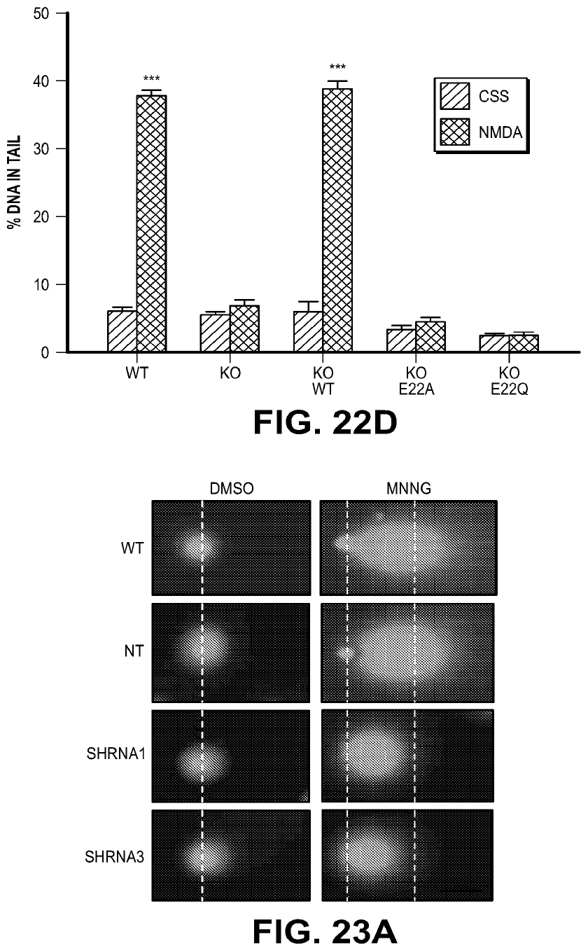

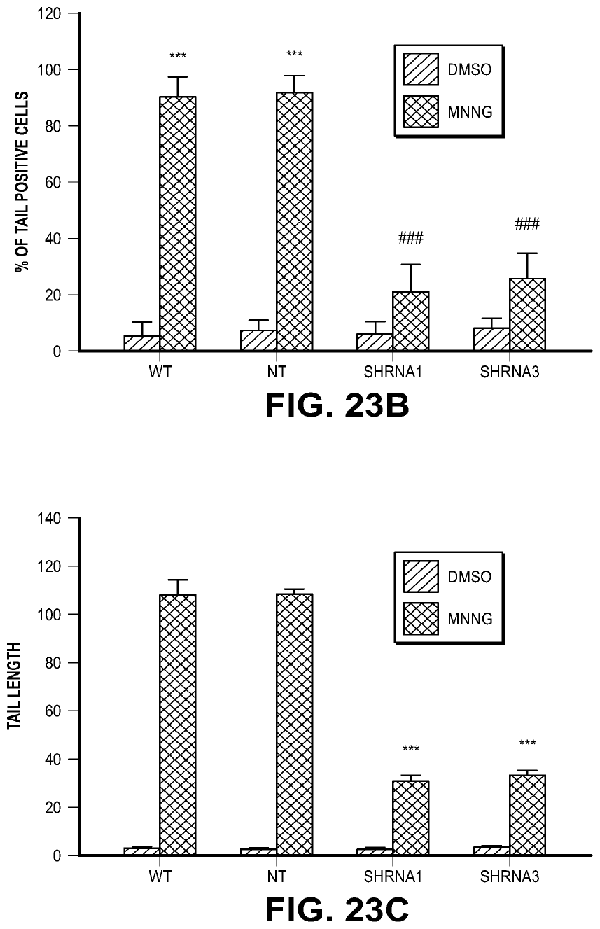

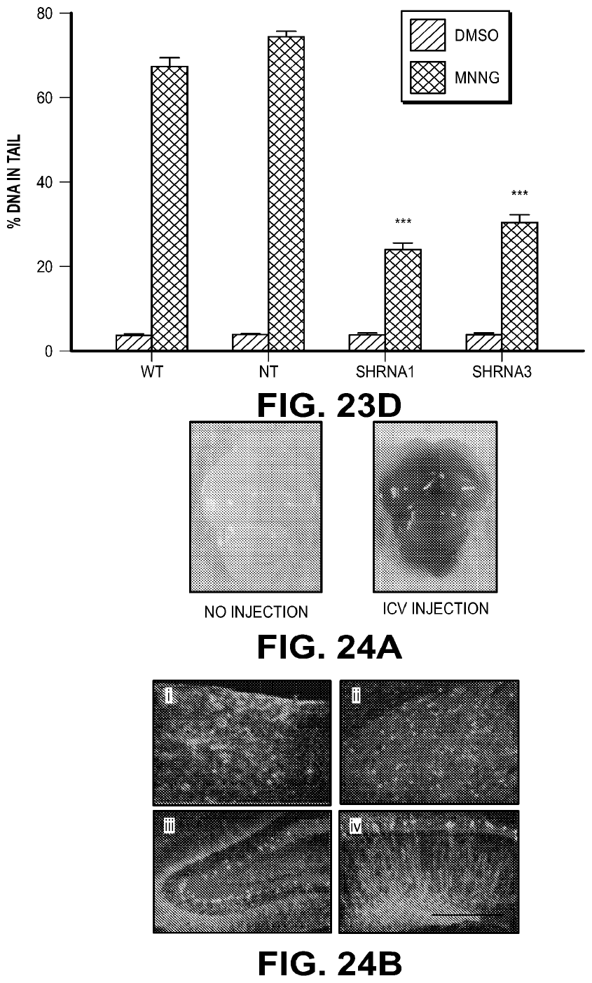

To determine if MIF's nuclease activity and AIF-mediated recruitment are required for parthanatos, MIF knockout cultures were transduced with the nuclease deficient MIF E22Q mutant and the AIF binding deficient mutant MIF E22A mutant. Consistent with the shRNA knockdown experiments, MIF knockout cortical cultures are resistant to NMDA excitotoxicity (FIG. 5A and FIG. 22A). Transduction with wild type MIF fully restores NMDA excitotoxicity, conversely, neither MIF E22Q nor MIF E22A restore NMDA excitotoxicity (FIG. 5A and FIG. 22A). By the comet assay, it was found that NMDA administration in wild type cortical neurons results in substantial numbers of neurons with comet tails, increased tail length and DNA in the tail, whereas MIF knockout neurons have no obvious comet tail positive neurons (FIG. 5B and FIG. 22, B to D). Transduction of knockout neurons with wild type MIF, but not with MIF E22Q or MIF E22A, restores comet tails, increases tail length and DNA in the tail following NMDA administration (FIG. 5B and FIG. 22, B to D). shRNA knockdown of MIF in HeLa cells with two different shRNAs results in a reduced number of cells with comet tails, reduced tail length and DNA in the tail as compared to non-targeted shRNA following MNNG administration (FIG. 23, A to D). A pulse field gel electrophoresis assay of genomic DNA confirms that NMDA administration causes large DNA fragments in wild type cortical neurons, but not in MIF knockout cortical neurons (FIG. 5C). No obvious large DNA fragments are observed in MIF knockout neurons transduced with MIF E22Q, or MIF E22A (FIG. 5C). Transduction of knockout neurons with wild type MIF restores NMDA-induced large DNA fragments (FIG. 5C). These results taken together indicate that MIF is the major nuclease involved in large scale DNA fragmentation due to MNNG or NMDA induced parthanatos.

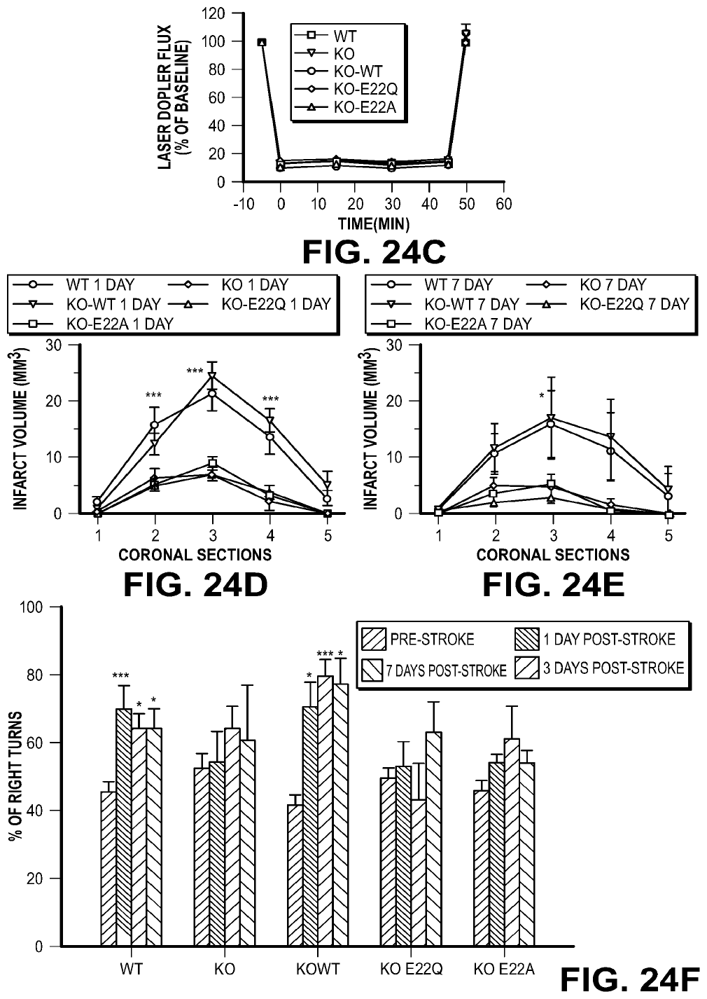

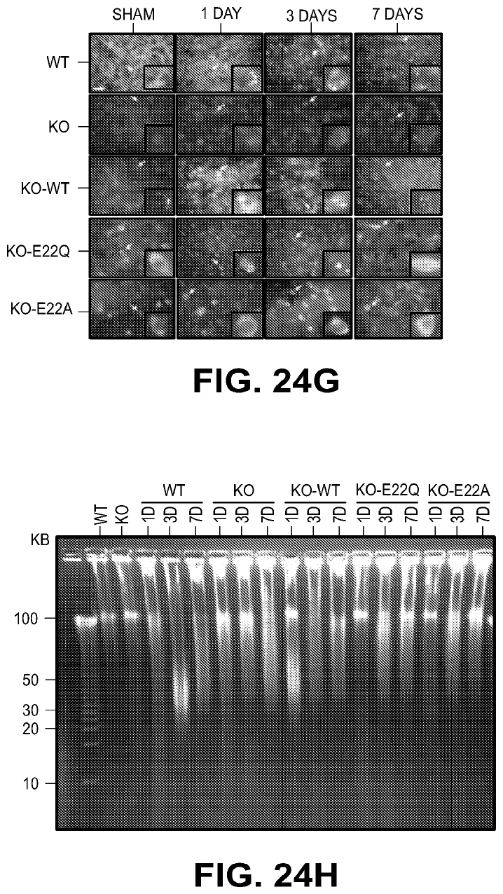

To evaluate the requirement of MIF nuclease activity and MIF binding to AIF in cell death due to parthanatos in vivo, MIF knockout mice were transduced with the nuclease deficient MIF E22Q mutant and the AIF binding deficient mutant MIF E22A mutant by injecting the intracerebroventricular zone of new born mice. Two-month old male mice were then subjected to 45-min transient occlusion of the middle cerebral artery (MCAO). The effectiveness of transduction was confirmed by immunostaining for MIF-FLAG in the cortex, striatum and hippocampus in adult mice (FIGS. 24, A and B). Despite the similar intensity of the ischemic insult (FIG. 24C), infarct volume is reduced in cortex, striatum and hemisphere by about 30% in MIF knockout mice compared to their wild-type counterparts (FIGS. 5, D and E, and FIGS. 24, D and E). Moreover, the neuroprotection in MIF knockout mice remains for at least 7 days (FIG. 5E and FIG. 24E). Expression of wild type MIF, but not MIF E22Q or MIF E22A, in the MIF knockout mice restores infarct volume to wild type levels (FIGS. 5, D and E, and FIGS. 24, D and E). Neurobehavior was assessed by spontaneous activity in the open field task at 1 day, 3 days and 7 days following MCAO. Consistent with the infarct data, MIF knockout mice have improved neurobehavioral scores compared to wild type. MIF knockout mice expressing wild type MIF have neurobehavioral scores equivalent to wild type mice while expression of MIF E22Q or MIF E22A are not significantly different from MIF knockout mice (FIGS. 5, F and G). Over 3 and 7 days the neurobehavioral scores of MIF knockout mice remain protected relative to wild type mice (FIGS. 5, F and G). Corner test data show that all mice do not show a side preference before MCAO surgery. However, wild type mice and MIF knockout mice expressing wild type MIF have significantly increased turning toward the non-impaired side at day 1, 3 and 7 after MCAO (FIG. 24F), indicating these mice have more severe sensory and motor deficits. No preference was observed in MIF knockout mice and MIF knockout mice with expression of MIF E22Q or MIF E22A (FIG. 24F). AIF and MIF localization was examined via confocal microscopy in the penumbra region of the stroke (FIG. 24G). Consistent with the observation in cortical neurons, AIF translocates to the nucleus at 1, 3 and 7 days after MCAO in MIF wild type, knockout as well as MIF knockout mice injected with MIF wild type, E22Q, and E22A (FIG. 24G). Both MIF wild type and MIF E22Q also translocate to the nucleus at 1, 3 and 7 days after MCAO; however, AIF binding deficient mutant MIF E22A fails to do so (FIG. 24G). DNA damage as assessed by pulse field gel electrophoresis is observed at day 1, 3 and 7 post MCAO with day 3 showing the most severe DNA damage in wild type mice or MIF KO mice expressing wild type MIF (FIG. 24H). DNA damage is reduced in the MIF KO mice and MIF KO mice expressing E22Q or E22A MIF (FIG. 24H). These data indicate that MIF is required for AIF mediated neurotoxicity and DNA cleavage and that AIF is required for MIF translocation in vivo.

A major finding of this invention is the identification of MIF as a PAAN. Using molecular modeling, it was shown that the MIF trimer contains the same topology structure as PD-D/E(X)K nuclease superfamily with a central four stranded mixed β-sheet next to two α-helices (24, 25). MIF has both 3′ exonuclease and endonuclease activity. It binds to 5′ unpaired bases of ssDNA with the stem loop structure and cleaves its 3′ unpaired bases. AIF interacts with MIF and recruits MIF to the nucleus where MIF binds and cleaves genomic DNA into large fragments similar to the size induced by stressors that activate parthanatos. Knockout of MIF markedly reduces DNA fragmentation induced by stimuli that activate PARP-1 dependent cell death. Mutating a key amino acid residue in the PD-D/E(X)K motif eliminates MIF's nuclease activity and protects cells from parthanatos both in vitro and in vivo. Disruption of the AIF and MIF protein-protein interaction prevents the translocation of MIF from the cytosol to the nucleus, which also protects against PARP-1 dependent cell death both in vitro and in vivo. Neither MIF's thiol-protein oxidoreductase activity or tautomerase activity is involved in its actions as a nuclease. Knockout of MIF, a MIF nuclease-deficient mutant and a MIF AIF binding deficient mutant all reduce infarct volume and have long lasting behavioral rescue in the focal ischemia model of stroke in mice. Thus, MIF is the long-sought after PAAN that is important in cell death due to activation of PARP-1 and the release of AIF (2).

Like PARP, inhibition of MIF nuclease activity is an attractive target for acute neurologic disorders. However, it may have advantages over PARP inhibition in chronic neurodegenerative diseases where PARP inhibition long term could impair the DNA damage response and repair. Inhibition of MIF's nuclease activity could bypass this potential concern and could offer an important therapeutic opportunity for a variety of disorders.

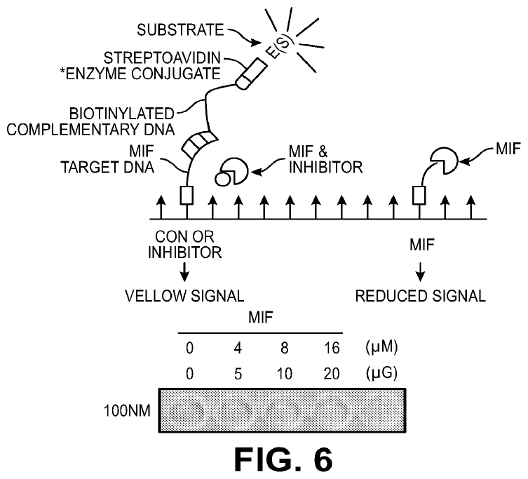

It was found that MIF has both 3′ exonuclease and endonuclease activity and its preferential DNA sequences for nuclease activity. This sequence is immobilized on DNA-BIND plates and incubated with recombinant MIF with or without pools from the macrocyclic compound library and hybridized with biotinylated complementary DNA. The sequence is detected by colorimetric changes measured by a spectrometer. If a pool contains a MIF inhibitor, the yellow substrate color will be maintained. If MIF is active, the DNA will be cleaved and the color will be lost (FIG. 6).

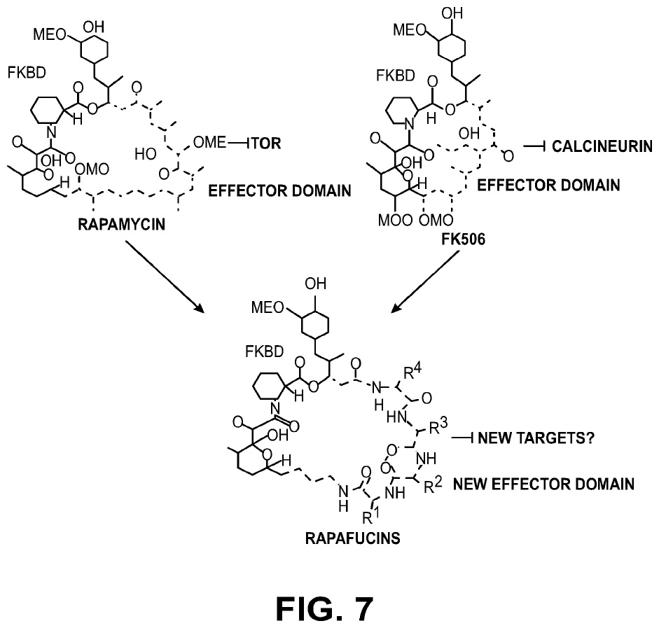

The macrocyclic natural products FK506 and rapamycin are approved immunosuppressive drugs with important biological activities. Structurally, FK506 and rapamycin share a similar FKBP-binding domain but differ in their effector domains. Switching the effector domain of FK506 and rapamycin can provide the changes of target from calcineurin to mTOR. Thus it is possible to functionally replace the effector domain to target proteins in the human proteome. A library of new macrocycles containing a synthetic FKBP-binding domain and a tetra-peptidyl effector domain, which are named rapafucins, were designed and generated to target new proteins (FIG. 7). Upon screening of the library, several hits that potently inhibit the nuclease activity of MIF have been identified.

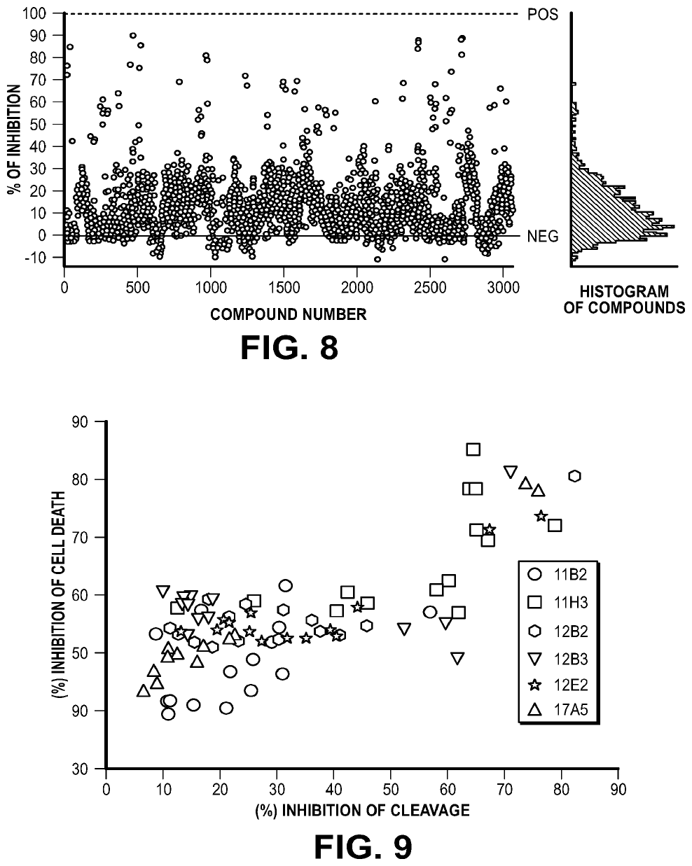

The hybrid macrocyclic library consists of 45,000 compounds in pools of 15 individual compounds. Thirty-eight plates (˜3000 pools) were screened and the screening of the pooled libraries was completed with the cleavage assay (FIG. 8). The compounds in the positive pools have tested individually in the cleavage assay and further assessed for neuroprotective actions in vitro in HeLa cells treated with MNNG as an inducing agent for parthanatos (FIG. 9). Twelve positive candidates were initially selected and tested in a dose-response DNA cleavage assay and MNNG-induced cell death assay, and then 4 candidates (C7; 12B3-11, C8; 12B3-11, C11; 17A5-1, C12; 17A5-2) were finally selected.

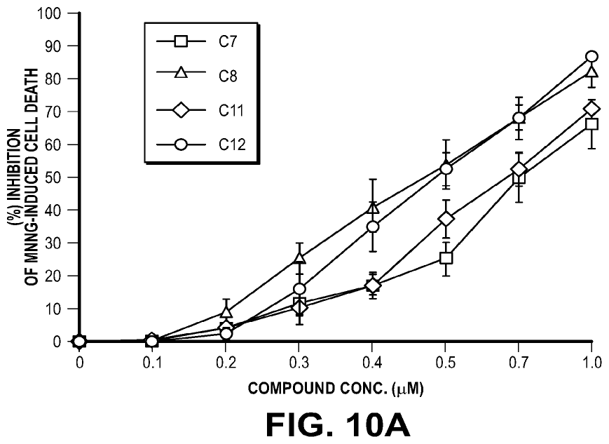

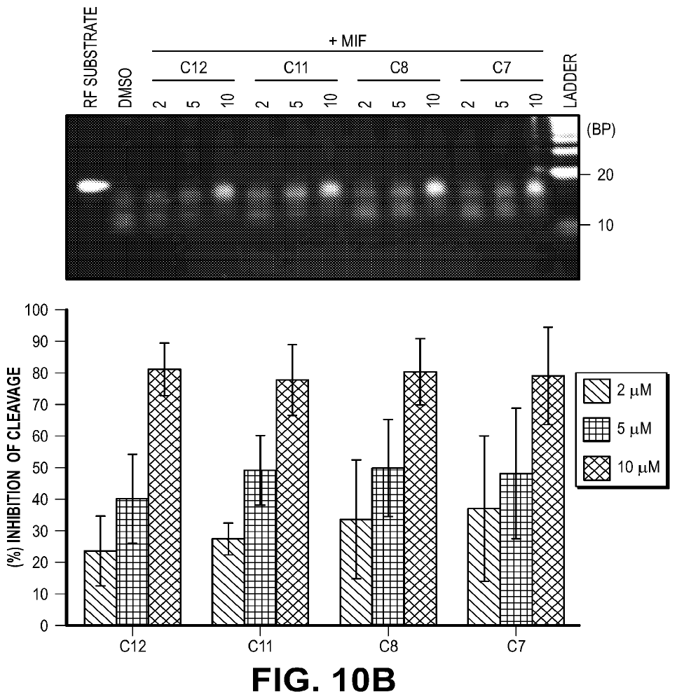

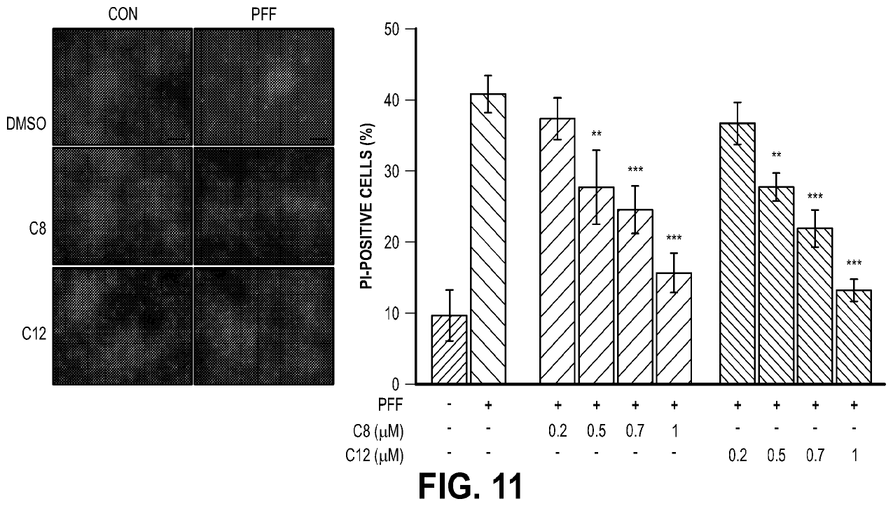

Positive candidates were advanced to a dose-response DNA cleavage assay in the TBE gel (FIG. 10A) and neuroprotective effects in HeLa cells treated with MNNG (FIG. 10B). Further, positive candidates were tested in α-synucelin pre-formed fibrils (α-Syn PFF) neurotoxicity. The treatment of recombinant misfolded α-syn PFF provides a model system of Parkinson's disease enabling the study of transmission and toxicity of α-synuclein in vitro and in vivo. Primary cortical cultures were exposed to PFF MIF inhibitors for 14 days. Cell viability was determined by computer assisted cell counting of Hoechst/propidium iodide positive cells. Here, C8 and C12 showed the most protective effect in PFF-induced toxicity, and the 2 hits were confirmed in a dose-response in PFF toxicity (FIG. 11).

Materials and Methods

Human Protein Chip High-Throughput Screening

16K and 5K human protein chips, which were prepared by spotting 16,000 or 5,000 highly purified proteins onto special nitrocellulose-coated slides (15), were incubated in renaturation buffer containing 50 mM Tris-HCl, pH 8.0, 100 mM NaCl, 1 mM DTT, 0.3% Tween 20 for 1 h at 4° C. After Blocking with 5% non-fat dry milk for 1 h at room temperature, protein chips were incubated with purified mouse AIF protein (50 nM, NP_036149) in 1% milk for 1 h. Protein interaction was then determined either by sequentially incubating with rabbit anti-AIF antibody (9, 11) and Alexa Fluor® 647 donkey anti-rabbit IgG, or Alexa Fluor® 647 donkey anti-rabbit IgG only as negative control. Protein microarrays were scanned with GenePix 4000B Microscanner (Tecan) using the Cy5 image and the median fluorescence of each spot was calculated. The same procedure described previously to identify interacting proteins was used (15).

Reverse Transfection Format siRNA-Based Screen for PARP-1-Dependent Cell Viability.

On-Target Plus™ SMARTpool® siRNAs targeting AIF-interacting proteins resulting from human protein chip high throughput screening were customized in 96-well plates from Dharmacon. The plates were rehydrated using DharmaFECT 1 transfection reagent at room temperature for 30 min. HeLa cells were then seeded in the plates with the cell density at 1×104/well. 48 h after transfection, cells were treated with MNNG (50 μM) or DMSO for 15 min and then incubated in normal complete medium for 24 h. After adding alamarBlue for 1-4 h, cell viability was determined by fluorescence at excitation wavelength 570 nm and emission wavelength 585 nm. PARP-1 siRNAs were used as the positive control and non-target siRNAs as the negative control.

Nuclease Assays

Human genomic DNA (200 ng/reaction, Promega), pcDNA (200 ng/reaction) or PS³⁰ and its related and non-related substrates (1 μM) was incubated with wild type MIF or its variants at a final concentration of 0.25-8 μM as indicated in 10 mM Tris-HCl buffer (pH 7.0) containing 10 mM MgCl₂ and 1 mM DTT or specific buffer as indicated, for 1 h (with pcDNA and small DNA substrates) or 4 h (with human genomic DNA) at 37° C. The reaction was terminated with loading buffer containing 10 mM EDTA and incubation on ice. The human genomic DNA samples were immediately separated on a 1.2% pulse field certified agarose in 0.5×TBE buffer with initial switch time of 1.5 s and a final switch time of 3.5 s for 12 h at 6 V/cm. pcDNA samples were determined by 1% agarose gel. Small DNA substrates were separated on 15% or 25% TBE-urea polyacrylamide (PAGE) gel or 20% TBE PAGE gel. Then gel was stained with 0.5 μg/ml Ethidium Bromide (EtBr) followed by electrophoretic transfer to nylon membrane. Then, Biotin-labeled DNA is further detected by chemiluminescence using Chemiluminescent Nucleic Acid Detection Module (Thermo Scientific).

Electrophoretic Mobility Shift Assay (EMSA)

EMSA assay was performed using LightShift Chemiluminescent EMSA kit (Thermo Scientific) following the manufactures instruction. Briefly, purified MIF protein (2 μM) was incubated with biotin-labeled DNA substrates (10 nM) in the binding buffer containing 10 mM MgCl₂ for 30 min on ice. Then samples were separated on 6% retardation polyacrylamide followed by electrophoretic transfer to nylon membrane. Then, Biotin-labeled DNA is further detected by chemiluminescence using Chemiluminescent Nucleic Acid Detection Module (Thermo Scientific).

Comet Assay

Comet assays were conducted following protocols provided by Trevigen (Gaithersburg, Md.). Briefly, HeLa cells with or without MNNG treatment and cortical neurons with or without NMDA treatment were washed with ice-cold PBS 6 h after the treatment, harvested by centrifugation at 720 g for 10 min and re-suspended in ice-cold PBS (Ca²⁺ and Mg²⁺ free) at 1×105 cells/ml. C ells were then combined with 1% low melting point agarose in PBS (42° C.) in a ratio of 1:10 (v/v), and 50 μl of the cell-agarose mixture was immediately pipetted onto the CometSlide and placed flatly at 4° C. in the dark for 30 min to enhance the attachment. After being lysed in lysis buffer, slides were immersed with alkaline unwinding solution (200 mM NaOH, pH>13, 1 mM EDTA) for 1 h at RT. The comet slides were transferred and electrophoresed with 1 L of alkaline unwinding solution at 21 Volts for 30 min in a horizontal electrophoresis apparatus. After draining the excess electrophoresis buffer, slides were rinsed twice with dH2O and then fixed with 70% ethanol for 5 min and stained with SYBR Green for 5 min at 4° C. Cell images were captured using a Zeiss epifluorescent microscope (Axiovert 200M) and image analysis was performed with a CASP software (version 1.2.2). The length of the “comet tail,” which is termed as the length from the edge of the nucleus to the end of the comet tail, for each sample, was measured.

Protein Expression and Purification

Human endoG (NM_004435), cyclophilin A (NM_021130), mouse AIF (NM_012019), human MIF (NM_002415) cDNA and their variants were subcloned into glutathione S-transferase (GST)-tagged pGex-6P-1 vector (GE Healthcare) by EcoRI and XhoI restriction sites and verified by sequencing. The protein was expressed and purified from Escherichia coli by glutathione Sepharose. The GST tag was subsequently proteolytically removed for the nuclease assay. MIF point mutants were constructed by polymerase chain reaction (PCR) and verified by sequencing. The purity of MIF proteins that were used in the nuclease assays was further confirmed by mass spectrometry. MIF proteins purified by FPLC were also used in the nuclease assays and no obvious difference was observed between FPLC MIF and non-FPLC MIF proteins. GST protein was used as a negative control in the nuclease assay.

Middle Cerebral Artery Occlusion (MCAO)

Cerebral ischemia was induced by 45 min of reversible MCAO as previously described (33). Adult male MIF KO mice (2-to-4 month-old, 20-28 g) were anesthetized with isoflurane and body temperature was maintained at 36.5±0.5° C. by a feedback-controlled heating system. A midline ventral neck incision was made, and unilateral MCAO was performed by inserting a 7.0 nylon monofilament into the right internal carotid artery 6-8 mm from the internal carotid/pterygopalatine artery bifurcation via an external carotid artery stump. Sham-operated animals were subjected to the same surgical procedure, but the suture was not advanced into the internal carotid artery. After 1 day, 3 days or 7 days of reperfusion, MIF WT and KO mice were perfused with PBS and stained with triphenyl tetrazolium chloride (TTC). The brains were further fixed with 4% PFA and sliced for the immunohistochemistry staining (9, 11, 15, 34).

ChIP-Seq

ChIP-seq was performed as previously described (35, 36). Briefly, HeLa Cells were first treated with DMSO or MNNG (50 μM, 15 min). 5 h after MNNG treatment, cells were cross-linked with 1% formaldehyde for 20 min at 37° C., and quenched in 0.125 M glycine. Chromatin extraction was performed before sonication. The anti-MIF antibody (ab36146, Abcam) was used and DNA was immunoprecipitated from the sonicated cell lysates. The libraries were prepared according to Illumina's instructions accompanying the DNA Sample kit and sequenced using an Illumina HiSeq2000 with generation of 50 bp single-end reads.

Detailed procedures are as follows. HeLa cells were treated with DMSO or MNNG (50 μM) for 15 min and cultured in the fresh medium for additional 5 h. Cells then were cross-linked with 1% formaldehyde for 10 min at 37° C., and the reaction was quenched in 0.125 M glycine for 20 min at room temperature. Chromatin was extracted using SimpleChIP® Enzymatic Chromatin IP kit from Cell Signaling Technology (Cat #9003), and sonicated 30 sec on and 30 sec off for 15 cycles using Bioruptor Twin (Diagenode). The quality and size of sheared chromatin DNA were examined on an agarose gel by DNA electrophoresis. 10% of chromatin was kept as input and the rest of the chromatin was diluted and pre-cleared using 10 μl Magnetic protein G agarose slurry for 30 minutes at 4° C. to exclude nonspecific binding to protein G agarose beads directly. The pre-cleared chromatin was incubated overnight with an anti-MIF antibody (3 μg/ml, ab36146, Abcam) or control IgG (3 μg/ml) in the presence of Magnetic protein G agarose slurry (30 μl) at 4° C. After washing the protein G agarose beads for 3 times, half of the protein G agarose/antibody complex was subjected to immunoblot assays to check the quality of the immunoprecipitation. Another half of the protein G agarose/antibody complex was eluted in 170 μl of elution buffer containing 1% SDS, 0.1M NaHCO₃ at 65° C. The eluates as well as the chromatin input were treated with 1 mg/ml RNase A at 37° C. for 30 min, and reverse-crosslinked by incubating at 65o C for 4 h after adding 3 μl of 5 M NaCl and 1 μl of 10 mg/ml proteinase K. Finally the chromatin DNA was purified using phenol/chloroform/isoamyl alcohol and precipitated by ethanol. The ChTP and input DNA libraries were prepared using Illumina's Truseq DNA LT Sample Prep Kit according to the instructions. The final product was amplified for 15 cycles. The quality and the size of the insert was analyzed using a bioanalyzer. Sequencing was performed in the Next Generation Sequencing Center at Johns Hopkins using an Illumina HiSeq2000 with generation of 50 bp single-end reads. The ChIP-seq raw data have been deposited in the GEO database accession #: GSE65110.

ChIP-Seq Data Analysis

Raw data from the HiSeq2000 was converted to FASTQ using CASAVA v1.8 and demultiplexed. Reads were mapped to the human genome (hgl9) using Bowtie2 (v2.0.5) using the default parameters. Converted SAM files were passed to MACS (v1.4.1) for peak calling using the default parameters. Peaks from DMSO- and MNNG-treated libraries were reported in bed format and are provided in GEO. Peaks differentially identified in the DMSO- and MNNG-treated groups were parsed by a custom R script. Sequence corresponding to peaks identified in only MNNG-treated, but not DMSO-treated libraries were fed into SeSiMCMC_4_36, Chipmunk v4.3+, and MEMEchip v4.9.0 for motif discovery using default parameters.

Data transfer: The CASAVAv1.8 software was used to convert the raw files into fastq files as well deMultiplex the lanes.

### DMSO_MIF: JHUTD01001/JHUTD01001_001_DPAN1/raw

### DMSO_Input: JHUTD01001/JHUTD01001_002_Dinput1/raw

### MNNG_MIF: JHUTD01001/JHUTD01001_003_MPAN1/raw

### MNNG_Input: JHUTD01001/JHUTD01001_004_Minput1/raw

Analysis: The following is a list of analysis steps along with the parameters used by that step. All the motif finding software was run using default settings.

1. Alignment Pipeline

a. Bowtie2-2.0.5 with default parameters to perform fragment alignments to the hg19 genome, generating a single SAM file

### JHUTD01001/JHUTD01001_001_DPAN1/DPan1_hg19_alignment.sam

### JHUTD01001/JHUTD01001_002_Dinput1/Dinput1_hg19_alignment.sam

### JHUTD01001/JHUTD01001_003_MPAN1/MPan1_hg19_alignment.sam

### JHUTD01001/JHUTD01001_004_Minput1/Minput1_hg19_alignment.sam

b. Sort and convert SAM file to BAM file using samtools-0.1.18/

### JHUTD01001/JHUTD01001_001_DPAN1/DPan1_hg19_alignment.bam

### JHUTD01001/JHUTD01001_002_Dinput1/Dinput1_hg19_alignment.bam

### JHUTD01001/JHUTD01001_003_MPAN1/MPan1_hg19_alignment.bam

### JHUTD01001/JHUTD01001_004_Minput1/Minput1_hg19_alignment.bam

2. Peak Calls Using MACS-1.4.1 Using Default Parameters

a. Peak Call

### JHUTD01001/JHUTD01001_000_analysis/MACS/DPan1_vs_Dinput_peaks.bed

### JHUTD01001/JHUTD01001_000_analysis/MACS/MPan1_vs_Minput_peaks.bed

b. Annotated Peak Calls

### JHUTD01001/JHUTD01001_000_analysis/MACS/DPan1_vs_Dinput_annotation.txt

### JHUTD01001/JHUTD01001_000_analysis/MACS/MPan1_vs_Minput_annotation.txt

c. Custom Rscript to Perform Differential Peak Calls Based on Genes

JHUTD01001/JHUTD01001_000_analysis/MACS/intersections.bothsamples.DPan1 MPan1.txt

### JHUTD01001/JHUTD01001_000_analysis/MACS/intersectionsDPan1_not_MPan1.txt

### JHUTD01001/JHUTD01001_000_analysis/MACS/intersectionsMPan1_not_DPan1.txt

d. Annotated Differential Peak Calls

### JHUTD01001/JHUTD01001_000_analysis/MACS/only_DPan1_annotation.txt

### JHUTD01001/JHUTD01001_000_analysis/MACS/only_MPan1_annotation.txt

3. Coverage Tracks to View Alignments Created Through IGVtools

### JHUTD01001/JHUTD01001_000_analysis/coverage_analysis/DPan1.tdf

### JHUTD01001/JHUTD01001_000_analysis/coverage_analysis/Dinput1.tdf

### JHUTD01001/JHUTD01001_000_analysis/coverage_analysis/MPan1.tdf

### JHUTD01001/JHUTD01001_000_analysis/coverage_analysis/Minput1.tdf

4. Motifs were Found Using Three Different Software

a. SeSiMCMC_4_36

### JHUTD01001/JHUTD01001_000_analysis/motif/SeSiMCMC_motif_Dpan1.txt

### JHUTD01001/JHUTD01001_000_analysis/motif/SeSiMCMC_DPan1_logo.png

### JHUTD01001/JHUTD01001_000_analysis/motif/SeSiMCMC_MPan1_motif.txt

### JHUTD01001/JHUTD01001_000_analysis/motif/SeSiMCMC_MPan1_logo.pdf

b. Chipmunk v4.3+

### JHUTD01001/JHUTD01001_000_analysis/motif/DPan1_ChiPMunk_motif.txt

### JHUTD01001/JHUTD1001_000_analysis/motif/MPan1_ChipMunk_motif.txt

c. MEMEchip v4.9.0

JHUTD01001/JHUTD01001_000_analysis/motif/MEME ChIP_DPan1.webarchive
JHUTD01001/JHUTD01001_000_analysis/motif/MEME ChIP_MPan1.webarchive
JHUTD01001/JHUTD01001_000_analysis/motif/only_MPan1_MEME_ChTP.webarchive

5. CEAS Software to Generate Plots for Region Annotation, Gene Centered Annotation and Average Signal Profiling Near Genomic Features

### JHUTD01001/JHUTD01001_000_analysis/CEAS/DPan1.pdf

### JHUTD01001/JHUTD01001_000_analysis/CEAS/MPan1.pdf

### JHUTD01001/JHUTD01001_000_analysis/CEAS/MPan1_only.pdf

MIF-DNA Docking Methods

A DNA duplex structure (37) (PDB accession 1BNA) and a single-stranded DNA structure (PDB accession 2RPD (38)) were docked onto the surface of MIF (PDB accession 1FIM (23)) using Hex-8.0. protein-DNA docking program (39, 40). The Hex program uses a surface complementarity algorithm to identify contact between protein and DNA. MIF surfaces were generated using Pymol. All images were viewed and labeled with pdb viewer, Pymol. The MIF-DNA docked models are shown as obtained from the HEX program.

Lentivirus, Adeno-Associated Virus (AAV) Construction and Virus Production

Mouse MIF-WT-Flag (NM_010798), MIF-E22Q-Flag and MIF-E22A-Flag were subcloned into a lentiviral cFugw vector by AgeI and EcoRI restriction sites, and its expression was driven by the human ubiquitin C (hUBC) promoter. Human MIF and mouse MIF shRNAs were designed using the website katahdin.cshl.org/siRNA/RNAi.cgi?type=shRNA. The program gave 97 nt oligo sequences for generating shRNAmirs. Using PacI SME2 forward primer 5′ CAGAAGGTTAATTAAAAGGTATATTGCTGTTGACAGTGAGCG 3′ SEQ ID NO: 1 and NheI SME2 reverse primer 5′ CTAAAGTAGCCCCTTGCTAGCCGAGGCAGTAGGCA 3′ SEQ ID NO: 2. PCR was performed to generate the second strand, and PacI and NheI restriction sites were added to clone the products into pSME2, a construct that inserts an empty shRNAmir expression cassette in the pSM2 vector with modified restriction sites into the cFUGw backbone. This vector expresses GFP. The lentivirus was produced by transient transfection of the recombinant cFugw vector into 293FT cells together with three packaging vectors: pLP1, pLP2, and pVSV-G (1.3:1.5:1:1.5). The viral supernatants were collected at 48 and 72 hours after transfection and concentrated by ultracentrifuge for 2 hours at 50,000 g.

MIF-WT-Flag, MIF-E22Q-Flag and MIF-E22A-Flag were subcloned into a AAV-WPRE-bGH (044 AM/CBA-pI-WPRE-bGH) vector by BamHI and EcoRI restriction sites, and its expression was driven by chicken β-actin (CBA) promoter. All AAV2 viruses were produced by the Vector BioLabs.

Sequences of MIF Substrates, Templates and Primers

Sequences of MIF substrates, templates and primers used for shRNA constructs and point mutation constructs are as follows.

PS¹⁰⁰-
SEQ ID NO: 3
5′ACCTAAATGCTAGAGCTCGCTGATCAGCCTCGACTCTCAGCCTCCCAAGTAGCTGG
GATTACAGGTAAACTTGGTCTGACAGTTACCAATGCTTAATGAG3′,
PS^{100R}-
SEQ ID NO: 4
5′CTCATTAAGCATTGGTAACTGTCAGACCAAGTTTACCTGTAATCCCAGCTACTTGG
GAGGCTGAGAGTCGAGGCTGATCAGCGAGCTCTAGCATTTAGGT3′,
PR³⁰
SEQ ID NO: 5
5′CTCAGCCTCCCAAGTAGCTGGGATTACAGG3′,
SL-
SEQ ID NO: 6
5′CCTGTAATCCCAAGTAGCTGGGATTACAGG3′,
LF-
SEQ ID NO: 7
5′AAAAAAACTCAGCCTCCCAAGTAGCTGGGA3′,
RF-
SEQ ID NO: 8
5′TCCCAAGTAGCTGGGATTACAGGAAAAAAA3′,
PA³⁰-
SEQ ID NO: 9
5′AAAAAAAAAAAAAAAAAAAAAAAAAAAAAA3′,
3E-
SEQ ID NO: 5
5′CTCAGCCTCCCAAGTAGCTGGGATTACAGG3′,
SEQ ID NO: 10
5′TCCCAGCTACTTGGGAGGCTGAG3′,
PS⁴⁰-
SEQ ID NO: 11
5′CTCAGCCTCCCAAGTAGCTGGGATTACAGGTAAACTTGGT3′,
3F1
SEQ ID NO: 12
5′AAAAAAAAAACAAGTAGCTGGGATTACAGG3′,
L3-
SEQ ID NO: 13
5′ACCTAAATGCTAGAGCTCGCTGATCAGCCT3′;
hMIFshRNA1-
SEQ ID NO: 14
TGCTGTTGACAGTGAGCGCTCATCGTAAACACCAACGTGCTAGTGAAGCCACAGAT
GTAGCACGTTGGTGTTTACGATGAATGCCTACTGCCTCGGA,
hMIFshRNA2-
SEQ ID NO: 15
TGCTGTTGACAGTGAGCGACGCGCAGAACCGCTCCTACAGTAGTGAAGCCACAGAT
GTACTGTAGGAGCGGTTCTGCGCGCTGCCTACTGCCTCGGA,
hMIFshRNA3-
SEQ ID NO: 16
TGCTGTTGACAGTGAGCGAAGGGTCTACATCAACTATTACTAGTGAAGCCACAGATG
TAGTAATAGTTGATGTAGACCCTGTGCCTACTGCCTCGGA,
mMIFshRNA1-
SEQ ID NO: 17
TGCTGTTGACAGTGAGCGCTCATCGTGAACACCAATGTTCTAGTGAAGCCACAGATG
TAGAACATTGGTGTTCACGATGAATGCCTACTGCCTCGGA,
mMIF shRNA2-
SEQ ID NO: 18
TGCTGTTGACAGTGAGCGAGCAGTGCACGTGGTCCCGGACTAGTGAAGCCACAGAT
GTAGTCCGGGACCACGTGCACTGCGTGCCTACTGCCTCGGA,
mMIF shRNA3-
SEQ ID NO: 19
TGCTGTTGACAGTGAGCGACGGGTCTACATCAACTATTACTAGTGAAGCCACAGATG
TAGTAATAGTTGATGTAGACCCGGTGCCTACTGCCTCGGA,
AIFshRNA1-
SEQ ID NO: 20
TGCTGTTGACAGTGAGCGCGGAACCGGCTTCCAGCTACAGTAGTGAAGCCACAGAT
GTACTGTAGCTGGAAGCCGGTTCCTTGCCTACTGCCTCGGA,
WT-mMIF-fw2-
SEQ ID NO: 21
CGGGATCCGCCACCATGCCTATGTTCATCGTGAAC,
WT-mMIF-re-
SEQ ID NO: 22
CGGAATTCTCAAGCGAAGGTGGAACCGT,
Rsh1-mMIF-fw-
SEQ ID NO: 23
CACCATGCCTATGTTTATTGTCAATACGAACGTACCCCGCGCCTCCGTG,
Rsh1-mMIF-re-
SEQ ID NO: 24
CACGGAGGCGCGGGGTACGTTCGTATTGACAATAAACATAGGCATGGTG,
Rsh3-mMIF-fw-
SEQ ID NO: 25
GCACATCAGCCCGGACCGCGTGTATATTAATTACTATGACATGAACGCTGCC,
Rsh3-mMIF-re-
SEQ ID NO: 26
GGCAGCGTTCATGTCATAGTAATTAATATACACGCGGTCCGGGCTGATGTGC,
hMIF P2G Fw-
SEQ ID NO: 27
GGGATCCCCGGAATTCggGATGTTCATCGTAAACACC,
hMIF P2G Re-
SEQ ID NO: 28
GGTGTTTACGATGAACATCCCGAATTCCGGGGATCCC,
P16A-hMIF-fw-
SEQ ID NO: 29
CCTCCGTGGCGGACGGGTTC,
P16A-hMIF-re-
SEQ ID NO: 30
GAACCCGTCCGCCACGGAGG,
D17A-hMIF-fw-
SEQ ID NO: 31
CGCGCCTCCGTGCCGGCCGGGTTCCTCTCC,
D17A-hMIF-re-
SEQ ID NO: 32
GGAGAGGAACCCGGCCGGCACGGAGGCGCG,
D17Q-hMIF-fw-
SEQ ID NO: 33
CCGTGCCGCAAGGGTTCCTC,
D17Q-hMIF-re-
SEQ ID NO: 34
GAGGAACCCTTGCGGCACGG,
E22A-hMIF-fw-
SEQ ID NO: 35
GGGTTCCTCTCCGCGCTCACCCAGCAGCTG,
E22A-hMIF-re-
SEQ ID NO: 36
CAGCTGCTGGGTGAGCGCGGAGAGGAACCC,
E22Q-hMIF-fw-
SEQ ID NO: 37
GGGTTCCTCTCCCAGCTCACCCAGCAGCTG,
E22Q-hMIF-re-
SEQ ID NO: 38
CAGCTGCTGGGTGAGCTGGGAGAGGAACCC,
E22D-hMIF-fw-
SEQ ID NO: 39
GGGTTCCTCTCCGACCTCACCCAGCAGCTG,
E22D-hMIF-re-
SEQ ID NO: 40
CAGCTGCTGGGTGAGGTCGGAGAGGAACCC,
P44A-hMIF-fw-
SEQ ID NO: 41
GTGCACGTGGTCGCGGACCA,
P44A-hMIF-re-
SEQ ID NO: 42
CATGAGCTGGTCCGCGACCA,
D45A-hMIF-fw-
SEQ ID NO: 43
GCACGTGGTCCCGGCCCAGCTCATGGCCTTC,
D45A-hMIF-re-
SEQ ID NO: 44
GAAGGCCATGAGCTGGGCCGGGACCACGTGC,
D45Q-hMIF-fw-
SEQ ID NO: 45
GTGGTCCCGCAACAGCTCAT,
D45Q-hMIF-re-
SEQ ID NO: 46
CCATGAGCTGTTGCGGGACC,
E55A-hMIF-fw2-
SEQ ID NO: 47
CTTCGGCGGCTCCAGCGCGCCGTGCGCGCTCTG,
E55A-hMIF-re2-
SEQ ID NO: 48
CAGAGCGCGCACGGCGCGCTGGAGCCGCCGAAG,
E55D-hMIF-fw-
SEQ ID NO: 49
CTCCAGCCAGCCGTGCGCGC,
E55D-hMIF-re-
SEQ ID NO: 50
GCGCGCACGGCTGGCTGGAG,
E86A-hMIF-fw-
SEQ ID NO: 51
GGCCTGCTGGCCGCGCGCCTGCGCATCAGC,
E86A-hMIF-re-
SEQ ID NO: 52
GCTGATGCGCAGGCGCGCGGCCAGCAGGCC,
R87Q-hMIF-fw-
SEQ ID NO: 53
GCTGGCCGAGCAACTGCGCATCAG,
R87Q-hMIF-re-
SEQ ID NO: 54
CTGATGCGCAGTTGCTCGGCCAGC,
R89Q-hMIF-fw-
SEQ ID NO: 55
CCGAGCGCCTGCAAATCAGC,
R89Q-hMIF-re-
SEQ ID NO: 56
GCTGATGCGCAGTTGCTCGG,
P92A-hMIF-fw-
SEQ ID NO: 57
GCGCATCAGCGCGGACAGGG,
P92A-hMIF-re-
SEQ ID NO: 58
CCCTGTCCGCGCTGATGCGC,
D93A-hMIF-fw2-
SEQ ID NO: 59
CTGCGCATCAGCCCGGCCAGGGTCTACATCAAC,
D93A-hMIF-re2-
SEQ ID NO: 60
GTTGATGTAGACCCTGGCCGGGCTGATGCGCAG,
D93Q-hMIF-fw-
SEQ ID NO: 61
CAGCCCGCAAAGGGTCTACA,
D93Q-hMIF-re-
SEQ ID NO: 62
TGTAGACCCTTTGCGGGCTG,
D101A-hMIF-fw-
SEQ ID NO: 63
CATCAACTATTACGCCATGAACGCGGCC,
D101A-hMIF-re-
SEQ ID NO: 64
GGCCGCGTTCATGGCGTAATAGTTGATG,
C57A,C60AhMIFfw-
SEQ ID NO: 65
CGGCGGCTCCAGCGAGCCGGCCGCGCTCGCCAGCCTGCACAGCATCGGC,
C57A,C60AhMIFre-
SEQ ID NO: 66
GCCGATGCTGTGCAGGCTGGCGAGCGCGGCCGGCTCGCTGGAGCCGCCG,
E22D-mMIF-fw-
SEQ ID NO: 67
GAGGGGTTTCTGTCGGACCTCACCCAGCAGCTG;
E22D-mMIF-re-
SEQ ID NO: 68
CAGCTGCTGGGTGAGGTCCGACAGAAACCCCTC;
E22Q-mMIF-fw-
SEQ ID NO: 69
GAGGGGTTTCTGTCGCAGCTCACCCAGCAGCTG;
E22Q-mMIF-re-
SEQ ID NO: 70
CAGCTGCTGGGTGAGCTGCGACAGAAACCCCTC;
AAV2-mMIFfw-
SEQ ID NO: 71
CGGATCCGCCACCATGCCTATGTTCATCGTG;
AAV2-mMIFre-
SEQ ID NO: 72
CGGAATTCTCACTTGTCGTCGTCGTCCTTGTAGTCAGCGAAGGTGGAACCGT;
hEndoG-fw-
SEQ ID NO: 73
CGGAATTCATGCGGGCGCTGCGGGCCGGCCT;
hEndoG-re-
SEQ ID NO: 74
CCGCTCGAGTCACTTACTGCCCGCCGTGATG;
hCypA-fw-
SEQ ID NO: 75
CGGAATTCATGGTCAACCCCACCGTGTTC;
hCypA-re-
SEQ ID NO: 76
CCGCTCGAGTTATTCGAGTTGTCCACAGTCAG;
EndoG gRNA1:
SEQ ID NO: 77
CCGCCGCCGCCAACCACCGC(TGG);
EndoG gRNA2:
SEQ ID NO: 78
GGGCTGGGTGCGGTCGTCGA(GGG)

The three letters in parentheses indicate the PAM sequence and the other sequences (20 nt) are target sites.

Cell Culture, Transfection, Lentiviral Transduction, and Cytotoxicity

HeLa cells were cultured in Dulbecco's modified Eagle's medium (Invitrogen) supplemented with 10% fetal bovine serum (HyClone). V5-tagged MIF was transfected with Lipofectamine Plus (Invitrogen). Primary neuronal cultures from cortex were prepared as previously described (9). Briefly, the cortex was dissected and the cells were dissociated by trituration in modified Eagle's medium (MEM), 20% horse serum, 30 mM glucose, and 2 mM L-glutamine after a 10-min digestion in 0.027% trypsin/saline solution (Gibco-BRL). The neurons were plated on 15-mm multiwell plates coated with polyornithine or on coverslips coated with polyornithine. Neurons were maintained in MEM, 10% horse serum, 30 mM glucose, and 2 mM L-glutamine in a 7% CO₂ humidified 37° C. incubator. The growth medium was replaced twice per week. In mature cultures, neurons represent 70 to 90% of the total number of cells. Days in vitro (DIV) 7 to 9, neurons were infected by lentivirus carrying MIF-WT-Flag, MIF-E22Q-Flag, or MIF-E22A-Flag [1×109 units (TU)/ml] for 72 hours. Parthanatos was induced by either MNNG (Sigma) in HeLa cells or NMDA (Sigma) in neurons. HeLa cells were exposed to MNNG (50 μM) for 15 min, and neurons (DIV 10 to 14) were washed with control salt solution [CSS, containing 120 mM NaCl, 5.4 mM KCl, 1.8 mM CaCl₂, 25 mM tris-Cl, and 20 mM glucose (pH 7.4)], exposed to 500 μM NMDA plus 10 μM glycine in CSS for 5 min, and then exposed to MEM containing 10% horse serum, 30 mM glucose, and 2 mM L-glutamine for various times before fixation, immunocytochemical staining, and confocal laser scanning microscopy. Cell viability was determined the following day by unbiased objective computer-assisted cell counting after staining of all nuclei with 7 μM Hoechst 33342 (Invitrogen) and dead cell nuclei with 2 μM propidium iodide (Invitrogen). The numbers of total and dead cells were counted with the Axiovision 4.6 software (Carl Zeiss). At least three separate experiments using at least six separate wells were performed with a minimum of 15,000 to 20,000 neurons or cells counted per data point. For neuronal toxicity assessments, glial nuclei fluoresced at a different intensity than neuronal nuclei and were gated out. The percentage of cell death was determined as the ratio of live to dead cells compared with the percentage of cell death in control wells to account for cell death attributed to mechanical stimulation of the cultures.

Pull-Down, Coimmunoprecipitation, and Immunoblotting

For the pull-down assay, GST-tagged MIF or AIF proteins immobilized glutathione Sepharose beads were incubated with 500 μg of HeLa cell lysates, washed in the lysis buffer, and eluted in the protein loading buffer. For coimmunoprecipitation, 1 mg whole-cell lysates were incubated overnight with AIF antibody (1 μg/ml) in the presence of protein A/G Sepharose (Santa Cruz Biotechnology), followed by immunoblot analysis with mouse anti-Flag antibody (Clone M1, Sigma), mouse anti-V5 (V8012, Sigma) or Goat anti-MIF (ab36146, Abcam). The proteins were separated on denaturing SDS-PAGE and transferred to a nitrocellulose membrane. The membrane was blocked and incubated overnight with primary antibody (50 ng/ml; mouse anti-Flag; rabbit anti-AIF; or goat anti-MIF) at 4° C., followed by horseradish peroxidase (HRP)-conjugated donkey anti-mouse, anti-rabbit or anti-goat for 1 hour at RT. After washing, the immune complexes were detected by the SuperSignalWest Pico Chemiluminescent Substrate (Pierce).

Subcellular Fraction

The nuclear extracts (N) and postnuclear cell extracts (PN), which is the fraction prepared from whole-cell lysates after removing nuclear proteins, were isolated in hypotonic buffer (9, 11). The integrity of the nuclear and postnuclear subcellular fractions was determined by monitoring histone H3 and MnSOD or Tom20 immunoreactivity (9, 11).

Immunocytochemistry, Immunohistochemistry, and Confocal Microscopy

For immunocytochemistry, cells were fixed 4 hours after MNNG or NMDA treatment with 4% paraformaldehyde, permeabilized with 0.05% Triton X-100, and blocked with 3% BSA in PBS. AIF was visualized by Donkey anti-Rabbit Cy3 or donkey anti-rabbit 647. MIF was visualized by donkey anti-mouse cy2 (2 μg/ml), donkey anti-goat Cy2 or donkey anti-goat 647. Immunohistochemistry was performed with an antibody against Flag. Immunofluorescence analysis was carried out with an LSM710 confocal laser scanning microscope (Carl Zeiss) as described (9).

FPLC

The native state and purity of the purified recombinant MIF were determined using standard calibration curve between elution volume and molecular mass (kDa) of known molecular weight native marker proteins in Akta Basic FPLC (Amersham-Pharmacia Limited) using Superdex 200 10/300GL column (GE Healthcare, Life Sciences). The gel filtration column was run in standard PBS buffer at a flow rate of 0.5 ml/min. The following molecular weight standards were used: Ferritin (440 kDa), aldolase (158 kDa), conalbumin (75 kDa), ovalbumin (43 kDa), carbonic anhydrase (29 kDa), and ribonuclease (13.7 kDa) respectively (GE Healthcare, Life Sciences). Eluted fractions containing MIF was resolved on 12% SDS-PAGE and stained with commassie blue to check the purity of the protein.

Mass Spectrometry Analysis for MIF Protein Purity

MIF proteins used for nuclease assays were also examined by mass spectrometry in order to exclude any possible contamination from other known nucleases. Analyses using different criteria at a 95% and lower confidence levels were performed in order to capture any remote possibility of a nuclease. Analysis and search of the NCBI database using all species reveal that no known nuclease that can digest single or double-stranded DNA was detected in the MIF protein that was used in the nuclease assays.

Circular Dichroism (CD) Spectroscopy

CD spectroscopy was performed on a AVIV 420 CD spectrometer (Biomedical Inc., Lakewood, N.J., USA). Near-UV CD spectra were recorded between 240-320 nm using a quartz cuvette of 0.5 cm path length with protein samples at a concentration of 2 mg/ml at room temperature. Far UV CD spectra were also recorded at room temperature between 190-260 nm using quartz sandwich cuvettes of 0.1 cm path length with protein samples at a concentration of 0.2 mg/ml (41). The proteins were suspended in PBS buffer with or without magnesium chloride (5.0 mM) and/or zinc chloride (0.2 mM). The CD spectra were obtained from 0.5 nm data pitch, 1 nm/3 sec scan speed and 0.5 s response time were selected for the recordings.

Oxido-Reductase Activity Assay

The thiol-protein oxidoreductase activity of MIF was measured using insulin as the substrate as described previously (30). Briefly, the insulin assay is based on the reduction of insulin and subsequent insolubilization of the insulin β-chain. The time-dependent increase in turbidity is then measured spectrophotometrically at 650 nm. The reaction was started by adding 5 μM MIF WT, E22A, E22Q, C57A; C60A or and P2G mutants dissolved in 20 mM sodium phosphate buffer (pH 7.2), and 200 mM reduced glutathione (GSH) to ice-cold reaction mixture containing 1 mg/ml insulin, 100 mM sodium phosphate buffer (pH 7.2) and 2 mM EDTA. MIF insulin reduction was measured against the control solution (containing GSH) in the same experiment.

Tautomerase Activity Assay

Tautomerase activity was measured using the D-dopachrome tautomerase as the substrate as described previously (42). Briefly, a fresh solution of D-dopachrome methyl ester was prepared by mixing 2 mM L-3,4 dihydroxyphenylalanine methyl ester with 4 mM sodium peroxidate for 5 min at room temperature and then placed directly on ice before use. The enzymatic reaction was initiated at 25° C. by adding 20 μl of the dopachrome methyl ester substrate to 200 μl of MIF WT, E22A, E22Q, C57A; C60A (final concentration 5 μM) or and P2G mutants prepared in tautomerase assay buffer (50 mM potassium phos¬phate, 1 mM EDTA, pH 6.0). The activity was determined by the semi-continuous reduction of OD 475 nm using a spectrophotometer.

Intracerebroventricular (ICV) Injection

3 μl AAV2-MIF WT, E22Q and E22A (1×1013 GC/ml, Vector BioLabs) were injected into both sides of intracerebroventricular of the newborn MIF KO mice (34). The expression of MIF and its variants were checked by immunohistochemistry after MCAO surgery during the age: 8-16 week.

Neurobehavioral Activity

Spontaneous motor activity was evaluated 1 day, 3 days and 7 days after MCAO by placing the animals in a mouse cage for 5 minutes. A video camera was fitted on top of the cage to record the activity of a mouse in the cage. Neurological deficits were evaluated by an observer blinded to the treatment and genotype of the animals with a scale of 0-5 (0, no neurological deficit; 5, severe neurological deficit). The following criteria were used to score deficits: 0=mice appeared normal, explored the cage environment and moved around in the cage freely; 1=mice hesitantly moved in cage but could occasionally touch the walls of the cage, 2=mice showed postural and movement abnormalities, and didn't approach all sides of the cage, 3=mice showed postural and movement abnormalities and made medium size circles in the center of cage, 4=mice with postural abnormalities and made very small circles in place, 5=mice were unable to move in the cage and stayed at the center. Recordings were evaluated by observers blinded to the treatment and genotype of the animals.

The corner test was performed 1 day, 3 days and 7 days after MCAO to assess sensory and motor deficits following both cortical and striatal injury. A video camera was fitted on top of the cage to record the activity of a mouse in the cage for 5 min. The mice were placed between two cardboards each with a dimension of 30 cm×20 cm×0.5 mm attached to each other from the edges with an angle of 30°. Once in the corner, the mice usually rear and then turn either left or right. Before stroke mice do not show a side preference. Mice with sensory and motor deficits following stroke will turn toward the non-impaired side (right). % of right turn=right turns/total turns×100 was calculated and compared. Recordings were evaluated by observers blinded to the treatment and genotype of the animals.

Animals

The Johns Hopkins Medical Institutions are fully accredited by the American Association for the Accreditation of Laboratory Animal Care (AAALAC). All research procedures performed in this study were approved the Johns Hopkins Medical Institutions Institutional Animal Care and Use Committee (IACUC) in compliance with the Animal Welfare Act regulations and Public Health Service (PHS) Policy. All animal studies were performed in a blinded fashion. Mouse genotype was determined by K.N. Stroke surgery was performed by R.A. Mouse genotypes were decoded after the stroke surgery, mouse behavior tests and data analysis. Based on their genotype, mice were grouped as WT, KO, KO-WT, KO-E22Q and KO-E22A. Within each group, mice were randomly assigned to subgroups including sham, 1 day-post stroke, 3 days- or 7 days-post stroke.

Statistical Analysis

Unless otherwise indicated, statistical evaluation was carried out by Student's t test between two groups and by one-way analysis of variance (ANOVA) followed by post hoc comparisons with the Bonferroni test using GraphPad Prism software within multiple groups. Data are shown as means±SEM. P<0.05 is considered significant. Experiments for quantification were performed in a blinded fashion. In order to ensure adequate power to detect the effect, at least 3 independent tests were performed for all molecular biochemistry studies and at least five mice from three different litters were used for animal studies.

Additional analysis of EndoG, MIF protein structure, MIF mutations, MIF protein purity, ChIP sequencing data and MIF-AIF interaction.

EndoG is Dispensable for PARP-1 Dependent Cell Death

To confirm that the EndoG is dispensable for parthanatos as previously described (13, 14), the CRISPR/Cas9 system was used to knockout endoG from human SH-SY5Y cells (FIG. 12A). It was found that knockout of endoG failed to block MNNG induced parthanatos (FIG. 12B) and large DNA fragmentation (FIG. 12C), confirming that EndoG is not required for parthanatos (13, 14).

Analysis of MIF Protein Structure

The core PD-D/E(X)K topology structure of nucleases consists of 40-strands next to two-helices (18). Two of the β-strands are parallel to each other whereas the other two are antiparallel (FIG. 14C, modified from (18)). Previous 3-D crystal structures of MIF indicate that it exists as a trimer (21-23). The trimeric structure of MIF enables the interaction of the β-strands of each monomer with the other monomers resulting in a PD-D/E(X)K structure that consists of 4 β-strands next to 2 α-strands (FIG. 1E and FIG. 14, D to G). Two of the β-strands (β-4 and β-5) are parallel whereas the other two strands (β-6 and β-7) (from the adjacent monomer) are anti-parallel (FIG. 14, D to G). The topology structure of PD-D/E(X)K motifs with orientations of the beta-strands relative to the alpha helices in the MIF trimer are very similar to EcoRV, a well characterized endonuclease (FIG. 14, H to K). Importantly, the PD-D/E(X)K motif based on the trimer structure of MIF is structurally similar to type II ATP independent restriction endonucleases, such as EcoRI and EcoRV, as well as, ExoIII family purinic/apyrimidinic (AP) endonucleases, such as ExoIII (FIG. 1E and FIG. 14, L to N). Moreover, MIF also has a similar topology to the PvuII endonuclease and its β-7 strand is of similar size to PvuII endonuclease 0-strand at the same position in its PD-D/E(x)K motif (FIG. 14O). These 3-D modeling results taken together indicate that MIF belongs to the PD-D/E(X)K nuclease-like superfamily (24, 25).

Identification of Key Residues Critical for MIF's Nuclease Activity

To identify amino acid residues critical for MIF's nuclease activity, key aspartates and glutamates residues within the PD-D/E(X)K domains of MIF were mutated to alanine. Substitution of glutamate 22 by alanine (E22A) clearly but not completely reduces MIF's nuclease activity, whereas alanine substitutions at the other aspartates and glutamates including D17A, D45A, E55A, E86A, D93A and D101A have no substantial effect (FIG. 15E). Mutation of the CxxCxxHx(n)C zinc finger domain of MIF to C57A; C60A has no appreciable effect (FIG. 15E). Since MIF E22A has reduced nuclease activity, additional conserved mutations around E22 were made (FIGS. 15, F and G). It was found that MIF E22Q has no nuclease activity (FIG. 2D and FIGS. 15,D and H), whereas E22D has equivalent nuclease activity to wild type (FIG. 2D). These data suggest that this glutamic acid residue (E22) in the first α-helix of MIF is critical for its nuclease activity, which is consistent with prior reports that this glutamic acid in the first α-helix of many Exonuclease-Endonuclease-Phosphatase (EEP) domain superfamily nucleases is highly conserved and it is the active site for nuclease activity (24, 25). Based on 3-dimensional structural modeling (FIG. 1E), possible MIF DNA binding sites were mutated including: P16A, P44A, R87Q, R89Q, P92A, D45Q, D17Q, E55Q, and D93Q (FIG. 15H). It was found that P16A or D17Q prevents MIF nuclease activity (FIG. 15H). Based on both the sequence alignment and 3-dimensional structural modeling of MIF, the data reveal that P16, D17 and E22 are within the same PD-D/E(X)K motif and mutation of each single residue is sufficient to block MIF nuclease activity. Considering the fact that E22 in the first α-helix is highly conserved across species and previously it has been reported as an active site for PD-D/E(X)K nuclease-like superfamily nuclease activity (24, 25), the E22 mutant was focused on in subsequent studies.

EndoG and Cyclophilin A are not Directly Involved in PARP-1 Dependent Large DNA Fragmentation

EndoG and cyclophilin A have been previously suggested to be AIF associated nucleases (43-45). Pulsed-field gel electrophoresis indicates that EndoG cleaves DNA into small fragments that are not consistent with the larger DNA fragmentation pattern observed in parthanatos (FIG. 15D). In contrast, MIF cleaves DNA into large fragments with a pattern similar to MNNG induced DNA fragments (FIG. 2B and FIG. 15D) (13, 14). Cyclophilin A and AIF have no obvious nuclease activity with glutathione S-transferase (GST) serving as a negative control (FIG. 15D).

MIF Nuclease Activity is Independent of its Oxidoreductase and Tautomerase Activities

Previous studies indicate that MIF has both oxidoreductase and tautomerase activities (27, 29, 30). The oxidoreductase activity of wild type and MIF mutants were measured using insulin as a substrate in which reduced insulin exhibits an optical density value of 650 nm in the presence of wild type MIF (FIG. 16A). E22Q, E22A, C57A; C60A MIF mutants and the tautomerase P2G MIF mutant have no appreciable effects on MIF's oxidoreductase activity (FIG. 16A). MIF's tautomerase activity was also measured. E22Q, E22A, C57A; C60A MIF mutations have no appreciable effect on MIF's tautomerase activity whereas the P2G MIF mutant significantly reduces MIF's tautomerase activity (FIG. 16B). These results taken together indicate that MIF active site mutants E22Q and E22A have no appreciable effect on MIF's oxidoreductase or tautomerase activities.

Purified MIF Proteins have No Adventitious Nuclease Contamination

To confirm that the recombinant MIF preparations did not contain an adventitious nuclease, FPLC was performed. FPLC reveals only one peak at a molecular weight of approximately 37 kD consistent with MIF existing as a trimer. MIF E22Q and E22A also elute at 37 kD consistent with a trimer structure suggesting that these mutations do not appreciably affect the confirmation of MIF (FIG. 16C). Coomassie blue staining reveals only a single band in the proteins following FPLC purification (FIG. 16D) as well as proteins without FPLC purification (FIG. 15G). Both types of proteins with and without FPLC purification were used in the nuclease assays and no obvious difference was observed. The purity of all these recombinant MIF proteins used in the nuclease assays was also confirmed by two-independent mass spectrometry (MS) assays. The majority of peptides identified by MS are MIF. No known nuclease from all species that can cleave single or double stranded DNA was identified. Thus, MIF preparations are highly pure and no adventitious nuclease is present.

MIF Protein Confirmation is Unaffected by E22Q, E22A and C57A; C60A Mutations

Far-ultraviolet (UV) circular dichroism (CD) spectroscopy, a common method to study protein secondary structure shows that wild type MIF is composed of a mixture of α-helices and Q-sheets in agreement with the previously reported crystal structure of MIF (22). MIF mutants, E22Q, E22A and C57A; C60A, show similar CD spectra as wild type MIF suggesting that these mutations do not significantly affect the conformation of MIF (FIG. 16E). No significant change is observed on the addition of Mg²⁺ to wild type MIF or MIF mutants (FIG. 16F-I). However, addition of Zn²⁺ promotes large changes in the spectra indicating significant changes in the structure of the wild type MIF protein on Zn²⁺ binding (FIG. 16F). MIF E22Q and E22A show a similar CD spectra as wild type MIF in the presence of Zn²⁺ (FIGS. 16, G and H), however the addition of Zn²⁺ to the C57A; C60A mutant did not cause a change in the CD spectra indicating that MIF binds Zn²⁺ at the CxxCxxHx(n)C zinc finger domain of MIF (FIG. 16I).

Near UV CD spectroscopy was used to further analyze the tertiary structure of MIF and MIF mutants. The purified proteins have a properly folded tertiary structure since there are distinct peaks of phenylalanine and tyrosine in the near UV CD spectra (FIG. 16J). MIF mutants, E22Q, E22A and C57A; C60A, show similar near UV CD spectra as wild type suggesting that these mutations do not significantly affect the tertiary structure of MIF (FIG. 16, J to M). The addition of Mg²⁺ to wild type MIF or the MIF mutant C57A; C60A causes a significant change in tertiary structure indicative of Mg²⁺ binding (FIGS. 16, J and M). The E22A shows minor changes in the presence of Mg²⁺ and the E22Q shows no significant changes in the near UV CD spectra suggesting that Mg²⁺ binds at or near E22 (FIGS. 16, K and L), which is consistent with our finding that Mg²⁺ is required for MIF's nuclease activity and E22Q and E22A mutants can block its nuclease activity completely or partially. The addition of Zn²⁺ to wild type MIF or MIF mutants E22A and E22Q causes a significant change in the tertiary structure indicative of Zn²⁺ binding whereas the MIF C57A; C60A mutant exhibits no significant change consistent with the Zn²⁺ binding to the CxxCxxHx(n)C zinc finger domain (FIG. 16, J to M).

ChIP Sequencing Analysis of MIF-DNA Binding Properties

Because the data shows that MIF has nuclease activity, next whether MIF binds to DNA in HeLa cells treated with DMSO or MNNG (50 μM, 15 min) was studied. Five hours after the treatment, cells were cross-linked and prepared for ChTP assays followed by deep sequencing. The quality of sheared genomic DNA and the specificity of ChTP using the MIF antibody was tested and confirmed (FIGS. 17, A and B). After excluding overlapped peaks in the DMSO-treated samples, 0.1% of total mapped reads exhibit MIF peaks after MNNG treatment (FIG. 17C). MIF preferentially binds to the promoter and 5′ UTR regions after MNNG treatment (FIG. 17D). The representative IGV visualization of MIF enrichment on the genome is shown in two different window sizes (250 kb (FIG. 17E) and 50 kb (FIG. 17F). The average distance intervals between MIF peaks are about 15 to 60 kb, which is consistent with size of DNA fragments observed via pulse-gel electrophoresis during parthanatos. ChIP-qPCR further confirms that MIF binds to the peak regions at 55101, 66005, 65892, 36229, 46426 and 62750 but it does not bind to the non-peak regions after MNNG treatment (FIG. 17G).

Mapping AIF-MIF Interactions

To confirm that MIF is an AIF interacting protein, GST pull down experiments were performed. Wild type GST-AIF pulls down endogenous MIF and wild type GST-MIF pulls down endogenous AIF (FIG. 4A and FIG. 21, A to D). The domain that binds MIF was further defined by GST pull downs with various GST-tagged AIF domains (FIG. 21A). MIF binds to GST-C2b AIF (aa 551-590) and GST C2e AIF (aa 571-612) (FIGS. 21, A and B). MIF does not bind to GST-C2aAIF, GST-C2cAIF, GST-C2dAIF or GST indicating that it does not nonspecifically bind to GST at the experimental conditions used (FIGS. 21, A and B). Mutating aa567-592 into polyalanines (AIFm567-592) or deleting aa567-592 (AIFA567-592) from full length completely abolished MIF and AIF binding (FIG. 21C), suggesting that MIF binds to AIF at aa 567-592.

Prior crystallization studies of MIF demonstrated via 3-D modeling that MIF is structurally similar to PD-D/E(x)K nucleases. Proteins containing PD-D/E(X)K domains belong to the nuclease-like superfamily (for review see (24, 25)), providing further evidence that MIF is a nuclease. This nuclease superfamily contains nucleases from all kingdoms of life. The majority of these proteins belong to prokaryotic organisms, but this domain is contained with a variety of vertebrate nucleases (24, 25). The PD-D/E(X)K domains in MIF are highly conserved in vertebrates. The glutamic acid residue (E22) in the first α-helix of MIF is critical for its nuclease activity, which is consistent with prior reports that this glutamic acid in the first α-helix of many Exonuclease-Endonuclease-Phosphatase (EEP) domain superfamily nucleases is highly conserved and it is the active site for nuclease activity (24, 25).

The core PD-D/E(x)K structure consists of 4 β-strands next to two α-helices. Two of the β-strands are parallel to each other whereas the other two are antiparallel (18, 24). Interestingly, the MIF monomer, which has pseudo 2-fold symmetry does not contain the core PD-D/E(x)K structure since the MIF monomer has 4 β-strands next to the 2 α-helices, and the orientations of the β-strands within an isolated monomer do not fit the requirement of the PD-D/E(x)K topology (22). However, the structure-activity analyses based on the MIF trimer, which has 3-fold symmetry indicate that the interactions of the β-strands of each monomer with the other monomers results in a MIF PD-D/E(x)K structure that consists of 4 β-strands next to 2 α-strands (22). Two of the β-strands are parallel (β-4 and β-5) whereas the other two strands (β-6 and β-7) (from the adjacent monomer) are anti-parallel. This topology exquisitely supports the idea that MIF's nuclease activity requires the trimer as the monomers do not support the required topology and is consistent with MIF existing as a trimer. This topology of the MIF trimer places the α-1 helix, which contains the active residue, glutamate 22, next to the β-strands, but this is not unprecedented (18, 24). For example, EcoRV, a well characterized endonuclease has PD-D/E(x)K motifs with orientations of the beta-strands relative to the alpha helices different from the classical PD-D/E(x)K motif and similar to that of MIF. The similarity in the topology of MIF versus EcoRV suggests that MIF is highly similar to the well characterized restriction endonucleases. Indeed conserved acidic residues from the core α-helices (usually) glutamic acid from the first α-helix often contributes to active site formation at least in a subset of PD-D/E(x)K families similar to what was reported for MIF (24). The PD-D/E(x)K motif based on MIF's trimer structure also has a very similar structure to the nucleases ExoIII, EcoRI and EcoRV. Moreover, MIF has a similar topology to the PvuII endonuclease and MIF's β-7 strand is of similar size to PvuII endonuclease Q-strand at the same position in its PD-D/E(x)K motif (46). Based on the structural analysis, MIF should be classified as nuclease.

MIF has a variety of pleiotropic actions. It functions as a non-classically secreted cytokine where it may play important roles in cancer biology, immune responses and inflammation (16, 17). MIF also has important roles in cellular stress and apoptosis (47, 48). Knockout of MIF has also been shown to be neuroprotective in focal ischemia (49). The results confirm that knockout of MIF protects against focal ischemia and shows that MIF contributes to the neuronal damage in focal ischemia via its binding to AIF and its nuclease activity consistent with its function as a PAAN. MIF also has thiol-protein oxidoreductase activity and tautomerase activity. Both EMSA and ChTP indicate that MIF binds DNA. Although MIF binds a highly related family of overlapping sequences, the structure-activity experiment indicates that MIF preferentially binds to ssDNA based on its structure and that it relies less on sequence specificity. MIF binds at 5′ unpaired bases of ssDNA with stem loop structure and it has both 3′ exonuclease and endonuclease activities and cleaves unpaired bases at the 3′ end of stem loop ssDNA. The 3-dimensional computational modeling shows that the catalytic E22 is close to the modeled binding domain of ssDNA. As shown here, MIF's nuclease activity is clearly separable from it oxidoreductase and tautomerase activities.

Previous attempts to identify the AIF associated nuclease initially focused on EndoG, a mitochondrial matrix protein (43). In C. elegans, CPS-6 the homolog of mammalian EndoG is required for WAH-1's (AIF homolog) cell death inducing properties. However, in mammals, EndoG is dispensable in many models of cell death including PARP-1 dependent ischemic cell death (13, 14, 50). Importantly there was an equivalent amount of DNA fragmentation in EndoG knockout mice compared to wild type controls following middle cerebral artery occlusion (13). Consistent with these observations, it was confirmed that knockout of endoG failed to block MNNG induced parthanatos and large DNA fragmentation confirming that EndoG is not required for parthanatos (13, 14). In contrast, knockout of MIF, a MIF nuclease-deficient mutant and a MIF AIF binding deficient mutant prevent cell death and large DNA fragmentation both in vitro and in vivo following activation of PARP-1. Thus, EndoG is not the PAAN in mammals, whereas MIF fits all the criteria for this role. Recently, it was suggested that AIF generates an active DNA degrading complex with cyclophilin A (45), but the nuclease in this complex was not identified.

TABLE 1
Summary of MIF substrate used for the nuclease assays. (Y:yes; N:no)

| | | | Loop | Endo- | Exo- |
|---|---|---|---|---|---|
| | | | sequence | Nuclease | Nuclease |
| Name | Sequence | Loop | same | Activity | Activity |
| PS³⁰ | /5Biosg/CTCAGCCTCCCAAGTAGCTG | Y | Y | Y | Y |
| | GGATTACAGG SEQ ID NO: 5 | | | | |
| PS⁴⁰ | /5Biosg/CTCAGCCTCCCAAGTAGCTG | Y | N | Y | Y |
| | GGATTACAGGTAAACTTGGT SEQ | | | | |
| | ID NO: 11 | | | | |
| 3F1 | /5Biosg/aaaaaaaaaaCAAGTAGCTGGGA | Y | N | Y | Y |
| | TTACAGG SEQ ID NO: 12 | | | | |
| m2 | /5Biosg/CTCAGCCTCCaaaaaaaaaaGGA | Y | N | Y | Y |
| | TTACAGG SEQ ID NO: 79 | | | | |
| m3 | /5Biosg/CTCAGCCTCCCAAGTAGCTG | Y | N | Y | Y |
| | aaaaaaaaaa SEQ ID NO: 80 | | | | |
| m4 | /5Biosg/CTCAGCCaaaaAAaTAGCTGG | Y | N | Y | Y |
| | GATTACAGG SEQ ID NO: 81 | | | | |
| m5 | /5Biosg/CTCAGCCTCCCAAaaAaaaGG | Y | N | Y | Y |
| | GATTACAGG SEQ ID NO: 82 | | | | |
| m6 | /5Biosg/CTCAaaaaaaCAAGTAGCTGGG | Y | N | Y | Y |
| | ATTACAGG SEQ ID NO: 83 | | | | |
| m7 | /5Biosg/aaaaGCCTCCCAAGTAGCTGG | Y | Y | Y | Y |
| | GATTACAGG SEQ ID NO: 84 | | | | |
| m8 | /5Biosg/CTCAaaaTCCCAAaaAaaaGGG | Y | N | Y | Y |
| | ATTACAGG SEQ ID NO: 85 | | | | |
| m9 | /5Biosg/CAAGTAGCTGCTCAGCCTCC | Y | N | Y | Y |
| | GGATTACAGG SEQ ID NO: 86 | | | | |
| m10 | /5Biosg/CTCAGCCTCCGGATTACAGG | Y | N | Y | Y |
| | CAAGTAGCTG SEQ ID NO: 87 | | | | |
| m11 | /5Biosg/CTCAGCCTCCCAAGTAaaaGG | Y | N | Y | Y |
| | GATTACAGG SEQ ID NO: 88 | | | | |
| m12 | /5Biosg/CTCAGCCTCCCAAGTAaaTG | Y | N | Y | Y |
| | GGATTACAGG SEQ ID NO: 89 | | | | |
| m14 | /5Biosg/CTCAGCCTCCCAAGTAaacGG | Y | N | Y | Y |
| | GATTACAGG SEQ ID NO: 90 | | | | |
| m15 | /5Biosg/CTCAGCCTCCCAAGTAGCaG | Y | N | Y | Y |
| | GGATTACAGG SEQ ID NO: 91 | | | | |
| m16 | /5Biosg/CTCAGCCTCCCttGTAGCTGG | Y | N | Y | Y |
| | GATTACAGG SEQ ID NO: 92 | | | | |
| m17 | /5Biosg/CTCAaaaTCCCAAGTAGCTGG | Y | Y | Y | Y |
| | GATTACAGG SEQ ID NO: 93 | | | | |
| m18 | /5Biosg/CTCAattTCCCAAGTAGCTGG | Y | Y | Y | Y |
| | GATTACAGG SEQ ID NO: 94 | | | | |
| SL | /5Biosg/CctgtaaTCCCAAGTAGCTGGG | Y | Y | N | N |
| | ATTACAGG SEQ ID NO: 6 | | | | |
| LF | /5Biosg/aaaaaaaCTCAGCCTCCCAAGT | Y | Y | N | N |
| | AGCTGGGA SEQ ID NO: 7 | | | | |
| m20 | /5Biosg/aaaaatCTCAGCCTCCCAAGTA | Y | N | Y | Y |
| | GCTGGGAT SEQ ID NO: 95 | | | | |
| RF | /5Biosg/TCCCAAGTAGCTGGGATTAC | Y | N | Y | Y |
| | AGGaaaaaaa SEQ ID NO: 8 | | | | |
| BS2 | /5Biosg/TGGGATTACAGGCGTGAGC | Y | N | Y | Y |
| | CACCACGCCC SEQ ID NO: 96 | | | | |
| PA³⁰ | /5Biosg/AAAAAAAAAAAAAAAAAAA | N | — | N | Y |
| | AAAAAAAAAAA SEQ ID NO: 9 | | | | |
| 3E | 5′CTCAGCCTCCCAAGTAGCTGGGAT | N | — | N | Y |
| | TACAGG3′ SEQ ID NO: 5; | | | | |
| | 5′TCCCAGCTACTTGGGAGGCTGAG3′ | | | | |
| | SEQ ID NO: 10 | | | | |
| L3 | /5Biosg/ACCTAAATGCTAGAGCTCGC | Y | N | Y | Y |
| | TGATCAGCCT SEQ ID NO: 13 | | | | |

Although the invention has been described with reference to the above examples, it will be understood that modifications and variations are encompassed within the spirit and scope of the invention. Accordingly, the invention is limited only by the following claims.

### REFERENCES

The following references are each relied upon and incorporated herein in their entirety.
1. P. Bai, Biology of Poly(ADP-Ribose) Polymerases: The Factotums of Cell Maintenance. Mol Cell 58, 947 (Jun. 18, 2015).
2. A. A. Fatokun, V. L. Dawson, T. M. Dawson, Parthanatos: mitochondrial-linked mechanisms and therapeutic opportunities. Br J Pharmacol 171, 2000 (April, 2014).
3. Y. Wang, V. L. Dawson, T. M. Dawson, Poly(ADP-ribose) signals to mitochondrial AIF: a key event in parthanatos. Exp Neurol 218, 193 (August, 2009).
4. P. Pacher, C. Szabo, Role of the peroxynitrite-poly(ADP-ribose) polymerase pathway in human disease. Am J Pathol 173, 2 (July, 2008).
5. C. Szabo, V. L. Dawson, Role of poly(ADP-ribose) synthetase in inflammation and ischaemia-reperfusion. Trends Pharmacol Sci 19, 287 (July, 1998).
6. S. Martire, L. Mosca, M. d′Erme, PARP-1 involvement in neurodegeneration: A focus on Alzheimer's and Parkinson's diseases. Mech Ageing Dev 146-148C, 53 (March, 2015).
7. L. Virag, A. Robaszkiewicz, J. M. Rodriguez-Vargas, F. J. Oliver, Poly(ADP-ribose) signaling in cell death. Mol Aspects Med 34, 1153 (December, 2013).
8. H. Wang et al., Apoptosis-inducing factor substitutes for caspase executioners in NMDA-triggered excitotoxic neuronal death. J Neurosci 24, 10963 (Dec. 1, 2004).
9. Y. Wang et al., Poly(ADP-ribose) (PAR) binding to apoptosis-inducing factor is critical for PAR polymerase-1-dependent cell death (parthanatos). Sci Signal 4, ra20 (2011).
10. S. W. Yu et al., Apoptosis-inducing factor mediates poly(ADP-ribose) (PAR) polymer-induced cell death. Proc Natl Acad Sci USA 103, 18314 (Nov. 28, 2006).
11. S. W. Yu et al., Mediation of poly(ADP-ribose) polymerase-1-dependent cell death by apoptosis-inducing factor. Science 297, 259 (Jul. 12, 2002).
12. X. Wang, C. Yang, J. Chai, Y. Shi, D. Xue, Mechanisms of AIF-mediated apoptotic DNA degradation in Caenorhabditis elegans. Science 298, 1587 (Nov. 22, 2002).
13. Z. Xu et al., Endonuclease G does not play an obligatory role in poly(ADP-ribose) polymerase-dependent cell death after transient focal cerebral ischemia. Am J Physiol Regul Integr Comp Physiol 299, R215 (July, 2010).
14. K. K. David, M. Sasaki, S. W. Yu, T. M. Dawson, V. L. Dawson, EndoG is dispensable in embryogenesis and apoptosis. Cell Death Differ 13, 1147 (July, 2006).
15. S. Hu et al., Profiling the human protein-DNA interactome reveals ERK2 as a transcriptional repressor of interferon signaling. Cell 139, 610 (Oct. 30, 2009).
16. T. Calandra, T. Roger, Macrophage migration inhibitory factor: a regulator of innate immunity. Nat Rev Immunol 3, 791 (October, 2003).
17. M. Merk, R. A. Mitchell, S. Endres, R. Bucala, D-dopachrome tautomerase (D-DT or MIF-2): doubling the MIF cytokine family. Cytokine 59, 10 (July, 2012).
18. J. Kosinski, M. Feder, J. M. Bujnicki, The PD-(D/E)XK superfamily revisited: identification of new members among proteins involved in DNA metabolism and functional predictions for domains of (hitherto) unknown function. BMC Bioinformatics 6, 172 (2005).
19. C. MacKay et al., Identification of KIAA1018/FAN1, a DNA repair nuclease recruited to DNA damage by monoubiquitinated FANCD2. Cell 142, 65 (Jul. 9, 2010).
20. K. Kratz et al., Deficiency of FANCD2-associated nuclease KIAA1018/FAN1 sensitizes cells to interstrand crosslinking agents. Cell 142, 77 (Jul. 9, 2010).
21. H. Sugimoto, M. Suzuki, A. Nakagawa, I. Tanaka, J. Nishihira, Crystal structure of macrophage migration inhibitory factor from human lymphocyte at 2.1 A resolution. FEBS Lett 389, 145 (Jul. 1, 1996).
22. H. W. Sun, J. Bernhagen, R. Bucala, E. Lolis, Crystal structure at 2.6-A resolution of human macrophage migration inhibitory factor. Proc Nat Acad Sci USA 93, 5191 (May 28, 1996).
23. M. Suzuki et al., Crystal structure of the macrophage migration inhibitory factor from rat liver. Nat Struct Biol 3, 259 (March, 1996).
24. K. Steczkiewicz, A. Muszewska, L. Knizewski, L. Rychlewski, K. Ginalski, Sequence, structure and functional diversity of PD-(D/E)XK phosphodiesterase superfamily. Nucleic Acids Res 40, 7016 (August, 2012).
25. M. Laganeckas, M. Margelevicius, C. Venclovas, Identification of new homologs of PD-(D/E)XK nucleases by support vector machines trained on data derived from profile-profile alignments. Nucleic Acids Res 39, 1187 (March, 2011).
26. V. Pingoud et al., On the divalent metal ion dependence of DNA cleavage by restriction endonucleases of the EcoRI family. J Mol Biol 393, 140 (Oct. 16, 2009).
27. J. B. Lubetsky et al., The tautomerase active site of macrophage migration inhibitory factor is a potential target for discovery of novel anti-inflammatory agents. J Biol Chem 277, 24976 (Jul. 12, 2002).
28. G. Fingerle-Rowson et al., A tautomerase-null macrophage migration-inhibitory factor (MIF) gene knock-in mouse model reveals that protein interactions and not enzymatic activity mediate MIF-dependent growth regulation. Mol Cell Biol 29, 1922 (April, 2009).
29. E. Rosengren et al., The macrophage migration inhibitory factor MIF is a phenylpyruvate tautomerase. FEBS Lett 417, 85 (Nov. 3, 1997).
30. A. Kudrin et al., Human macrophage migration inhibitory factor: a proven immunomodulatory cytokine? J Biol Chem 281, 29641 (Oct. 6, 2006).
31. G. Zhao, B. Zhao, Z. Tong, R. Mu, Y. Guan, Effects of 2′-O-methyl nucleotide substitution on EcoRI endonuclease cleavage activities. PLoS One 8, e77111 (2013).
32. M. Bozza et al., Targeted disruption of migration inhibitory factor gene reveals its critical role in sepsis. J Exp Med 189, 341 (Jan. 18, 1999).
33. S. A. Andrabi et al., Iduna protects the brain from glutamate excitotoxicity and stroke by interfering with poly(ADP-ribose) polymer-induced cell death. Nat Med 17, 692 (June, 2011).
34. J. J. Glascock et al., Delivery of therapeutic agents through intracerebroventricular (ICV) and intravenous (IV) injection in mice. J Vis Exp, (2011).
35. Y. Chen et al., Systematic evaluation of factors influencing ChIP-seq fidelity. Nat Methods 9, 609 (June, 2012).
36. J. Feng, T. Liu, B. Qin, Y. Zhang, X. S. Liu, Identifying ChIP-seq enrichment using MACS. Nat Protoc 7, 1728 (September, 2012).
37. H. R. Drew et al., Structure of a B-DNA dodecamer: conformation and dynamics. Proc Nat Acad Sci USA 78, 2179 (April, 1981).
38. T. Masuda, Y. Ito, T. Terada, T. Shibata, T. Mikawa, A non-canonical DNA structure enables homologous recombination in various genetic systems. J Biol Chem 284, 30230 (Oct. 30, 2009).
39. A. W. Ghoorah, M. D. Devignes, M. Smail-Tabbone, D. W. Ritchie, Protein docking using case-based reasoning. Proteins, (Oct. 7, 2013).
40. G. Macindoe, L. Mavridis, V. Venkatraman, M. D. Devignes, D. W. Ritchie, HexServer: an FFT-based protein docking server powered by graphics processors. Nucleic Acids Res 38, W445 (July, 2010).
41. P. P. Reddy et al., Molecular dynamics of the neuronal EF-hand Ca2+-sensor Caldendrin. PLoS One 9, e103186 (2014).
42. K. Bendrat et al., Biochemical and mutational investigations of the enzymatic activity of macrophage migration inhibitory factor. Biochemistry 36, 15356 (Dec. 9, 1997).
43. X. Wang, C. Yang, J. Chai, Y. Shi, D. Xue, Mechanisms of AIF-mediated apoptotic DNA degradation in Caenorhabditis elegans. Science 298, 1587 (Nov. 22, 2002).
44. C. Cande et al., AIF and cyclophilin A cooperate in apoptosis-associated chromatinolysis. Oncogene 23, 1514 (Feb. 26, 2004).
45. C. Artus et al., AIF promotes chromatinolysis and caspase-independent programmed necrosis by interacting with histone H2AX. EMBO J 29, 1585 (May 5, 2010).
46. A. Athanasiadis et al., Crystal structure of PvuII endonuclease reveals extensive structural homologies to EcoRV. Nat Struct Biol 1, 469 (July, 1994).
47. J. D. Hudson et al., A proinflammatory cytokine inhibits p53 tumor suppressor activity. J Exp Med 190, 1375 (Nov. 15, 1999).
48. R. Kleemann et al., Intracellular action of the cytokine MIF to modulate AP-1 activity and the cell cycle through Jab1. Nature 408, 211 (Nov. 9, 2000).
49. A. R. Inacio, K. Ruscher, L. Leng, R. Bucala, T. Deierborg, Macrophage migration inhibitory factor promotes cell death and aggravates neurologic deficits after experimental stroke. J Cereb Blood Flow Metab 31, 1093 (April, 2011).
50. R. A. Irvine et al., Generation and characterization of endonuclease G null mice. Mol Cell Biol 25, 294 (January, 2005).
51. ChTPseq data (GSE65110) www.ncbi.nlm.nih.gov/geo/query/acc.cgi?token=atktuskchzmbvgz&acc=GSE65110.
52. ChTPseq.bam files:
   drive.google.com/folderview?id=0B5rxNsjjvI-9fjJfS1FXbU9vMDFUR1ZBSC0yYkswZ3NjYzZMVVBiYUoySG5YOVRzQTZk W8&usp=sharing.

1-6. (canceled)

7. A method of screening candidate compounds for macrophage migration inhibitory factor (MIF) inhibitors comprising:
(a) immobilizing a single-stranded amine modified MIF target DNA on a surface;
(b) incubating the MIF target DNA of (a) with an MIF protein and a candidate MIF inhibitor compound;
(c) hybridizing the MIF target DNA with a complementary biotinylated DNA;
(d) measuring a nuclease activity of the MIF protein; and
(e) identifying the candidate MIF inhibitor as a MIF inhibitor when the candidate MIF inhibitor blocks the MIF protein nuclease activity,
thereby screening for MIF inhibitors.

8. The method of claim 7, wherein the candidate MIF inhibitor compound is a macrocyclic compound comprising a FKBP-binding domain and a tetra-peptidyl effector domain.

9. The method of claim 7, wherein the MIF inhibitor disrupts MIF/AIF interaction, inhibits MIF-induced chromatinolysis, inhibits MIF-induced cell death, and/or prevents MIF translocation to the nucleus of a cell.

10. The method of claim 7, wherein measuring nuclease activity of the recombinant MIF comprises:
(i) incubating the MIF target DNA, the MIF protein, the candidate MIF inhibitor and the complementary biotinylated DNA with a streptavidin enzyme conjugate;
(ii) incubating the MIF target DNA, the MIF protein, the candidate MIF inhibitor, the complementary biotinylated DNA and the streptavidin enzyme conjugate with a streptavidin substrate, wherein the substrate is acted upon by the streptavidin enzyme conjugate; and
(iii) measuring an absorbance, wherein the absorbance determines the MIF nuclease activity.

11. The method of claim 10, wherein an absence of a change in absorbance indicates that the MIF target DNA is not cleaved, that there is no MIF nuclease activity and/or that the candidate MIF inhibitor is a MIF inhibitor.

12. The method of claim 10, wherein a change in the absorbance indicates that the MIF target DNA is cleaved, that there is MIF nuclease activity and/or that the candidate MIF inhibitor is not a MIF inhibitor.

13. The method of claim 10, wherein the candidate MIF inhibitor compound identified as a MIF inhibitor is used for treating a disease characterized by increased poly [ADP-ribose]polymerase 1 (PARP-1).

14. The method of claim 13, wherein the disease is an inflammatory disease.

15. The method of claim 14, wherein the inflammatory disease is selected from the group consisting of Alzheimer's, ankylosing spondylitis, arthritis, osteoarthritis, rheumatoid arthritis, psoriatic arthritis, asthma atherosclerosis, Crohn's disease, colitis, dermatitis diverticulitis, fibromyalgia, hepatitis, irritable bowel syndrome, systemic lupus erythematous, nephritis, ulcerative colitis and Parkinson's disease.

16. The method of claim 3, wherein the disease is Parkinson's disease.

17. A kit for screening candidate compounds for macrophage migration inhibitory factor (MIF) inhibitors comprising:
(i) a single-stranded amine modified MIF target DNA;
(ii) a recombinant MIF protein;
(iii) a biotinylated DNA complementary to the MIF target DNA of (i); and
(iv) instructions for screening candidate compounds for MIF inhibitors.

18. The kit of claim 17, wherein the single-stranded amine modified MIF target DNA is immobilized on a surface.

19. The kit of claim 17, wherein the instructions comprise instructions for measuring a nuclease activity of the recombinant MIF.

20. The kit of claim 17, further comprising a streptavidin enzyme conjugate and a streptavidin substrate.


SEQUENCE LISTING (170 sequences)

SEQ ID NO: 1 - DNA, 42 bp, Artificial Sequence
  Synthetic
cagaaggttaattaaaaggtatattgctgttgacagtgagcg

SEQ ID NO: 2 - DNA, 35 bp, Artificial Sequence
  Synthetic
ctaaagtagccccttgctagccgaggcagtaggca

SEQ ID NO: 3 - DNA, 100 bp, Artificial Sequence
  Synthetic
acctaaatgctagagctcgctgatcagcctcgactctcagcctcccaagtagctgggattacaggtaaacttggtctgacagttaccaatgcttaatgag

SEQ ID NO: 4 - DNA, 100 bp, Artificial Sequence
  Synthetic
ctcattaagcattggtaactgtcagaccaagtttacctgtaatcccagctacttgggaggctgagagtcgaggctgatcagcgagctctagcatttaggt

SEQ ID NO: 5 - DNA, 30 bp, Artificial Sequence
  Synthetic
ctcagcctcccaagtagctgggattacagg

SEQ ID NO: 6 - DNA, 30 bp, Artificial Sequence
  Synthetic
cctgtaatcccaagtagctgggattacagg

SEQ ID NO: 7 - DNA, 30 bp, Artificial Sequence
  Synthetic
aaaaaaactcagcctcccaagtagctggga

SEQ ID NO: 8 - DNA, 30 bp, Artificial Sequence
  Synthetic
tcccaagtagctgggattacaggaaaaaaa

SEQ ID NO: 9 - DNA, 30 bp, Artificial Sequence
  Synthetic
aaaaaaaaaaaaaaaaaaaaaaaaaaaaaa

SEQ ID NO: 10 - DNA, 30 bp, Artificial Sequence; identical to SEQ ID NO: 5
  Synthetic

SEQ ID NO: 11 - DNA, 40 bp, Artificial Sequence
  Synthetic
ctcagcctcccaagtagctgggattacaggtaaacttggt

SEQ ID NO: 12 - DNA, 30 bp, Artificial Sequence
  Synthetic
aaaaaaaaaacaagtagctgggattacagg

SEQ ID NO: 13 - DNA, 30 bp, Artificial Sequence
  Synthetic
acctaaatgctagagctcgctgatcagcct

SEQ ID NO: 14 - DNA, 97 bp, Artificial Sequence
  Synthetic
tgctgttgacagtgagcgctcatcgtaaacaccaacgtgctagtgaagccacagatgtagcacgttggtgtttacgatgaatgcctactgcctcgga

SEQ ID NO: 15 - DNA, 96 bp, Artificial Sequence
  Synthetic
tgctgttgacagtgagcgacgcgcagaaccgctcctacagtagtgaagccacagatgtactgtaggagcggttctgcgcgctgcctactgcctcgg

SEQ ID NO: 16 - DNA, 97 bp, Artificial Sequence
  Synthetic
tgctgttgacagtgagcgaagggtctacatcaactattactagtgaagccacagatgtagtaatagttgatgtagaccctgtgcctactgcctcgga

SEQ ID NO: 17 - DNA, 96 bp, Artificial Sequence
  Synthetic
gctgttgacagtgagcgctcatcgtgaacaccaatgttctagtgaagccacagatgtagaacattggtgttcacgatgaatgcctactgcctcgga

SEQ ID NO: 18 - DNA, 97 bp, Artificial Sequence
  Synthetic
tgctgttgacagtgagcgagcagtgcacgtggtcccggactagtgaagccacagatgtagtccgggaccacgtgcactgcgtgcctactgcctcgga

SEQ ID NO: 19 - DNA, 97 bp, Artificial Sequence
  Synthetic
tgctgttgacagtgagcgacgggtctacatcaactattactagtgaagccacagatgtagtaatagttgatgtagacccggtgcctactgcctcgga

SEQ ID NO: 20 - DNA, 97 bp, Artificial Sequence
  Synthetic
tgctgttgacagtgagcgcggaaccggcttccagctacagtagtgaagccacagatgtactgtagctggaagccggttccttgcctactgcctcgga

SEQ ID NO: 21 - DNA, 35 bp, Artificial Sequence
  Synthetic
cgggatccgccaccatgcctatgttcatcgtgaac

SEQ ID NO: 22 - DNA, 28 bp, Artificial Sequence
  Synthetic
cggaattctcaagcgaaggtggaaccgt

SEQ ID NO: 23 - DNA, 49 bp, Artificial Sequence
  Synthetic
caccatgcctatgtttattgtcaatacgaacgtaccccgcgcctccgtg

SEQ ID NO: 24 - DNA, 49 bp, Artificial Sequence
  Synthetic
cacggaggcgcggggtacgttcgtattgacaataaacataggcatggtg

SEQ ID NO: 25 - DNA, 52 bp, Artificial Sequence
  Synthetic
gcacatcagcccggaccgcgtgtatattaattactatgacatgaacgctgcc

SEQ ID NO: 26 - DNA, 52 bp, Artificial Sequence
  Synthetic
ggcagcgttcatgtcatagtaattaatatacacgcggtccgggctgatgtgc

SEQ ID NO: 27 - DNA, 37 bp, Artificial Sequence
  Synthetic
gggatccccggaattcgggatgttcatcgtaaacacc

SEQ ID NO: 28 - DNA, 37 bp, Artificial Sequence
  Synthetic
ggtgtttacgatgaacatcccgaattccggggatccc

SEQ ID NO: 29 - DNA, 20 bp, Artificial Sequence
  Synthetic
cctccgtggcggacgggttc

SEQ ID NO: 30 - DNA, 20 bp, Artificial Sequence
  Synthetic
gaacccgtccgccacggagg

SEQ ID NO: 31 - DNA, 30 bp, Artificial Sequence
  Synthetic
cgcgcctccgtgccggccgggttcctctcc

SEQ ID NO: 32 - DNA, 30 bp, Artificial Sequence
  Synthetic
ggagaggaacccggccggcacggaggcgcg

SEQ ID NO: 33 - DNA, 20 bp, Artificial Sequence
  Synthetic
ccgtgccgcaagggttcctc

SEQ ID NO: 34 - DNA, 20 bp, Artificial Sequence
  Synthetic
gaggaacccttgcggcacgg

SEQ ID NO: 35 - DNA, 30 bp, Artificial Sequence
  Synthetic
gggttcctctccgcgctcacccagcagctg

SEQ ID NO: 36 - DNA, 30 bp, Artificial Sequence
  Synthetic
cagctgctgggtgagcgcggagaggaaccc

SEQ ID NO: 37 - DNA, 30 bp, Artificial Sequence
  Synthetic
gggttcctctcccagctcacccagcagctg

SEQ ID NO: 38 - DNA, 30 bp, Artificial Sequence
  Synthetic
cagctgctgggtgagctgggagaggaaccc

SEQ ID NO: 39 - DNA, 30 bp, Artificial Sequence
  Synthetic
gggttcctctccgacctcacccagcagctg

SEQ ID NO: 40 - DNA, 30 bp, Artificial Sequence
  Synthetic
cagctgctgggtgaggtcggagaggaaccc

SEQ ID NO: 41 - DNA, 20 bp, Artificial Sequence
  Synthetic
gtgcacgtggtcgcggacca

SEQ ID NO: 42 - DNA, 20 bp, Artificial Sequence
  Synthetic
catgagctggtccgcgacca

SEQ ID NO: 43 - DNA, 31 bp, Artificial Sequence
  Synthetic
gcacgtggtcccggcccagctcatggccttc

SEQ ID NO: 44 - DNA, 31 bp, Artificial Sequence
  Synthetic
gaaggccatgagctgggccgggaccacgtgc

SEQ ID NO: 45 - DNA, 20 bp, Artificial Sequence
  Synthetic
gtggtcccgcaacagctcat

SEQ ID NO: 46 - DNA, 20 bp, Artificial Sequence
  Synthetic
ccatgagctgttgcgggacc

SEQ ID NO: 47 - DNA, 33 bp, Artificial Sequence
  Synthetic
cttcggcggctccagcgcgccgtgcgcgctctg

SEQ ID NO: 48 - DNA, 33 bp, Artificial Sequence
  Synthetic
cagagcgcgcacggcgcgctggagccgccgaag

SEQ ID NO: 49 - DNA, 20 bp, Artificial Sequence
  Synthetic
ctccagccagccgtgcgcgc

SEQ ID NO: 50 - DNA, 20 bp, Artificial Sequence
  Synthetic
gcgcgcacggctggctggag

SEQ ID NO: 51 - DNA, 30 bp, Artificial Sequence
  Synthetic
ggcctgctggccgcgcgcctgcgcatcagc

SEQ ID NO: 52 - DNA, 30 bp, Artificial Sequence
  Synthetic
gctgatgcgcaggcgcgcggccagcaggcc

SEQ ID NO: 53 - DNA, 24 bp, Artificial Sequence
  Synthetic
gctggccgagcaactgcgcatcag

SEQ ID NO: 54 - DNA, 24 bp, Artificial Sequence
  Synthetic
ctgatgcgcagttgctcggccagc

SEQ ID NO: 55 - DNA, 20 bp, Artificial Sequence
  Synthetic
ccgagcgcctgcaaatcagc

SEQ ID NO: 56 - DNA, 20 bp, Artificial Sequence
  Synthetic
gctgatgcgcagttgctcgg

SEQ ID NO: 57 - DNA, 20 bp, Artificial Sequence
  Synthetic
gcgcatcagcgcggacaggg

SEQ ID NO: 58 - DNA, 20 bp, Artificial Sequence
  Synthetic
ccctgtccgcgctgatgcgc

SEQ ID NO: 59 - DNA, 33 bp, Artificial Sequence
  Synthetic
ctgcgcatcagcccggccagggtctacatcaac

SEQ ID NO: 60 - DNA, 33 bp, Artificial Sequence
  Synthetic
gttgatgtagaccctggccgggctgatgcgcag

SEQ ID NO: 61 - DNA, 20 bp, Artificial Sequence
  Synthetic
cagcccgcaaagggtctaca

SEQ ID NO: 62 - DNA, 20 bp, Artificial Sequence
  Synthetic
tgtagaccctttgcgggctg

SEQ ID NO: 63 - DNA, 28 bp, Artificial Sequence
  Synthetic
catcaactattacgccatgaacgcggcc

SEQ ID NO: 64 - DNA, 28 bp, Artificial Sequence
  Synthetic
ggccgcgttcatggcgtaatagttgatg

SEQ ID NO: 65 - DNA, 49 bp, Artificial Sequence
  Synthetic
cggcggctccagcgagccggccgcgctcgccagcctgcacagcatcggc

SEQ ID NO: 66 - DNA, 49 bp, Artificial Sequence
  Synthetic
gccgatgctgtgcaggctggcgagcgcggccggctcgctggagccgccg

SEQ ID NO: 67 - DNA, 33 bp, Artificial Sequence
  Synthetic
gaggggtttctgtcggacctcacccagcagctg

SEQ ID NO: 68 - DNA, 33 bp, Artificial Sequence
  Synthetic
cagctgctgggtgaggtccgacagaaacccctc

SEQ ID NO: 69 - DNA, 33 bp, Artificial Sequence
  Synthetic
gaggggtttctgtcgcagctcacccagcagctg

SEQ ID NO: 70 - DNA, 33 bp, Artificial Sequence
  Synthetic
cagctgctgggtgagctgcgacagaaacccctc

SEQ ID NO: 71 - DNA, 31 bp, Artificial Sequence
  Synthetic
cggatccgccaccatgcctatgttcatcgtg

SEQ ID NO: 72 - DNA, 52 bp, Artificial Sequence
  Synthetic
cggaattctcacttgtcgtcgtcgtccttgtagtcagcgaaggtggaaccgt

SEQ ID NO: 73 - DNA, 31 bp, Artificial Sequence
  Synthetic
cggaattcatgcgggcgctgcgggccggcct

SEQ ID NO: 74 - DNA, 31 bp, Artificial Sequence
  Synthetic
ccgctcgagtcacttactgcccgccgtgatg

SEQ ID NO: 75 - DNA, 29 bp, Artificial Sequence
  Synthetic
cggaattcatggtcaaccccaccgtgttc

SEQ ID NO: 76 - DNA, 32 bp, Artificial Sequence
  Synthetic
ccgctcgagttattcgagttgtccacagtcag

SEQ ID NO: 77 - DNA, 23 bp, Artificial Sequence
  Synthetic
ccgccgccgccaaccaccgctgg

SEQ ID NO: 78 - DNA, 23 bp, Artificial Sequence
  Synthetic
gggctgggtgcggtcgtcgaggg

SEQ ID NO: 79 - DNA, 30 bp, Artificial Sequence
  Synthetic
ctcagcctccaaaaaaaaaaggattacagg

SEQ ID NO: 80 - DNA, 30 bp, Artificial Sequence
  Synthetic
ctcagcctcccaagtagctgaaaaaaaaaa

SEQ ID NO: 81 - DNA, 30 bp, Artificial Sequence
  Synthetic
ctcagccaaaaaaatagctgggattacagg

SEQ ID NO: 82 - DNA, 30 bp, Artificial Sequence
  Synthetic
ctcagcctcccaaaaaaaagggattacagg

SEQ ID NO: 83 - DNA, 30 bp, Artificial Sequence
  Synthetic
ctcaaaaaaacaagtagctgggattacagg

SEQ ID NO: 84 - DNA, 30 bp, Artificial Sequence
  Synthetic
aaaagcctcccaagtagctgggattacagg

SEQ ID NO: 85 - DNA, 30 bp, Artificial Sequence
  Synthetic
ctcaaaatcccaaaaaaaagggattacagg

SEQ ID NO: 86 - DNA, 30 bp, Artificial Sequence
  Synthetic
caagtagctgctcagcctccggattacagg

SEQ ID NO: 87 - DNA, 30 bp, Artificial Sequence
  Synthetic
ctcagcctccggattacaggcaagtagctg

SEQ ID NO: 88 - DNA, 30 bp, Artificial Sequence
  Synthetic
ctcagcctcccaagtaaaagggattacagg

SEQ ID NO: 89 - DNA, 30 bp, Artificial Sequence
  Synthetic
ctcagcctcccaagtaaatgggattacagg

SEQ ID NO: 90 - DNA, 30 bp, Artificial Sequence
  Synthetic
ctcagcctcccaagtaaacgggattacagg

SEQ ID NO: 91 - DNA, 30 bp, Artificial Sequence
  Synthetic
ctcagcctcccaagtagcagggattacagg

SEQ ID NO: 92 - DNA, 30 bp, Artificial Sequence
  Synthetic
ctcagcctcccttgtagctgggattacagg

SEQ ID NO: 93 - DNA, 30 bp, Artificial Sequence
  Synthetic
ctcaaaatcccaagtagctgggattacagg

SEQ ID NO: 94 - DNA, 30 bp, Artificial Sequence
  Synthetic
ctcaatttcccaagtagctgggattacagg

SEQ ID NO: 95 - DNA, 30 bp, Artificial Sequence
  Synthetic
aaaaatctcagcctcccaagtagctgggat

SEQ ID NO: 96 - DNA, 29 bp, Artificial Sequence
  Synthetic
tgggattacaggcgtgagccaccacgccc

SEQ ID NO: 97 - protein, 37 aa, Homo sapiens
MPMFIVNTNVPRASVPDGFLSELTQQLAQATGKPPQY

SEQ ID NO: 98 - protein, 53 aa, Unknown
  Description of Unknown ExoIII sequence
MKFVSFNINGLRARPHQLEAIVEKHQPDVIGLQETKVHDDMFPLEEVAKLGYN

SEQ ID NO: 99 - protein, 53 aa, Unknown
  Description of Unknown APE1 sequence
LKIATFNVNSIRSRLHIVIPWLKENKPDILCMQETKVENRKFPEADFHRIGYH

SEQ ID NO: 100 - protein, 19 aa, Unknown
  Description of Unknown StsI sequence
NKPDGLLWDDDCAIILDSK

SEQ ID NO: 101 - protein, 26 aa, Unknown
  Description of Unknown EcoRI sequence
IKPDGGIVEVKDDYGEWRVVLVAEAK

SEQ ID NO: 102 - protein, 23 aa, Unknown
  Description of Unknown FokI sequence
RKPDGAIYTVGSPIDYGVIVDTK

SEQ ID NO: 103 - protein, 26 aa, Unknown
  Description of Unknown RsrI sequence
IRPDGGITEVKDRHGNWRVILVGESK

SEQ ID NO: 104 - protein, 22 aa, Unknown
  Description of Unknown EcoRV sequence
HYPDFTLYKPSEPNKKIAIDIK

SEQ ID NO: 105 - protein, 30 aa, Artificial Sequence
  Description of Artificial Sequence Synthetic polypeptide
  MOD_RES (3)..(3): G, V, I, or F
  MOD_RES (4)..(4): F, I, L, G, A, or T
  MOD_RES (5)..(5): L, G, C, or I
  MOD_RES (6)..(6): L, M, Y, T, or not present
  MOD_RES (7)..(7): T, E, K, or not present
  MOD_RES (8)..(8): V, P, or not present
  MISC_FEATURE (9)..(11): May or may not be present
  MOD_RES (12)..(12): S, Q, W, V, I, or K
  MOD_RES (14)..(14): L, T, D, V, Y, R, or P
  MOD_RES (15)..(15): T, K, D, H, N, or not present
  MOD_RES (16)..(16): Q, V, C, D, K, or not present
  MOD_RES (17)..(17): Q, H, E, D, or not present
  MOD_RES (18)..(18): L, D, N, Y, or not present
  MOD_RES (19)..(19): D, R, G, or not present
  MOD_RES (20)..(20): M, K, E, N, or not present
  MOD_RES (21)..(21): F, W, or not present
  MOD_RES (22)..(22): A, P, R, or not present
  MOD_RES (23)..(23): Q, L, E, V, or not present
  MOD_RES (24)..(24): A, E, V, I, or K
  MOD_RES (25)..(25): T, E, D, I, or L
  MOD_RES (26)..(26): V, F, I, A, or not present
  MOD_RES (27)..(27): A, H, L, G, I, or not present
  MOD_RES (28)..(28): D, E, or not present
  MOD_RES (29)..(29): G, S, A, T, I, or not present
PDXXXXXXGSPXEXXXXXXXXXXXXXXXXK

SEQ ID NO: 106 - protein, 51 aa, Homo sapiens
IAVHVVPDQLMAFGGSSEPCALCSLHSIGKIGGAQNRSYSKLLCGLLAERL

SEQ ID NO: 107 - protein, 25 aa, Artificial Sequence
  Description of Artificial Sequence Synthetic peptide
CALCSLHSIGKIGGAQNRSYSKLLC

SEQ ID NO: 108 - protein, 24 aa, Artificial Sequence
  Description of Artificial Sequence Synthetic peptide
PDQLMAFGGSSEPCALCSLHSIGK

SEQ ID NO: 109 - protein, 27 aa, Homo sapiens
RISPDRVYINYYDMNAANVGWNNSTFA

SEQ ID NO: 110 - protein, 10 aa, Artificial Sequence
  Description of Artificial Sequence Synthetic peptide
PDRVYINYYD

SEQ ID NO: 111 - DNA, 30 bp, Artificial Sequence
  Description of Artificial Sequence Synthetic oligonucleotide
ctyrsccyycyrmrywkmtsggmyyayagr

SEQ ID NO: 112 - DNA, 28 bp, Artificial Sequence
  Description of Artificial Sequence Synthetic oligonucleotide
samsmswysmwcmyrgckmmgcmcssyr

SEQ ID NO: 113 - DNA, 29 bp, Artificial Sequence
  Description of Artificial Sequence Synthetic oligonucleotide
trggayyayagryryswrccacyrykyyy

SEQ ID NO: 114 - DNA, 15 bp, Artificial Sequence
  Description of Artificial Sequence Synthetic oligonucleotide
mmrwrrrrwwmwrrr

SEQ ID NO: 115 - DNA, 30 bp, Unknown; identical to SEQ ID NO: 5
  Description of Unknown MIF DNA binding sequence

SEQ ID NO: 116 - DNA, 25 bp, Unknown
  Description of Unknown MIF DNA binding sequence
ctcagcctcccaagtagctgggatt

SEQ ID NO: 117 - DNA, 27 bp, Unknown
  Description of Unknown MIF DNA binding sequence
aaaaaaaaaagtagctgggattacagg

SEQ ID NO: 118 - protein, 19 aa, Homo sapiens
VPDGFLSELTQQLAQATGK

SEQ ID NO: 119 - protein, 25 aa, Homo sapiens
VPDQLMAFGGSSEPCALCSLHSIGK

SEQ ID NO: 120 - protein, 11 aa, Homo sapiens
SPDRVYINYYD

SEQ ID NO: 121 - protein, 19 aa, Mus sp.
VPEGFLSELTQQLAQATGK

SEQ ID NO: 122 - protein, 25 aa, Mus sp.
VPDQLMTFSGTNDPCALCSLHSIGK

SEQ ID NO: 123 - protein, 11 aa, Mus sp.
SPDRVYINYYD

SEQ ID NO: 124 - protein, 19 aa, Rattus sp.
VPEGFLSELTQQLAQATGK

SEQ ID NO: 125 - protein, 25 aa, Rattus sp.
VPDQLMTFSGTSDPCALCSLHSIGK

SEQ ID NO: 126 - protein, 11 aa, Rattus sp.
SPDRVYINYYD

SEQ ID NO: 127 - protein, 19 aa, Unknown
  Description of Unknown monkey MIF sequence
VPDGFLSELTQQLAQATGK

SEQ ID NO: 128 - protein, 25 aa, Unknown
  Description of Unknown monkey MIF sequence
VPDQLMAFGGSSEPCALCSLHSIGK

SEQ ID NO: 129 - protein, 11 aa, Unknown
  Description of Unknown monkey MIF sequence
SPDRVYINYYD

SEQ ID NO: 130 - protein, 19 aa, Sus sp.
VPDGFLSELTQQLVQAMGK

SEQ ID NO: 131 - protein, 25 aa, Sus sp.
VPDQLMAFGGSSEPCALCSLHSIGK

SEQ ID NO: 132 - protein, 11 aa, Sus sp.
SPDRIYINYYD

SEQ ID NO: 133 - protein, 19 aa, Bos sp.
VPDGLLSELTQQLAQATGK

SEQ ID NO: 134 - protein, 25 aa, Bos sp.
VPDQLMTFGGSSEPCALCSLHSIGK

SEQ ID NO: 135 - protein, 11 aa, Bos sp.
SPDRIYINFCD

SEQ ID NO: 136 - protein, 19 aa, Ovis sp.
VPDGLLSELTQQLAQATGK

SEQ ID NO: 137 - protein, 25 aa, Ovis sp.
VPDQLMTFGGSSEPCALCSLHSIGK

SEQ ID NO: 138 - protein, 11 aa, Ovis sp.
SPDRIYINFCD

SEQ ID NO: 139 - protein, 19 aa, Oryctolagus cuniculus
VPDGLLSELTQQLAQATGK

SEQ ID NO: 140 - protein, 25 aa, Oryctolagus cuniculus
VPDQLMAFGGSSEPCALCSLHSIGK

SEQ ID NO: 141 - protein, 11 aa, Oryctolagus cuniculus
SPDRIYINYFD

SEQ ID NO: 142 - protein, 19 aa, Sorex sp.
VPDGLLAELTQQLAQATGK

SEQ ID NO: 143 - protein, 25 aa, Sorex sp.
VPDQLMAFGGSSEPCALCSLHSIGK

SEQ ID NO: 144 - protein, 11 aa, Sorex sp.
SPDRIYINYYD

SEQ ID NO: 145 - protein, 18 aa, Artificial Sequence
  Description of Artificial Sequence Synthetic peptide
  MOD_RES (2)..(2): D or E
  MOD_RES (4)..(4): F or L
  MOD_RES (6)..(6): S or A
  MOD_RES (13)..(13): A or V
  MOD_RES (16)..(16): T or M
PXGXLXELTQQLXQAXGK

SEQ ID NO: 146 - protein, 24 aa, Artificial Sequence
  Description of Artificial Sequence Synthetic peptide
  MOD_RES (4)..(4): L or I
  MOD_RES (6)..(6): A or T
  MOD_RES (8)..(8): G or S
  MOD_RES (10)..(10): S or T
  MOD_RES (11)..(11): S or N
PDQXMXFXGXXEPCALCSLHSIGK

SEQ ID NO: 147 - protein, 10 aa, Artificial Sequence
  Description of Artificial Sequence Synthetic peptide
  MOD_RES (4)..(4): V or I
  MOD_RES (8)..(8): Y or F
  MOD_RES (9)..(9): Y, C, or F
PDRXYINXXD

SEQ ID NO: 148 - protein, 33 aa, Homo sapiens
PCALCSLHSIGKIGGAQNRSYSKLLCGLLAERL

SEQ ID NO: 149 - protein, 33 aa, Mus sp.
PCALCSLHSIGKIGGAQNRNYSKLLCGLLSDRL

SEQ ID NO: 150 - protein, 33 aa, Rattus sp.; identical to SEQ ID NO: 149

SEQ ID NO: 151 - protein, 33 aa, Unknown; identical to SEQ ID NO: 148
  Description of Unknown monkey MIF sequence

SEQ ID NO: 152 - protein, 33 aa, Sus sp.; identical to SEQ ID NO: 148

SEQ ID NO: 153 - protein, 33 aa, Bos sp.
PCALCSLHSIGKIGGAQNRSYSKLLCGLLTERL

SEQ ID NO: 154 - protein, 33 aa, Ovis sp.; identical to SEQ ID NO: 153

SEQ ID NO: 155 - protein, 33 aa, Oryctolagus cuniculus
PCALCSLHSIGKIGGAQNRTYSKLLCGLLSERL

SEQ ID NO: 156 - protein, 33 aa, Sorex sp.
PCALCSLHSIGKIGGAQNRTYSKLLCNLLTERL

SEQ ID NO: 157 - protein, 25 aa, Artificial Sequence
  Description of Artificial Sequence Synthetic peptide
  MOD_RES (19)..(19): S, N, or T
CALCSLHSIGKIGGAQNRXYSKLLC

SEQ ID NO: 158 - DNA, 28 bp, Unknown
  Description of Unknown MIF DNA binding sequence
  modified_base (12)..(12): a, c, t, g, unknown or other
  modified_base (16)..(16): a, c, t, g, unknown or other
  modified_base (24)..(24): a, c, t, g, unknown or other
caagccatcctnccanctcagccnccca

SEQ ID NO: 159 - DNA, 29 bp, Unknown
  Description of Unknown MIF DNA binding sequence
tgggattacaggcgtgagccaccacgccc

SEQ ID NO: 160 - DNA, 30 bp, Unknown; identical to SEQ ID NO: 6
  Description of Unknown MIF DNA binding sequence

SEQ ID NO: 161 - DNA, 30 bp, Unknown; identical to SEQ ID NO: 7
  Description of Unknown MIF DNA binding sequence

SEQ ID NO: 162 - DNA, 30 bp, Unknown
  Description of Unknown MIF DNA binding sequence
tccgaagtagctcggattacaggaaaaaaa

SEQ ID NO: 163 - DNA, 22 bp, Unknown
  Description of Unknown MIF DNA binding sequence
ctcagcctcccaagtacttggt

SEQ ID NO: 164 - DNA, 30 bp, Unknown
  Description of Unknown MIF DNA binding sequence
acctaaatgctagctcgtgggattacacct

SEQ ID NO: 165 - DNA, 10 bp, Unknown
  Description of Unknown MIF DNA binding sequence
aaaaaaaaaa

SEQ ID NO: 166 - DNA, 10 bp, Unknown
  Description of Unknown MIF DNA binding sequence
agtctttttt

SEQ ID NO: 167 - DNA, 14 bp, Unknown
  Description of Unknown MIF DNA binding sequence
aaaaaaaaaagact

SEQ ID NO: 168 - DNA, 14 bp, Unknown
  Description of Unknown MIF DNA binding sequence
agtctttttttttt

SEQ ID NO: 169 - DNA, 17 bp, Unknown
  Description of Unknown MIF DNA binding sequence
ctcaggctgggattagg

SEQ ID NO: 170 - DNA, 12 bp, Unknown
  Description of Unknown MIF DNA binding sequence
tcccagcctgag